(19) United States
(12) Patent Application Publication
(10) Pub. No.: US 2024/0400672 A1
(43) Pub. Date: Dec. 5, 2024

(54) NOVEL MONOCLONAL ANTIBODIES DIRECTED AGAINST L-THYROXINE AND DIAGNOSTIC USES THEREOF

(71) Applicant: Roche Diagnostics Operations, Inc., Indianapolis, IN (US)

(72) Inventors: Michael Gerg, Muenchen (DE); Klaus Hirzel, Baierbrunn (DE); Caroline Dorothea Hojer, München (DE); Hans-Peter Josel, Weilheim (DE); Ute Jucknischke, Iffeldorf (DE); Holger Lehmann, Marburg (DE); Anastasia Markina, Munich (DE); Giuseppe Prencipe, Königsdorf (DE); Leopold von Proff, Hohenpeissenberg (DE); Michael Schraeml, Penzberg (DE); Steinar Halldórsson, Basel (CH)

(21) Appl. No.: 18/662,694

(22) Filed: May 13, 2024

Related U.S. Application Data
(63) Continuation of application No. PCT/EP2022/080897, filed on Nov. 7, 2022.

(30) Foreign Application Priority Data
Nov. 11, 2021 (EP) 21207858.8

(51) Int. Cl. C07K 16/26; G01N 33/78

(52) U.S. Cl. CPC C07K 16/26; G01N 33/78; C07K 2317/33; C07K 2317/55; C07K 2317/92; G01N 2470/10

(57) ABSTRACT

The present invention provides a novel monoclonal antibody specifically binding to L-Thyroxine (T4) and compositions and kits comprising such antibodies. Furthermore, provided are polynucleotides encoding such monoclonal antibodies, host cells expressing said antibodies, methods of producing such antibodies and diagnostic methods using such monoclonal antibodies. The monoclonal antibody of the invention comprises a heavy chain variable domain (VH) comprising V or A in position 33; Y in position 50; W in position 52; I in position 98, G, A or V in position 99; Y in position 100; and I in position 100b; and a light chain variable domain (VL) comprising amino acids H or Y in position 28; N or K in position 29; W in position 32; G or A in position 91; Y, W or F in position 92; S or T in position 93; Y or F in position 95b; N, S, T or Q in position 95c; and H in position 96, wherein the positions of the amino acids in the VH and the VL are indicated according to the Kabat numbering scheme, respectively.

### CROSS-REFERENCE TO RELATED APPLICATIONS

This application is a continuation of International PCT Application No. PCT/EP2022/080897 filed on Nov. 7, 2022, which claims priority to European Patent Application No. 21207858.8 filed on Nov. 11, 2021, the contents of each application are incorporated herein by reference in their entireties.

### SEQUENCE LISTING

This application incorporates by reference the material in the ST.26 XML file titled P37213-US_Sequence-Listing_ROCHE-121, which was created on May 8, 2024 and is 89 KB.

The present invention provides novel monoclonal antibodies directed to L-thyroxine (T4) and compositions and kits comprising such antibodies. Furthermore, provided are polynucleotides encoding such monoclonal antibodies, host cells expressing said antibodies, methods of producing such antibodies and diagnostic methods using such monoclonal antibodies.

L-thyroxine hormone (herein subsequently referred to as L-T4 or T4; CAS No.: 51-48-9) is the main thyroid hormone secreted into the bloodstream by the thyroid gland. Together with triiodothyronine (T3) it plays a vital role in regulating the body's metabolic rate, influences the cardiovascular system, growth and bone metabolism, and is important for normal development of gonadal functions and nervous system (Kronenberg H M et al. Williams Textbook of Endocrinology. Saunders Elsevier, Philadelphia, 12th edition, 2011, chapter 10, p. 301-311). T4 circulates in the bloodstream as an equilibrium mixture of free and serum bound hormone. Free T4 (fT4) is the unbound and biologically active form, which represents only 0.03% of the total T4. The remaining T4 is inactive and bound to serum proteins such as thyroxine binding globulin (TBG) (75%), pre-albumin (15%), and albumin (10%) (Robbins J, Rall J E. Recent Prog Horm Res 1957; 13:161-208; Oppenheimer J H. N Engl J Med 1968; 278(21):1153-1162; DeGroot L J, Larsen P R, Hennemann G. Wiley and Sons, New York, 1984:62-65; Ekins R P. Endocr Rev 1990; 11(1):5-46). The determination of free T4 has the advantage of being independent of changes in the concentrations and binding properties of these binding proteins; additional determination of a binding parameter (T uptake, TBG) is therefore unnecessary. Thus free T4 is a useful tool in clinical routine diagnostics for the assessment of the thyroid status. It should be measured together with TSH if thyroid disorders are suspected and is also suitable for monitoring thyrosuppressive therapy (Kronenberg H M, Melmed S, Polonsky K S, et al. Williams Textbook of Endocrinology. Saunders Elsevier, Philadelphia, 12th edition, 2011, chapter 10, p. 301-311; Wu A H B. Tietz Clinical Guide To Laboratory Tests. Saunders Elsevier, Philadelphia, 4th edition, 2006, section II, p. 1076-1077; Brent G A. Thyroid Function Testing. Springer, Berlin, 1st edition, 2010, chapter 5, p. 86-88).

A variety of methods is available for estimating the free thyroid hormone levels. The direct measurement of fT4 and fT3 via equilibrium dialysis or ultrafiltration is mainly used as a reference method for standardizing, the immunological procedures generally used for routine diagnostic purposes (Wu A H B. Tietz Clinical Guide To Laboratory Tests. Saunders Elsevier, Philadelphia, 4th edition, 2006, section II, p. 1076-1077; Brent G A. Thyroid Function Testing. Springer, Berlin, 1st edition, 2010, chapter 5, p. 86-88).

A number of commercially available fully automated fT4 and total T4 immunoassays use polyclonal antibodies raised in e.g. sheep or rabbits. However, several disadvantages arise from the use of polyclonal antibodies for a routine in vitro diagnostic product: 1) Due to the origin from animal serum, the majority of an antibody preparation initially exists of nonspecific IgGs, so usually the target-specific antibody has to be purified, e.g. using affinity chromatography, which during the elution step exposes the antibody to unfavourable conditions, for example low pH or otherwise denaturing conditions, which can lead to enhanced hydrophobicity of the purified antibody due to partial unfolding. Even loss of high affinity antibodies can occur, in case they cannot be eluted from the target at all without complete denaturation of the antibody. 2) Due to the nature of the mammalian immune system, the quality of the raised antibodies can be highly variable between different animals with respect to distribution of affinities and specificity, and also bleeds from a single animal taken consecutively over time inevitably are subjected to change with respect to distribution of affinity, specificity and also concentration of the target-specific antibody. Obviously, this generates problems of lot to lot variability of the antibody preparation itself, but as a consequence also brings about the risk of changed behavior of an immunoassay generated with different lots of purified polyclonal antibody.

Despite the disadvantages of polyclonal antibodies the commercially available immunoassays based thereon, such as in particular Elecsys® FT4 III (Material number 07976836190), show good analytical sensitivity and low cross reactivity with respect to cross reactants.

Even tough methods for generating monoclonal antibodies are available in the prior art (e.g., KÖHLER, G., MILSTEIN, C. Continuous cultures of fused cells secreting antibody of predefined specificity. Nature 256, 495-497 (1975). https://doi.org/10.1038/256495a0), it is not trivial to replace a polyclonal antibody in an immunoassay without losing signal dynamics and analyte specificity. Especially, when it comes to hapten analytes, such as L-T4, it is challenging to identify monoclonal antibodies that provide excellent kinetic features without showing undesired cross reactions with structurally closely related structures.

Accordingly, there is a high need to provide new monoclonal L-T4 antibodies with excellent kinetic features that can sufficiently discriminate L-T4 (e.g. fT4) from structurally related substances/derivates. Furthermore, there is a need to provide L-T4 (e.g. fT4) immunoassays based on monoclonal antibodies that show similar or even improved analytical performance and/or specificity regarding cross reactants than previously available and well-accepted assays based on polyclonal antibodies.

The above need is addressed by the present invention described herein.

In a first aspect, the present invention relates to a monoclonal antibody that specifically binds to L-thyroxine (T4). Among a set of 840 monoclonal antibodies binding to T4, the present inventors have surprisingly identified a family of three closely related antibodies with a very high sequence identity in the CDR sequences that share an extraordinary high association rate constant (kₐ) for the binding to T4. Such high association rate constant is important in high throughput immunoassays such as Elecsys® based immunoassays, in which there is typically only a very short time period for formation of antibody-antigen complexes available. Moreover, the antibodies of the invention are characterized by having a binding affinity with a K_{D} to fT4 in a subnanomolar range.

The family of antibodies provided herein is further characterized by a high specificity to fT4 and can especially discriminate fT4 from closely related compounds (see below).

Together these features make antibodies according to the first aspect of the invention a set of superior monoclonal antibodies, especially when used in competitive immunoassays for detecting the levels of fT4 in samples. With their kinetic characteristics, the monoclonal antibodies according to the first aspect of the invention can efficiently replace previously used polyclonal antibodies and thereby overcome all the disadvantages related to polyclonal antibodies. It is a surprising finding that the performance of a polyclonal antibody comprising a mixture of different antibodies can be mimicked so well by a single monoclonal antibody.

A crystal structure of the antibody 38F8 (the member of the identified antibody family with the best performance in the immunoassay experiments of Example 4) in complex with T4 revealed all amino acid residues directly interacting with T4. Strikingly, these amino acids are largely identical in the other two antibodies 7D4 and 7E10 of the identified antibody family. This high identity confirms that all three identified antibodies share at least largely identical interaction patterns. The few differences demonstrate that at the respective amino acid positions certain sequence variations do not dramatically affect antibody function, especially as far as it relates to the specificity for T4 and the association rate and K_{D} for the binding to T4.

The paratope of the antibody 38F8 identified by the co-crystal structure are shown in FIG. 5. Example 7 describes an in silico approach which defined possible amino acid substitutions based on sequence variations within the antibody family 38F8, 7E10 and 7D4 and a modelling approach.

As demonstrated by these analyses, the monoclonal antibody of the invention may comprise:
i) a heavy chain variable domain (VH) comprising V or A in position 33; Y in position 50; W in position 52; I in position 98; G, A or V in position 99; Y in position 100; and I in position 100b; and
ii) a light chain variable domain (VL) comprising amino acids H or Y in position 28; N or K in position 29; W in position 32; G or A in position 91; Y, W or F in position 92; S or T in position 93; Y or F in position 95b; N, S, T or Q in position 95c; and H in position 96.

The above-defined residues were identified as directly interacting with T4 in the co-crystal structure of 38F8 with L-T4. All the above positions of the amino acids are indicated according to the Kabat numbering scheme of the VH and VL, respectively.

In embodiments, the paratope of the monoclonal antibody for binding to T4 comprises the amino acids of the VH in positions 33, 50, 52, 98, 99, 100 and 100b and the amino acids of the VL in positions 28, 29, 32, 91, 92, 93, 95b, 95c and 96. Again, all positions are indicated according to the Kabat numbering scheme.

Methods for determining the amino acid residues belonging to the paratope are known in the art. The preferred method is crystallization of an antibody in complex with L-T4. An exemplary embodiment for such crystallization is provided in the appended Examples. The settings used and described in the appended Examples can also be applied to other T4 antibodies. Minor adaptations can be easily made by a skilled person based on such description.

Accordingly, herein provided is a monoclonal antibody that specifically binds to L-thyroxine (T4) comprising a paratope, said paratope comprising or consisting of:
i) a heavy chain variable domain (VH) comprising V or A in position 33; Y in position 50; W in position 52; I in position 98; G, A or V in position 99; Y in position 100; and I in position 100b; and
ii) a light chain variable domain (VL) comprising amino acids H or Y in position 28; N or K in position 29; W in position 32; G or A in position 91; Y, W or F in position 92; S or T in position 93; Y or F in position 95b; N, S, T or Q in position 95c; and H in position 96,
   wherein all positions are annoted according to Kabat nomenclature.

In particular embodiments, the monoclonal antibody comprises:
i) a heavy chain variable domain (VH) comprising V or A in position 33; Y in position 50; W in position 52; I in position 98; G, A or V in position 99; Y in position 100; and I in position 100b; and
ii) a light chain variable domain (VL) comprising amino acids H or Y in position 28; N or K in position 29; W in position 32; G or A in position 91; Y, W or F in position 92; S or T in position 93; Y or F in position 95b; N, S or T in position 95c; and H in position 96,
   wherein all positions are annotated according to Kabat nomenclature.

In other embodiments, the monoclonal antibody of the first aspect comprises
i) a heavy chain variable domain (VH) comprising V in position 33; Y in position 50; W in position 52; I in position 98; G in position 99; Y in position 100; and I in position 100b; and
ii) a light chain variable domain (VL) comprising amino acids H or Y in position 28; N or K in position 29; W in position 32; G in position 91; Y or W in position 92; S in position 93; Y in position 95b; N or S in position 95c; and H in position 96,
   wherein all positions are annotated according to Kabat nomenclature.

In a preferred embodiment, the monoclonal antibody of the first aspect comprises the residues found to directly interact with T4 in the antibody clone 38F8. Accordingly, the monoclonal antibody may comprise:
i) a heavy chain variable domain (VH) comprising V in position 33; Y in position 50; W in position 52; I in position 98; G in position 99; Y in position 100; and I in position 100b; and
ii) a light chain variable domain (VL) comprising amino acids H in position 28; N in position 29; W in position 32; G in position 91; Y in position 92; S in position 93; Y in position 95b; N in position 95c; and H in position 96,
   wherein all positions are annotated according to Kabat nomenclature.

In another preferred embodiment, the monoclonal antibody of the first aspect comprises the residues found in antibody 7D4 that correspond to the paratope residues in 38F8. Thus, in embodiments the monoclonal antibody according to the first as may comprise
i) a heavy chain variable domain (VH) comprising V in position 33; Y in position 50; W in position 52; I in position 98; G in position 99; Y in position 100; and I in position 100b; and
ii) a light chain variable domain (VL) comprising amino acids Y in position 28; K in position 29; W in position 32; G in position 91; W in position 92; S in position 93; Y in position 95b; S in position 95c; and H in position 96,
   wherein all positions are annotated according to Kabat nomenclature.

In another preferred embodiment, the monoclonal antibody of the first aspect comprises the residues as found in antibody 7E10 corresponding to the paratope residues of 38F8. Thus, the monoclonal antibody may comprise
i) a heavy chain variable domain (VH) comprising V in position 33; Y in position 50; W in position 52; I in position 98; G in position 99; Y in position 100; and I in position 100b; and
ii) a light chain variable domain (VL) comprising amino acids Y in position 28; K in position 29; W in position 32; G in position 91; W in position 92; S in position 93; Y in position 95b; N in position 95c; and H in position 96,
   wherein all positions are annotated according to Kabat nomenclature.

As elaborated in the appended Examples, there is in silico evidence that the amino acids in positions 34, 35, 51, 52a, 53, 97 and/or 100a of the VH according to Kabat numbering scheme and amino acids in positions 30, 31, 94, 95, 95a and/or 97 of the VL according to Kabat numbering scheme are of some relevance for positioning of the amino acids directly interacting with T4. Yet, as demonstrated by variations in these amino acid positions as found in the closely related antibodies 38F8, 7E10 and 7D4 or as evaluated in silico in the appended Examples, at least certain amino acid exchanges to other amino acids are acceptable at these positions (see also Table XX).

The VH of the monoclonal antibody of the invention may comprise M, L, I or V in position 34; N, S, T or Q in position 35; I, A, L or V in position 51, T or S in position 52a; R, G, D or K in position 53; H, A, R or K in position 97; and/or N, A or Q in position 100a, wherein the positions of the amino acids are indicated according to the Kabat numbering scheme.

In an embodiment, the VH of the monoclonal antibody of the invention comprises M, L, or I in position 34; N, S, or T in position 35; I, A or L in position 51, T or S in position 52a; R, G, D or K in position 53; H, A, R or K in position 97; and/or N, A or Q in position 100a, wherein the positions of the amino acids are indicated according to the Kabat numbering scheme.

In a particular embodiment, the VH of the monoclonal antibody of the invention comprises M or L in position 34; N in position 35; I in position 51, T in position 52a; R, G or D in position 53; H or A in position 97; and/or N or A in position 100a, wherein the positions of the amino acids are indicated according to the Kabat numbering scheme.

In an even more specific embodiment, the VH of the monoclonal antibody of the invention comprises M in position 34; N in position 35; I in position 51, T in position 52a; R in position 53; H in position 97; and/or N in position 100a, wherein the positions of the amino acids are indicated according to the Kabat numbering scheme.

In an even more specific embodiment, the VH of the monoclonal antibody of the invention comprises M in position 34; N in position 35; I in position 51, T in position 52a; R in position 53; H in position 97; and N in position 100a, wherein the positions of the amino acids are indicated according to the Kabat numbering scheme. These amino acids can be found, for example, in the VH of antibody 38F8.

In another specific embodiment, the VH of the monoclonal antibody of the invention comprises L in position 34; N in position 35; I in position 51, T in position 52a; G in position 53; A in position 97; and A in position 100a, wherein the positions of the amino acids are indicated according to the Kabat numbering scheme. These amino acids can be found, for example, in the VH of antibody 7D4.

In another specific embodiment, the VH of the monoclonal antibody of the invention comprises M in position 34; N in position 35; I in position 51, T in position 52a; D in position 53; A in position 97; and A in position 100a, wherein the positions of the amino acids are indicated according to the Kabat numbering scheme. These amino acids can be found, for example, in the VH of antibody 7E10.

The VL of the monoclonal antibody of the invention may comprise N, Q, S or T in position 30; A, N or V in position 31; G, A or S in position 94; S, G, N or Q in position 95; T, S or G in position 95a; and/or V, A, I or L in position 97, wherein the positions of the amino acids are indicated according to the Kabat numbering scheme.

In an embodiment, the VL of the monoclonal antibody of the invention comprises N in position 30; A or N in position 31; G, A or S in position 94; S, G or N in position 95; T, S or G in position 95a; and/or V or A in position 97, wherein the positions of the amino acids are indicated according to the Kabat numbering scheme.

In an even more specific embodiment, the VL of the monoclonal antibody of the invention comprises N in position 30; A in position 31; G in position 94; S in position 95; T in position 95a; and/or V in position 97, wherein the positions of the amino acids are indicated according to the Kabat numbering scheme. These amino acids can be found, for example, in the VL of antibody 38F8.

In another specific embodiment, the VL of the monoclonal antibody of the invention comprises N in position 30; N in position 31; A in position 94; G in position 95; G in position 95a; and/or A in position 97, wherein the positions of the amino acids are indicated according to the Kabat numbering scheme. These amino acids can be found, for example, in the VL of antibody 7D4.

In another specific embodiment, the VL of the monoclonal antibody of the invention comprises N in position 30; N in position 31; S in position 94; N in position 95; S in position 95a; and/or A in position 97, wherein the positions of the amino acids are indicated according to the Kabat numbering scheme. These amino acids can be found, for example, in the VL of antibody 7E10.

As discussed in the appended Examples, amino acids in the CDRs of the novel antibodies directed against T4 that are (i) neither interacting with T4 according to the crystal structure (ii) nor were identified to be of relevance for positioning the interacting residues can be substituted with other amino acids. These amino acid positions include all amino acids in the CDRs (preferably as defined by Kabat numbering scheme) other than the amino acids residues in positions 33, 50, 52, 98, 99, 100, 100b, 34, 35, 51, 52a, 53, 97 and 100a of the VH and in positions 28, 29, 32, 91, 92, 93, 95b, 95c, 96, 30, 31, 94, 95, 95a and 97 of the VL according to Kabat numbering scheme. In embodiments, the following amino acids positions according to Kabat numbering scheme can be substituted by other amino acids:
i) positions 31, 32, 54 to 65, 95, 96, 100c, 101 and 102 of the VH; and
ii) positions 24, 25, 26, 27, 27a, 27b, 33, 34, 50-56, 89 and 90 of the VL.

The VH of the antibodies of the invention may comprises amino acids in positions 31 and 32 according to the Kabat numbering scheme which are independently selected from any amino acids other than proline (e.g. any naturally occurring amino acid other than proline), in embodiments any amino acids other than proline or cysteine (e.g. any naturally occurring amino acid other than P or C). In embodiments, the VH comprises S, R or a conservative substitution thereof in position 31 according to the Kabat numbering scheme; and/or N or a conservative substitution thereof in position 32 according to the Kabat numbering scheme. For example, the amino acid in position 31 of the VH according to Kabat numbering may be selected from S or R. The amino acid in position 32 may be N. In specific embodiments, the VH comprises S or R in position 31 according to the Kabat numbering scheme; and/or N in position 32 according to the Kabat numbering scheme.

In embodiments, the CDR-H1 of the VH of the monoclonal antibody of the invention comprises positions 31 to 35 according to Kabat numbering scheme. In even more preferred embodiments, the CDR-H1 of the VH consists of positions 31 to 35 according to Kabat numbering scheme.

The VH of the monoclonal antibody of the invention preferably comprises amino acids in positions 54 to 65 according to the Kabat numbering scheme. These amino acids in positions 54 to 65 may be each individually selected from any amino acids other than proline (e.g. may be any naturally occurring amino acid other than proline), in embodiments may be selected from any amino acids other than proline or cysteine (e.g. may be any naturally occurring amino acid other than P or C). In embodiments, the amino acids of positions 54 to 65 according to the Kabat numbering scheme may have the amino acid sequence SGNTYYASWAKG (SEQ ID NO: 2) or a variant thereof having 12 or less, 11 or less, 10 or less, 9 or less, 8 or less, 7 or less, 6 or less, 5 or less, 4 or less, 3 or less, 2 or less, or 1 or less amino acid exchanges. Preferably, none of the positions 54 to 65 is proline or in embodiments proline and cysteine. In a preferred embodiment the amino acids of positions 54 to 65 according to the Kabat numbering scheme may have the amino acid sequence SGNTYYASWAKG (SEQ ID NO: 2) or a variant thereof having 2 or less amino acid exchanges, wherein none of the positions 54 to 65 is proline or in embodiments proline and cysteine. In embodiments, all the amino acid exchanges in SEQ ID NO:2 may be each individually conservative amino acid exchanges. In specific embodiments, the amino acid sequence in positions 54 to 65 according to the Kabat numbering scheme may be selected from the group consisting of: SGNTYYASWAKG (SEQ ID NO: 1), SGNTYYATWAKG (SEQ ID NO: 2) or SGSTYYATWAKG (SEQ ID NO:3). These are the corresponding sequences found in antibodies 38F8, 7D4 and 7E10.

In embodiments, the CDR-H2 of the VH of the monoclonal antibody of the invention comprises positions 50, 51, 52, 52a and 53 to 65 (i.e. 53, 54, 55, 56, 57, 58, 59, 60, 61, 62, 63, 63 and 65) according to the Kabat numbering scheme. In preferred embodiments, the CDR-H2 of the VH of the monoclonal antibody of the invention consists of positions 50, 51, 52, 52a and 53 to 65 (i.e. 53, 54, 55, 56, 57, 58, 59, 60, 61, 62, 63, 63 and 65) according to the Kabat numbering scheme.

In embodiments, the VH comprises amino acids in positions 95, 96, 100c, 101 and 102 according to the Kabat numbering scheme The amino acids in positions 95, 96, 100c, 101 and 102 are preferably individually selected from any amino acids (e.g. any naturally occurring amino acid) other than proline or preferably other than proline and cysteine. In embodiments, the amino acid in positions in positions 95, 96, 100c, 101 and 102 of the VH are selected as follows: G or a conservative substitution thereof in position 95; L or a conservative substitution thereof in position 96; F or a conservative substitution thereof in position 100c; N or a conservative substitution thereof in position 101; and F or a conservative substitution thereof in position 102, wherein the positions of the amino acids are indicated according to the Kabat numbering scheme. In specific embodiments, the amino acid in positions in positions 95, 96, 100c, 101 and 102 of the VH are selected as follows: G in position 95; L in position 96; F in position 100c; N in position 101; and F in position 102.

In embodiments, the CDR-H3 of VH of the monoclonal antibody of the invention comprises positions 95 to 100 (i.e. 95, 96, 97, 98, 99 and 100), 100a, 100b, 100c, 101 and 102 according to the Kabat numbering scheme. In preferred embodiments, the CDR-H3 of the VH of the monoclonal antibody of the invention consists of positions 95 to 100 (i.e. 95, 96, 97, 98, 99 and 100), 100a, 100b, 100c, 101 and 102 according to the Kabat numbering scheme.

The VL of the monoclonal antibody of the invention may comprise amino acids in positions 24, 25, 26, 27, 27a, 27b, 33 and 34 according to the Kabat numbering scheme. In embodiments, the VL of the monoclonal antibody of the invention may comprise any amino acids (e.g. any naturally occurring amino acid) other than proline in positions 24, 25, 26, 27, 27a, 27b, 33 and 34 according to the Kabat numbering scheme. In embodiments, the VL of the monoclonal antibody of the invention may comprise any amino acids (e.g. any naturally occurring amino acid) other than proline and cysteine in positions 24, 25, 26, 27, 27a, 27b, 33 and 34 according to the Kabat numbering scheme.

In embodiments, the VL of the monoclonal antibody of the invention comprises Q or a conservative substitution thereof in position 24; S or a conservative substitution thereof in position 25; S or a conservative substitution thereof in position 26; Q or a conservative substitution thereof in position 27; S or a conservative substitution thereof in position 27a; V or a conservative substitution thereof in position 27b; C, L or a conservative substitution thereof in position 33; and/or S or a conservative substitution thereof in position 34, wherein the positions of the amino acids are indicated according to the Kabat numbering scheme. In embodiments, the VL of the monoclonal antibody of the invention comprises Q or a conservative substitution thereof in position 24; S or a conservative substitution thereof in position 25; S or a conservative substitution thereof in position 26; Q or a conservative substitution thereof in position 27; S or a conservative substitution thereof in position 27a; V or a conservative substitution thereof in position 27b; C, L or a conservative substitution thereof in position 33; and S or a conservative substitution thereof in position 34, wherein the positions of the amino acids are indicated according to the Kabat numbering scheme. In specific embodiments, VL of the monoclonal antibody of the invention comprises Q in position 24; S in position 25; S in position 26; Q in position 27; S in position 27a; V in position 27b; C or L in position 33; and/or S in position 34, wherein all positions are indicated according to the Kabat numbering scheme. In other specific embodiments, VL of the monoclonal antibody of the invention comprises Q in position 24; S in position 25; S in position 26; Q in position 27; S in position 27a; V in position 27b; C or L in position 33; and S in position 34, wherein all positions are indicated according to the Kabat numbering scheme.

In embodiments, the CDR-L1 of VL of the monoclonal antibody of the invention comprises positions 24, 25, 26, 27, 27a, 27b, 28, 29, 30, 31, 32, 33 and 34 according to the Kabat numbering scheme. In preferred embodiments, the CDR-L1 of VL of the monoclonal antibody of the invention consists of positions 24, 25, 26, 27, 27a, 27b, 28, 29, 30, 31, 32, 33 and 34 according to the Kabat numbering scheme.

The VL of the monoclonal antibody of the invention may comprise amino acids in positions 50, 51, 52, 53, 54, 55 and 56 according to the Kabat numbering scheme. In embodiments, the VL of the monoclonal antibody of the invention may comprise any amino acids (e.g. any naturally occurring amino acid) other than proline in positions 50, 51, 52, 53, 54, 55 and 56 according to the Kabat numbering scheme. In embodiments, the VL of the monoclonal antibody of the invention may comprise any amino acids (e.g. any naturally occurring amino acid) other than proline and cysteine in positions 50, 51, 52, 53, 54, 55 and 56 according to the Kabat numbering scheme.

In embodiments, the VL of the monoclonal antibody of the invention comprises G or a conservative substitution thereof in position 50; A or a conservative substitution thereof in position 51; S or a conservative substitution thereof in position 52; T or a conservative substitution thereof in position 53; L or a conservative substitution thereof in position 54; T, A or a conservative substitution thereof in position 55; and/or C, S or a conservative substitution thereof in position 56, wherein the positions of the amino acids are indicated according to the Kabat numbering scheme. In embodiments, the VL of the monoclonal antibody of the invention comprises G or a conservative substitution thereof in position 50; A or a conservative substitution thereof in position 51; S or a conservative substitution thereof in position 52; T or a conservative substitution thereof in position 53; L or a conservative substitution thereof in position 54; T, A or a conservative substitution thereof in position 55; and C, S or a conservative substitution thereof in position 56, wherein the positions of the amino acids are indicated according to the Kabat numbering scheme. In specific embodiments the VL of the monoclonal antibody of the invention comprises GASTLTS (SEQ ID NO: 4) or GASTLAS (SEQ ID NO: 5) in positions 50 to 56 according to the Kabat numbering scheme.

In embodiments, the CDR-L2 of VL of the monoclonal antibody of the invention comprises positions 50, 51, 52, 53, 54, 55 and 56 according to the Kabat numbering scheme. In preferred embodiments, the CDR-L2 of VL of the monoclonal antibody of the invention consists of positions 50, 51, 52, 53, 54, 55 and 56 according to the Kabat numbering scheme.

The VL of the monoclonal antibody of the invention may comprise amino acids in positions 89 and 90 according to the Kabat numbering scheme. In embodiments, the VL of the monoclonal antibody of the invention may comprise any amino acids (e.g. any naturally occurring amino acid) other than proline in positions 89 and 90 according to the Kabat numbering scheme. In embodiments, the VL of the monoclonal antibody of the invention may comprise any amino acids (e.g. any naturally occurring amino acid) other than proline and cysteine in positions 89 and 90 according to the Kabat numbering scheme.

In embodiments, the VL of the monoclonal antibody of the invention comprises A or a conservative substitution thereof in position 89; and/or G or a conservative substitution thereof in position 90, wherein the positions of the amino acids are indicated according to the Kabat numbering scheme. In embodiments, the VL of the monoclonal antibody of the invention comprises A or a conservative substitution thereof in position 89; and G or a conservative substitution thereof in position 90, wherein the positions of the amino acids are indicated according to the Kabat numbering scheme. In specific embodiments, the VL of the monoclonal antibody of the invention comprises A in position 89; and/or G in position 90. In specific embodiments, the VL of the monoclonal antibody of the invention comprises A in position 89 and G in position 90 according to Kabat numbering scheme.

In embodiments, the CDR-L3 of VL of the monoclonal antibody of the invention comprises positions 89, 90, 91, 92, 93, 94, 95, 95a, 95b, 95c, 96 and 97 according to the Kabat numbering scheme. In preferred embodiments, the CDR-L3 of VL of the monoclonal antibody of the invention consists of positions 89, 90, 91, 92, 93, 94, 95, 95a, 95b, 95c, 96 and 97 according to the Kabat numbering scheme.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 4 or less, 3 or less, 2 or less, or 1 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 15 or less, 14 or less, 13 or less, 12 or less, 11 or less, 10 or less, 9 or less, 8 or less, 7 or less, 6 or less, 5 or less, 4 or less, 3 or less, 2 or less, 1 or less positions selected from positions 2 and 4 to 17 of SEQ ID NO: 7, and (c) a CDR-H3 having the amino acid sequence of SEQ ID NO: 8 or a variant thereof having amino acid substitutions (preferably conservative or highly conservative amino acid substitutions) in 11 or less, 10 or less, 9 or less, 8 or less, 7 or less, 6 or less, 5 or less, 4 or less, 3 or less, 2 or less, or 1 or less positions selected from positions 1 to 3, 7, and 9 to 11 of SEQ ID NO: 8; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 10 or less, 9 or less, 8 or less, 7 or less, 6 or less, 5 or less, 4 or less, 3 or less, 2 or less, or 1 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions (preferably conservative or highly conservative amino acid substitutions) in 6 or less, 5 or less, 4 or less, 3 or less, 2 or less, or 1 or less positions selected from positions 1, 2, 6 to 8 and 12 of SEQ ID NO: 10.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 4 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 4 or less positions selected from positions 2 and 4 to 17 of SEQ ID NO: 7, and (c) a CDR-H3 having the amino acid sequence of SEQ ID NO: 8 or a variant thereof having amino acid substitutions (preferably conservative or highly conservative amino acid substitutions) in 4 or less positions selected from positions 1 to 3, 7, and 9 to 11 of SEQ ID NO: 8; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 4 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions (preferably conservative or highly conservative amino acid substitutions) in 4 or less positions selected from positions 1, 2, 6 to 8 and 12 of SEQ ID NO: 10.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having an amino acid substitution (preferably a conservative or highly conservative amino acid substitution) in 1 or less position selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7, and (c) a CDR-H3 having the amino acid sequence of SEQ ID NO: 8 or a variant thereof having amino acid substitutions (preferably conservative or highly conservative amino acid substitutions) in 2 or less positions selected from positions 1 to 3, 7, and 9 to 11 of SEQ ID NO: 8; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 10 or a variant thereof having an amino acid substitution (preferably a conservative or highly conservative amino acid substitution) in 1 or less position selected from positions 1, 2, 6 to 8 and 12 of SEQ ID NO: 10.

In a specific embodiment, the monoclonal antibody may comprise
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 2 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7; and (c) a CDR-H3 having the amino acid sequence of SEQ ID NO: 8 or a variant thereof having amino acid substitutions (preferably conservative or highly conservative amino acid substitutions) in 2 or less positions selected from positions 1 to 3, 7, and 9 to 11 of SEQ ID NO: 8; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions (preferably conservative or highly conservative amino acid substitutions) in 3 or less positions selected from positions 1, 2, 6 to 8 and 12 of SEQ ID NO: 10.

In an even more specific embodiment, the monoclonal antibody may comprise
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 2 or less positions selected from positions 1 and 4 of SEQ ID NO: 6; (b) a CDR-H2 having the amino acid sequence of SEQ ID NO:7 or a variant thereof having amino acid substitutions (preferably conservative or highly conservative amino acid substitutions) in 3 or less positions selected from positions 5, 8 and 13 of SEQ ID NO: 7, and (c) a CDR-H3 having the amino acid sequence of SEQ ID NO: 8 or a variant thereof having amino acid substitutions (preferably conservative or highly conservative amino acid substitutions) in 2 or less positions selected from positions 3 and 7 of SEQ ID NO: 8; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 8 and 10 of SEQ ID NO: 9 and (f) a CDR-L3 having the amino acid sequence of SEQ ID NO: 10 or a variant thereof having amino acid substitutions (preferably conservative or highly conservative amino acid substitutions) in 3 or less positions selected from positions 4, 5 and 6 of SEQ ID NO: 10.

In embodiments, the variant of SEQ ID NO: 6 has a M, L, I or V in position 4 and a N, S, T or Q in position 5; the variant of SEQ ID NO: 7 has a I, A, L or V in position 2; a T or S in position 4; a R, G, D or K in position 5; the variant of SEQ ID NO: 8 has a H, A, R or K in position 3 and a N, A or Q in position 7; the variant of SEQ ID NO: 9 has a N, Q, S or T in position 9 and A, N or V in position 10; and/or the variant of SEQ ID NO:10 has a G, A or S in position 6; a S, G, N or Q in position 7; T, S or G in position 8; and V, A, I or L in position 12.

In specific embodiments, the variant of SEQ ID NO: 6 has a M, L, or I in position 4 and a N, S or T in position 5; the variant of SEQ ID NO: 7 has a I, A or L in position 2; a T or S in position 4; a R, G, D or K in position 5; the variant of SEQ ID NO: 8 has a H, A, R or K in position 3 and a N, A or Q in position 7; the variant of SEQ ID NO: 9 has a N, Q, S or T in position 9 and A, N or V in position 10; and/or the variant of SEQ ID NO:10 has a G, A or S in position 6; a S, G or N in position 7; T, S or G in position 8; and V or A in position 12.

In even more specific embodiments, the variant of SEQ ID NO: 6 has a M or L in position 4 and a N in position 5; the variant of SEQ ID NO: 7 has a I in position 2; a T in position 4; a R, G or D in position 5; the variant of SEQ ID NO: 8 has a H or A in position 3 and a N or A in position 7; the variant of SEQ ID NO: 9 has a N in position 9 and A or N in position 10; and/or the variant of SEQ ID NO:10 has a G, A or S in position 6; a S, G or N in position 7; T, S or G in position 8; and/or V or A in position 12.

In a preferred embodiment, the variants of SEQ ID NOs: 6 to 10 have compared to SEQ ID NOs: 6 to 10 amino acid substitutions (preferably conservative or highly conservative amino acid substitutions) at 12 or less positions.

Also provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 4 or less, 3 or less, 2 or less, or 1 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 15 or less, 14 or less, 13 or less, 12 or less, 11 or less, 10 or less, 9 or less, 8 or less, 7 or less, 6 or less, 5 or less, 4 or less, 3 or less, 2 or less, 1 or less positions selected from positions 2 and 4 to 17 of SEQ ID NO: 7, and (c) a CDR-H3 having the amino acid sequence of SEQ ID NO: 13 or a variant thereof having amino acid substitutions (preferably conservative or highly conservative amino acid substitutions) in 11 or less, 10 or less, 9 or less, 8 or less, 7 or less, 6 or less, 5 or less, 4 or less, 3 or less, 2 or less, or 1 or less positions selected from positions 1 to 3, 7, and 9 to 11 of SEQ ID NO: 13; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 10 or less, 9 or less, 8 or less, 7 or less, 6 or less, 5 or less, 4 or less, 3 or less, 2 or less, or 1 or less positions selected from positions 1 to 6, 9, 10, 12 and 13 of SEQ ID NO: 9 and (f) a CDR-L3 having the amino acid sequence of SEQ ID NO: 16 or a variant thereof having amino acid substitutions (preferably conservative or highly conservative amino acid substitutions) in 6 or less, 5 or less, 4 or less, 3 or less, 2 or less, or 1 or less positions selected from positions 1, 2, 6 to 8 and 12 of SEQ ID NO: 16.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 4 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 4 or less positions selected from positions 2 and 4 to 17 of SEQ ID NO: 7, and (c) a CDR-H3 having the amino acid sequence of SEQ ID NO: 13 or a variant thereof having amino acid substitutions (preferably conservative or highly conservative amino acid substitutions) in 4 or less positions selected from positions 1 to 3, 7, and 9 to 11 of SEQ ID NO: 13; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 4 or less positions selected from positions 1 to 6, 9, 10, 12 and 13 of SEQ ID NO: 9 and (f) a CDR-L3 having the amino acid sequence of SEQ ID NO: 16 or a variant thereof having amino acid substitutions (preferably conservative or highly conservative amino acid substitutions) in 4 or less positions selected from positions 1, 2, 6 to 8 and 12 of SEQ ID NO: 16.

In one aspect, provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 6 or a variant thereof having an amino acid substitution (preferably a conservative or highly conservative amino acid substitution) in 1 or less of the position selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7, and (c) a CDR-H3 having the amino acid sequence of SEQ ID NO: 13 or a variant thereof having amino acid substitutions (preferably conservative or highly conservative amino acid substitutions) in 2 or less positions selected from positions 1 to 3, 7, and 9 to 11 of SEQ ID NO: 13; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 16 or a variant thereof having an amino acid substitution (preferably a conservative or highly conservative amino acid substitution) in 1 or less position selected from positions 1, 2, 6 to 8 and 12 of SEQ ID NO: 16.

In a specific embodiment, the monoclonal antibody may comprise
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 2 or less positions selected from positions 1, 2, 4, and 5 of SEQ ID NO: 6; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7; and (c) a CDR-H3 having the amino acid sequence of SEQ ID NO: 13 or a variant thereof having amino acid substitutions (preferably conservative or highly conservative amino acid substitutions) in 2 or less positions selected from positions 1 to 3, 7, and 9 to 11 of SEQ ID NO: 13; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 16 or a variant thereof having amino acid substitutions (preferably conservative or highly conservative amino acid substitutions) in 3 or less positions selected from positions 1, 2, 6 to 8 and 12 of SEQ ID NO: 16.

In an even more specific embodiment, the monoclonal antibody may comprise
i) a heavy chain variable domain (VH) comprising (a) a CDR-H1 having the amino acid sequence of SEQ ID NO: 6 or a variant thereof having amino acid substitutions (preferably conservative or highly conservative amino acid substitutions) in 2 or less positions selected from positions 1 and 4 of SEQ ID NO: 6; (b) a CDR-H2 having the amino acid sequence of SEQ ID NO:7 or a variant thereof having amino acid substitutions (preferably conservative or highly conservative amino acid substitutions) in 3 or less positions selected from positions 5, 8 and 13 of SEQ ID NO: 7, and (c) a CDR-H3 having the amino acid sequence of SEQ ID NO: 13 or a variant thereof having amino acid substitutions (preferably conservative or highly conservative amino acid substitutions) in 2 or less positions selected from positions 3 and 7 of SEQ ID NO: 13; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 8 and 10 of SEQ ID NO: 9 and (f) a CDR-L3 having the amino acid sequence of SEQ ID NO: 16 or a variant thereof having amino acid substitutions (preferably conservative or highly conservative amino acid substitutions) in 3 or less positions selected from positions 4, 5 and 6 of SEQ ID NO: 16.

In embodiments, the variant of SEQ ID NO: 6 has a M, L, I or V in position 4 and a N, S, T or Q in position 5; the variant of SEQ ID NO: 7 has a I, A, L or V in position 2; a T or S in position 4; a R, G, D or K in position 5; the variant of SEQ ID NO: 13 has a H, A, R or K in position 3 and a N, A or Q in position 7; the variant of SEQ ID NO: 9 has a N, Q, S or T in position 9 and A, N or V in position 10; and/or the variant of SEQ ID NO:16 has a G, A or S in position 6; a S, G, N or Q in position 7; T, S or G in position 8; and V, A, I or L in position 12.

In specific embodiments, the variant of SEQ ID NO: 6 has a M, L, or I in position 4 and a N, S or T in position 5; the variant of SEQ ID NO: 7 has a I, A or L in position 2; a T or S in position 4; a R, G, D or K in position 5; the variant of SEQ ID NO: 13 has a H, A, R or K in position 3 and a N, A or Q in position 7; the variant of SEQ ID NO: 9 has a N, Q, S or T in position 9 and A, N or V in position 10; and/or the variant of SEQ ID NO:16 has a G, A or S in position 6; a S, G or N in position 7; T, S or G in position 8; and V or A in position 12.

In even more specific embodiments, the variant of SEQ ID NO: 6 has a M or L in position 4 and a N in position 5; the variant of SEQ ID NO: 7 has a I in position 2; a T in position 4; a R, G or D in position 5; the variant of SEQ ID NO: 13 has a H or A in position 3 and a N or A in position 7; the variant of SEQ ID NO: 9 has a N in position 9 and A or N in position 10; and/or the variant of SEQ ID NO:16 has a G, A or S in position 6; a S, G or N in position 7; T, S or G in position 8; and/or V or A in position 12.

In a preferred embodiment, the variants of SEQ ID NOs: 6, 7, 13, 9 and 16 have compared to SEQ ID NOs: 6, 7, 13, 9 and 16, respectively, amino acid substitutions (preferably conservative or highly conservative amino acid substitutions) at 12 or less positions.

Also provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 4 or less, 3 or less, 2 or less, or 1 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 15 or less, 14 or less, 13 or less, 12 or less, 11 or less, 10 or less, 9 or less, 8 or less, 7 or less, 6 or less, 5 or less, 4 or less, 3 or less, 2 or less, 1 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 11 or less, 10 or less, 9 or less, 8 or less, 7 or less, 6 or less, 5 or less, 4 or less, 3 or less, 2 or less, or 1 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 10 or less, 9 or less, 8 or less, 7 or less, 6 or less, 5 or less, 4 or less, 3 or less, 2 or less, or 1 or less positions selected from positions 1 to 6, 9, 10, 12 and 13 of SEQ ID NO: 9 and (f) a CDR-L3 having the amino acid sequence of SEQ ID NO: 17 or a variant thereof having amino acid substitutions (preferably conservative or highly conservative amino acid substitutions) in 6 or less, 5 or less, 4 or less, 3 or less, 2 or less, or 1 or less positions selected from positions 1, 2, 6 to 8 and 12 of SEQ ID NO: 17.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 4 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 4 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 4 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 4 or less positions selected from positions 1 to 6, 9, 10, 12 and 13 of SEQ ID NO: 9 and (f) a CDR-L3 having the amino acid sequence of SEQ ID NO: 17 or a variant thereof having amino acid substitutions (preferably conservative or highly conservative amino acid substitutions) in 4 or less positions selected from positions 1, 2, 6 to 8 and 12 of SEQ ID NO: 17.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having an amino acid substitution (preferably a conservative or highly conservative amino acid substitution) in 1 or less of the position selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 2 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 16 or a variant thereof having an amino acid substitution (preferably a conservative or highly conservative amino acid substitution) in 1 or less position selected from positions 1, 2, 6 to 8 and 12 of SEQ ID NO: 17.

In a specific embodiment, the monoclonal antibody may comprise
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 2 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7; and (c) a CDR-H3 having the amino acid sequence of SEQ ID NO: 14 or a variant thereof having amino acid substitutions (preferably conservative or highly conservative amino acid substitutions) in 2 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 1 to 6, 9, 10, 12 and 13 of SEQ ID NO: 9 and (f) a CDR-L3 having the amino acid sequence of SEQ ID NO: 17 or a variant thereof having amino acid substitutions (preferably conservative or highly conservative amino acid substitutions) in 3 or less positions selected from positions 1, 2, 6 to 8 and 12 of SEQ ID NO: 17.

In an even more specific embodiment, the monoclonal antibody may comprise
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 2 or less positions selected from positions 1 and 4 of SEQ ID NO: 12; (b) a CDR-H2 having the amino acid sequence of SEQ ID NO:7 or a variant thereof having amino acid substitutions (preferably conservative or highly conservative amino acid substitutions) in 3 or less positions selected from positions 5, 8 and 13 of SEQ ID NO: 7, and (c) a CDR-H3 having the amino acid sequence of SEQ ID NO: 14 or a variant thereof having amino acid substitutions (preferably conservative or highly conservative amino acid substitutions) in 2 or less positions selected from positions 3 and 7 of SEQ ID NO: 14; and
ii) a light chain variable domain (VL) comprising (d) a CDR-L1 having the amino acid sequence of SEQ ID NO: 9 or a variant thereof having amino acid substitutions (preferably conservative or highly conservative amino acid substitutions) in 2 or less positions selected from positions 8 and 10 of SEQ ID NO: 9 and (f) a CDR-L3 having the amino acid sequence of SEQ ID NO: 17 or a variant thereof having amino acid substitutions (preferably conservative or highly conservative amino acid substitutions) in 3 or less positions selected from positions 4, 5 and 6 of SEQ ID NO: 17.

In embodiments, the variant of SEQ ID NO: 12 has a M, L, I or V in position 4 and a N, S, T or Q in position 5; the variant of SEQ ID NO: 7 has a I, A, L or V in position 2; a T or S in position 4; a R, G, D or K in position 5; the variant of SEQ ID NO: 14 has a H, A, R or K in position 3 and a N, A or Q in position 7; the variant of SEQ ID NO: 9 has a N, Q, S or T in position 9 and A, N or V in position 10; and/or the variant of SEQ ID NO:17 has a G, A or S in position 6; a S, G, N or Q in position 7; T, S or G in position 8; and V, A, I or L in position 12.

In specific embodiments, the variant of SEQ ID NO: 12 has a M, L, or I in position 4 and a N, S or T in position 5; the variant of SEQ ID NO: 7 has a I, A or L in position 2; a T or S in position 4; a R, G, D or K in position 5; the variant of SEQ ID NO: 14 has a H, A, R or K in position 3 and a N, A or Q in position 7; the variant of SEQ ID NO: 9 has a N, Q, S or T in position 9 and A, N or V in position 10; and/or the variant of SEQ ID NO:17 has a G, A or S in position 6; a S, G or N in position 7; T, S or G in position 8; and V or A in position 12.

In even more specific embodiments, the variant of SEQ ID NO: 12 has a M or L in position 4 and a N in position 5; the variant of SEQ ID NO: 7 has a I in position 2; a T in position 4; a R, G or D in position 5; the variant of SEQ ID NO: 14 has a H or A in position 3 and a N or A in position 7; the variant of SEQ ID NO: 9 has a N in position 9 and A or N in position 10; and/or the variant of SEQ ID NO:17 has a G, A or S in position 6; a S, G or N in position 7; T, S or G in position 8; and/or V or A in position 12.

In a preferred embodiment, the variants of SEQ ID NOs: 12, 7, 14, 9 and 17 have compared to SEQ ID NOs: 12, 7, 14, 9 and 17, respectively, amino acid substitutions (preferably conservative or highly conservative amino acid substitutions) at 12 or less positions.

Also provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 4 or less, 3 or less, 2 or less, or 1 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 15 or less, 14 or less, 13 or less, 12 or less, 11 or less, 10 or less, 9 or less, 8 or less, 7 or less, 6 or less, 5 or less, 4 or less, 3 or less, 2 or less, 1 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 11 or less, 10 or less, 9 or less, 8 or less, 7 or less, 6 or less, 5 or less, 4 or less, 3 or less, 2 or less, or 1 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 15 or a variant thereof having amino acid substitutions (preferably conservative or highly conservative amino acid substitutions) in 10 or less, 9 or less, 8 or less, 7 or less, 6 or less, 5 or less, 4 or less, 3 or less, 2 or less, or 1 or less positions selected from positions 1 to 6, 9, 10, 12 and 13 of SEQ ID NO: 15 and (f) a CDR-L3 having the amino acid sequence of SEQ ID NO: 18 or a variant thereof having amino acid substitutions (preferably conservative or highly conservative amino acid substitutions) in 6 or less, 5 or less, 4 or less, 3 or less, 2 or less, or 1 or less positions selected from positions 1, 2, 6 to 8 and 12 of SEQ ID NO: 18.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 4 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 4 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 4 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 15 or a variant thereof having amino acid substitutions (preferably conservative or highly conservative amino acid substitutions) in 4 or less positions selected from positions 1 to 6, 9, 10, 12 and 13 of SEQ ID NO: 15 and (f) a CDR-L3 having the amino acid sequence of SEQ ID NO: 18 or a variant thereof having amino acid substitutions (preferably conservative or highly conservative amino acid substitutions) in 4 or less positions selected from positions 1, 2, 6 to 8 and 12 of SEQ ID NO: 18.

In one aspect provided herein is a monoclonal antibody specifically binding to T4, wherein said monoclonal antibody comprises:
i) a heavy chain variable domain (VH) comprising (a) a CDR-H1 having the amino acid sequence of SEQ ID NO: 12 or a variant thereof having an amino acid substitution (preferably a conservative or highly conservative amino acid substitution) in 1 or less of the position selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7, and (c) a CDR-H3 having the amino acid sequence of SEQ ID NO: 14 or a variant thereof having amino acid substitutions (preferably conservative or highly conservative amino acid substitutions) in 2 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 15 or a variant thereof having amino acid substitutions (preferably conservative or highly conservative amino acid substitutions) in 2 or less positions selected from positions 1 to 6, 9, 10, 12 and 13 of SEQ ID NO: 15 and (f) a CDR-L3 having the amino acid sequence of SEQ ID NO: 18 or a variant thereof having an amino acid substitution (preferably a conservative or highly conservative amino acid substitution) in 1 or less position selected from positions 1, 2, 6 to 8 and 12 of SEQ ID NO: 18.

In a specific embodiment, the monoclonal antibody may comprise
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 2 or less positions selected from positions 1, 2, 4, and 5 of SEQ ID NO: 12; (b) a CDR-H2 having the amino acid sequence of SEQ ID NO: 7 or a variant thereof having amino acid substitutions (preferably conservative or highly conservative amino acid substitutions) in 3 or less positions selected from positions 2 and 4 to 17 of SEQ ID NO: 7; and (c) a CDR-H3 having the amino acid sequence of SEQ ID NO: 14 or a variant thereof having amino acid substitutions (preferably conservative or highly conservative amino acid substitutions) in 2 or less positions selected from positions 1 to 3, 7, and 9 to 11 of SEQ ID NO: 14; and
ii) a light chain variable domain (VL) comprising (d) a CDR-L1 having the amino acid sequence of SEQ ID NO: 15 or a variant thereof having amino acid substitutions (preferably conservative or highly conservative amino acid substitutions) in 2 or less positions selected from positions 1 to 6, 9, 10, 12 and 13 of SEQ ID NO: 15 and (f) a CDR-L3 having the amino acid sequence of SEQ ID NO: 18 or a variant thereof having amino acid substitutions (preferably conservative or highly conservative amino acid substitutions) in 3 or less positions selected from positions 1, 2, 6 to 8 and 12 of SEQ ID NO: 18.

In an even more specific embodiment, the monoclonal antibody may comprise
i) a heavy chain variable domain (VH) comprising (a) a CDR-H1 having the amino acid sequence of SEQ ID NO: 12 or a variant thereof having amino acid substitutions (preferably conservative or highly conservative amino acid substitutions) in 2 or less positions selected from positions 1 and 4 of SEQ ID NO: 12; (b) a CDR-H2 having the amino acid sequence of SEQ ID NO:7 or a variant thereof having amino acid substitutions (preferably conservative or highly conservative amino acid substitutions) in 3 or less positions selected from positions 5, 8 and 13 of SEQ ID NO: 7, and (c) a CDR-H3 having the amino acid sequence of SEQ ID NO: 14 or a variant thereof having amino acid substitutions (preferably conservative or highly conservative amino acid substitutions) in 2 or less positions selected from positions 3 and 7 of SEQ ID NO: 14; and
ii) a light chain variable domain (VL) comprising (d) a CDR-L1 having the amino acid sequence of SEQ ID NO: 15 or a variant thereof having amino acid substitutions (preferably conservative or highly conservative amino acid substitutions) in 2 or less positions selected from positions 8 and 10 of SEQ ID NO: 15 and (f) a CDR-L3 having the amino acid sequence of SEQ ID NO: 18 or a variant thereof having amino acid substitutions (preferably conservative or highly conservative amino acid substitutions) in 3 or less positions selected from positions 4, 5 and 6 of SEQ ID NO: 18.

In embodiments, the variant of SEQ ID NO: 12 has a M, L, I or V in position 4 and a N, S, T or Q in position 5; the variant of SEQ ID NO: 7 has a I, A, L or V in position 2; a T or S in position 4; a R, G, D or K in position 5; the variant of SEQ ID NO: 14 has a H, A, R or K in position 3 and a N, A or Q in position 7; the variant of SEQ ID NO: 15 has a N, Q, S or T in position 9 and A, N or V in position 10; and/or the variant of SEQ ID NO:18 has a G, A or S in position 6; a S, G, N or Q in position 7; T, S or G in position 8; and V, A, I or L in position 12.

In specific embodiments, the variant of SEQ ID NO: 12 has a M, L, or I in position 4 and a N, S or T in position 5; the variant of SEQ ID NO: 7 has a I, A or L in position 2; a T or S in position 4; a R, G, D or K in position 5; the variant of SEQ ID NO: 14 has a H, A, R or K in position 3 and a N, A or Q in position 7; the variant of SEQ ID NO: 15 has a N, Q, S or T in position 9 and A, N or V in position 10; and/or the variant of SEQ ID NO:18 has a G, A or S in position 6; a S, G or N in position 7; T, S or G in position 8; and V or A in position 12.

In even more specific embodiments, the variant of SEQ ID NO: 12 has a M or L in position 4 and a N in position 5; the variant of SEQ ID NO: 7 has a I in position 2; a T in position 4; a R, G or D in position 5; the variant of SEQ ID NO: 14 has a H or A in position 3 and a N or A in position 7; the variant of SEQ ID NO: 15 has a N in position 9 and A or N in position 10; and/or the variant of SEQ ID NO:18 has a G, A or S in position 6; a S, G or N in position 7; T, S or G in position 8; and/or V or A in position 12.

In a preferred embodiment, the variants of SEQ ID NOs: 12, 7, 14, 9 and 17 have compared to SEQ ID NOs: 12, 7, 14, 15 and 18, respectively, amino acid substitutions (preferably conservative or highly conservative amino acid substitutions) at 12 or less positions.

As demonstrated in the appended Examples the CDR-L2 region is of the T4 specific antibodies of the invention does not contribute to the interaction with T4. Accordingly, the CDR-L2 can, in principle, have any sequences. In preferred embodiments the light chain variable domain comprises (e) a CDR-L2 having the amino acid sequence of SEQ ID NO: 11 or a variant thereof having amino acid substitutions (preferably conservative or highly conservative amino acid substitutions) in 5 or less, 4 or less, 3 or less, 2 or less or 1 or less positions of SEQ ID NO: 11. In an embodiment, the variant has no amino acid substitution in positions 1 and 2 of SEQ ID NO: 11. In a particular embodiment, the light chain variable domain of the monoclonal antibody of the invention comprises (e) a CDR-L2 having the amino acid sequence of SEQ ID NO: 11 or a variant thereof having amino acid substitution (preferably conservative or highly conservative amino acid substitution) in 1 or less positions of SEQ ID NO: 11. For example, the light chain variable domain of the monoclonal antibody of the invention may comprise e) a CDR-L2 having the amino acid sequence of SEQ ID NO: 11 or a variant thereof having 1 or less amino acid substitution in position 6 of SEQ ID NO: 11.

In embodiments, any of the amino acid substitutions as mentioned herein may be a conservative amino acid substitution of the respective amino acid defined in the respective position of the sequence.

As used in the context of the invention, a “conservative amino acid substitution” means the substitution of an amino acid with another amino acid selected from its same physicochemical group, wherein the physicochemical groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu
   wherein if Cys is to be conservatively substituted, it is substituted with Ser or Ala, and
   wherein if Pro is to be conservatively substituted it is substituted with Ala.

In embodiments, any of the amino acid substitutions as mentioned herein may be a highly conservative amino acid substitution of the respective amino acid defined in the respective position of the sequence.

As used in the context of the invention, a “highly conservative amino acid substitution” means the following amino acid substitutions:
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

In embodiments, the heavy chain variable domain (VH) of the monoclonal antibody of the invention comprises (a) a CDR-H1 comprising or consisting of the amino acid sequence of X1NVX2N (wherein X1 is S or R and X2 is M or L; also referred to herein as SEQ ID NO: 19 or SEQ ID Nos: 19); (b) a CDR-H2 comprising or consisting of the amino acid sequence of SEQ ID NO: 20, and (c) a CDR-H3 comprising or consisting of the amino acid sequence of SEQ ID NO: 21; and the light chain variable domain (VL) of the antibody of the invention comprises (d) a CDR-L1 comprising or consisting of the amino acid sequence of SEQ ID NO: 22 and (f) a CDR-L3 comprising or consisting of the amino acid sequence of SEQ ID NO: 23.

Accordingly, in one aspect provided is a monoclonal antibody specifically binding to T4, wherein said monoclonal antibody comprises:
i) a heavy chain variable domain (VH) of the monoclonal antibody of the invention comprises (a) a CDR-H1 comprising or consisting of the amino acid sequence of X1NVX2N (wherein X1 is S or R and X2 is M or L); (b) a CDR-H2 comprising or consisting the amino acid sequence of SEQ ID NO: 20, and (c) a CDR-H3 comprising or consisting of the amino acid sequence of SEQ ID NO: 21; and
ii) a light chain variable domain (VL) of the antibody of the invention comprises (d) a CDR-L1 comprising or consisting of the amino acid sequence of SEQ ID NO: 22 and (f) a CDR-L3 comprising or consisting of the amino acid sequence of SEQ ID NO: 23.

The monoclonal antibody of the invention may further comprise a CDR-L2 in its VL comprising or consisting of the amino acid sequence of SEQ ID NO: 24.

Accordingly, herein provided is a monoclonal antibody specifically binding to T4, wherein said monoclonal antibody comprises:
i) a heavy chain variable domain (VH) of the monoclonal antibody of the invention comprises (a) a CDR-H1 comprising or consisting of the amino acid sequence of X1NVX2N (wherein X1 is S or R and X2 is M or L); (b) a CDR-H2 comprising or consisting the amino acid sequence of SEQ ID NO: 20, and (c) a CDR-H3 comprising or consisting of the amino acid sequence of SEQ ID NO: 21; and
ii) a light chain variable domain (VL) of the antibody of the invention comprises (d) a CDR-L1 comprising or consisting of the amino acid sequence of SEQ ID NO: 22; (e) a CDR-L2 comprising or consisting of the amino acid sequence of SEQ ID NO: 24; and (f) a CDR-L3 comprising or consisting of the amino acid sequence of SEQ ID NO: 23.

In embodiments, the heavy chain variable domain (VH) of the antibody of the invention comprises (a) a CDR-H1 comprising or consisting of an amino acid sequence selected from the group consisting of SEQ ID NOs: 12, 25 and 26 (b) a CDR-H2 comprising or consisting of an amino acid sequence selected from the group consisting of SEQ ID NOs: 7, 27 and 28, and (c) a CDR-H3 comprising or consisting of an amino acid sequence selected from the group consisting of SEQ ID NOs: 14 and 29; and the light chain variable domain (VL) of the antibody of the invention comprises (d) a CDR-L1 comprising or consisting of an amino acid sequence selected from the group consisting of SEQ ID NOs: 15 and 30 and (f) a CDR-L3 comprising or consisting of an amino acid sequence selected from the group consisting of SEQ ID NOs: 18, 31 and 32.

Accordingly, herein provided is a monoclonal antibody specifically binding to T4, wherein said monoclonal antibody comprises:
(i) a heavy chain variable domain (VH) comprising (a) a CDR-H1 comprising or consisting of an amino acid sequence selected from the group consisting of SEQ ID NOs: 12, 25 and 26 (b) a CDR-H2 comprising or consisting of an amino acid sequence selected from the group consisting of SEQ ID NOs: 7, 27 and 28, and (c) a CDR-H3 comprising or consisting of an amino acid sequence selected from the group consisting of SEQ ID NOs: 14 and 29; and
(ii) a light chain variable domain (VL) comprising (d) a CDR-L1 comprising or consisting of an amino acid sequence selected from the group consisting of SEQ ID NOs: 15 and 30 and (f) a CDR-L3 comprising or consisting of an amino acid sequence selected from the group consisting of SEQ ID NOs: 18, 31 and 32.

The light chain variable domain (VL) may further comprises a CDR-L2 comprising or consisting of the amino acid sequence of SEQ ID NO: 11 or 33.

Accordingly, in embodiments provided is a monoclonal antibody specifically binding to T4, wherein said monoclonal antibody comprises:
a heavy chain variable domain (VH) comprising (a) a CDR-H1 comprising or consisting of an amino acid sequence selected from the group consisting of SEQ ID NOs: 12, 25 and 26 (b) a CDR-H2 comprising or consisting of an amino acid sequence selected from the group consisting of SEQ ID NOs: 7, 27 and 28, and (c) a CDR-H3 comprising or consisting of an amino acid sequence selected from the group consisting of SEQ ID NOs: 14 and 29; and
a light chain variable domain (VL) comprising (d) a CDR-L1 comprising or consisting of an amino acid sequence selected from the group consisting of SEQ ID NOs: 15 and 30; € a CDR-L2 comprising or consisting of the amino acid sequence of SEQ ID NO: 11 or 33; and (f) a CDR-L3 comprising or consisting of an amino acid sequence selected from the group consisting of SEQ ID NOs: 18, 31 and 32.

The antibody of the invention may have different framework regions flanking the CDRs in the VH and VL domain.

The heavy chain variable domain (VH) may comprises framework regions (FW) flanking the CDRs of the VH as represented in formula I:

FW-H1-CDR-H1-FW-H2-CDR-H2-FW-H3-CDR-H3-FW-H4 (formula I).

The FW-H1 may have the amino acid sequence of SEQ ID NO: 34 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 34.

The FW-H2 may have the amino acid sequence of SEQ ID NO: 35 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 35.

The FW-H3 may have the amino acid sequence of SEQ ID NO: 36 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 36.

The FW-H4 may have the amino acid sequence of SEQ ID NO: 37 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 37.

The light chain variable domain (VL) may comprise framework regions (FW) flanking the CDRs of the VL as represented in formula I:

FW-L1-CDR-L1-FW-L2-CDR-L2-FW-L3-CDR-L3-FW-L4 (formula I).

The FW-L1 may have the amino acid sequence of SEQ ID NO: 38 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 38.

The FW-L2 may have the amino acid sequence of SEQ ID NO: 39 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 39.

The FW-L3 may have the amino acid sequence of SEQ ID NO: 40 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 40.

The FW-L4 may have the amino acid sequence of SEQ ID NO: 41 or a variant thereof having at least 60%, preferably at least 70%, even more preferably at least 80%, even more preferably at least 85%, even more preferably at least 90%, even more preferably at least 95%, even more preferably at least 99% sequence identity SEQ ID NO: 41.

In embodiments, the FW-H1 may have the amino acid sequence of SEQ ID NO: 34 or a variant thereof having at least 60% sequence identity, the FW-H2 may have the amino acid sequence of SEQ ID NO: 35 or a variant thereof having at least 60% sequence identity, the FW-H3 may have the amino acid sequence of SEQ ID NO: 36 or a variant thereof having at least 60% sequence identity and the FW-H4 may have the amino acid sequence of SEQ ID NO: 37 or a variant thereof having at least 60% sequence identity; and/or the FW-L1 may have the amino acid sequence of SEQ ID NO: 38 or a variant thereof having at least 60% sequence identity thereto, the FW-L2 may have the amino acid sequence of SEQ ID NO: 39 or a variant thereof having at least 60% sequence identity thereto, the FW-L3 may have the amino acid sequence of SEQ ID NO: 40 or a variant thereof having at least 60% sequence identity thereto and the FW-L4 may have the amino acid sequence of SEQ ID NO: 41 or a variant thereof having at least 60% sequence identity thereto.

In embodiments, the FW-H1 may have the amino acid sequence of SEQ ID NO: 34 or a variant thereof having at least 70% sequence identity, the FW-H2 may have the amino acid sequence of SEQ ID NO: 35 or a variant thereof having at least 70% sequence identity, the FW-H3 may have the amino acid sequence of SEQ ID NO: 36 or a variant thereof having at least 70% sequence identity and the FW-H4 may have the amino acid sequence of SEQ ID NO: 37 or a variant thereof having at least 70% sequence identity; and/or the FW-L1 may have the amino acid sequence of SEQ ID NO: 38 or a variant thereof having at least 70% sequence identity thereto, the FW-L2 may have the amino acid sequence of SEQ ID NO: 39 or a variant thereof having at least 70% sequence identity thereto, the FW-L3 may have the amino acid sequence of SEQ ID NO: 40 or a variant thereof having at least 70% sequence identity thereto and the FW-L4 may have the amino acid sequence of SEQ ID NO: 41 or a variant thereof having at least 70% sequence identity thereto.

In embodiments, the FW-H1 may have the amino acid sequence of SEQ ID NO: 34 or a variant thereof having at least 80% sequence identity, the FW-H2 may have the amino acid sequence of SEQ ID NO: 35 or a variant thereof having at least 80% sequence identity, the FW-H3 may have the amino acid sequence of SEQ ID NO: 36 or a variant thereof having at least 80% sequence identity and the FW-H4 may have the amino acid sequence of SEQ ID NO: 37 or a variant thereof having at least 80% sequence identity; and/or the FW-L1 may have the amino acid sequence of SEQ ID NO: 38 or a variant thereof having at least 80% sequence identity thereto, the FW-L2 may have the amino acid sequence of SEQ ID NO: 39 or a variant thereof having at least 80% sequence identity thereto, the FW-L3 may have the amino acid sequence of SEQ ID NO: 40 or a variant thereof having at least 80% sequence identity thereto and the FW-L4 may have the amino acid sequence of SEQ ID NO: 41 or a variant thereof having at least 80% sequence identity thereto.

In embodiments, the FW-H1 may have the amino acid sequence of SEQ ID NO: 34 or a variant thereof having at least 85% sequence identity, the FW-H2 may have the amino acid sequence of SEQ ID NO: 35 or a variant thereof having at least 85% sequence identity, the FW-H3 may have the amino acid sequence of SEQ ID NO: 36 or a variant thereof having at least 85% sequence identity and the FW-H4 may have the amino acid sequence of SEQ ID NO: 37 or a variant thereof having at least 85% sequence identity; and/or the FW-L1 may have the amino acid sequence of SEQ ID NO: 38 or a variant thereof having at least 85% sequence identity thereto, the FW-L2 may have the amino acid sequence of SEQ ID NO: 39 or a variant thereof having at least 85% sequence identity thereto, the FW-L3 may have the amino acid sequence of SEQ ID NO: 40 or a variant thereof having at least 85% sequence identity thereto and the FW-L4 may have the amino acid sequence of SEQ ID NO: 41 or a variant thereof having at least 85% sequence identity thereto.

In embodiments, the FW-H1 may have the amino acid sequence of SEQ ID NO: 34 or a variant thereof having at least 90% sequence identity, the FW-H2 may have the amino acid sequence of SEQ ID NO: 35 or a variant thereof having at least 90% sequence identity, the FW-H3 may have the amino acid sequence of SEQ ID NO: 36 or a variant thereof having at least 90% sequence identity and the FW-H4 may have the amino acid sequence of SEQ ID NO: 37 or a variant thereof having at least 90% sequence identity; and/or the FW-L1 may have the amino acid sequence of SEQ ID NO: 38 or a variant thereof having at least 90% sequence identity thereto, the FW-L2 may have the amino acid sequence of SEQ ID NO: 39 or a variant thereof having at least 90% sequence identity thereto, the FW-L3 may have the amino acid sequence of SEQ ID NO: 40 or a variant thereof having at least 90% sequence identity thereto and the FW-L4 may have the amino acid sequence of SEQ ID NO: 41 or a variant thereof having at least 90% sequence identity thereto.

In embodiments, the FW-H1 may have the amino acid sequence of SEQ ID NO: 34 or a variant thereof having at least 95% sequence identity, the FW-H2 may have the amino acid sequence of SEQ ID NO: 35 or a variant thereof having at least 95% sequence identity, the FW-H3 may have the amino acid sequence of SEQ ID NO: 36 or a variant thereof having at least 95% sequence identity and the FW-H4 may have the amino acid sequence of SEQ ID NO: 37 or a variant thereof having at least 95% sequence identity and/or the FW-L1 may have the amino acid sequence of SEQ ID NO: 38 or a variant thereof having at least 95% sequence identity thereto, the FW-L2 may have the amino acid sequence of SEQ ID NO: 39 or a variant thereof having at least 95% sequence identity thereto, the FW-L3 may have the amino acid sequence of SEQ ID NO: 40 or a variant thereof having at least 95% sequence identity thereto and the FW-L4 may have the amino acid sequence of SEQ ID NO: 41 or a variant thereof having at least 95% sequence identity thereto.

In embodiments, the FW-H1 may have the amino acid sequence of SEQ ID NO: 34 or a variant thereof having at least 99% sequence identity, the FW-H2 may have the amino acid sequence of SEQ ID NO: 35 or a variant thereof having at least 99% sequence identity, the FW-H3 may have the amino acid sequence of SEQ ID NO: 36 or a variant thereof having at least 99% sequence identity and the FW-H4 may have the amino acid sequence of SEQ ID NO: 37 or a variant thereof having at least 99% sequence identity; and/or the FW-L1 may have the amino acid sequence of SEQ ID NO: 38 or a variant thereof having at least 99% sequence identity thereto, the FW-L2 may have the amino acid sequence of SEQ ID NO: 39 or a variant thereof having at least 99% sequence identity thereto, the FW-L3 may have the amino acid sequence of SEQ ID NO: 40 or a variant thereof having at least 99% sequence identity thereto and the FW-L4 may have the amino acid sequence of SEQ ID NO: 41 or a variant thereof having at least 99% sequence identity thereto.

In embodiments, the FW-H1 may have the amino acid sequence of SEQ ID NO: 34, the FW-H2 may have the amino acid sequence of SEQ ID NO: 35, the FW-H3 may have the amino acid sequence of SEQ ID NO: 36 and the FW-H4 may have the amino acid sequence of SEQ ID NO: 37.

In embodiments, the VH of the monoclonal antibody of the invention comprises or consists of the amino acid sequence of SEQ ID NO: 42 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 42.

In other embodiments, the VH of the monoclonal antibody of the invention comprises or consists of the amino acid sequence of SEQ ID NO: 43 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 43.

In yet another embodiment, the VH of the monoclonal antibody of the invention comprises or consists of the amino acid sequence of SEQ ID NO: 44 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 44.

In embodiments, the VL of the monoclonal antibody of the invention comprises or consists of the amino acid sequence of SEQ ID NO: 45 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 45.

In other embodiments, the VL of the monoclonal antibody of the invention comprises or consists of the amino acid sequence of SEQ ID NO: 46 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 46.

In yet another embodiment, the VL of the monoclonal antibody of the invention comprises or consists of the amino acid sequence of SEQ ID NO: 47 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 47.

Provided herein is, for example, a monoclonal antibody specifically binding to T4, wherein said monoclonal antibody comprises a VH comprising or consisting of the amino acid sequence of SEQ ID NO: 42 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 42; and a VL comprising or consisting of the amino acid sequence of SEQ ID NO: 45 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 45.

Furthermore, provided herein is, for example, a monoclonal antibody specifically binding to T4, wherein said monoclonal antibody comprises a VH comprising or consisting of the amino acid sequence of SEQ ID NO: 43 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 43; and VL comprising or consisting of the amino acid sequence of SEQ ID NO: 46 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 46.

Furthermore, also provided herein is, for example, a monoclonal antibody specifically binding to T4, wherein said monoclonal antibody comprises a VH comprising or consisting of the amino acid sequence of SEQ ID NO: 44 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 44; and VL comprising or consisting of the amino acid sequence of SEQ ID NO: 47 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 47.

As demonstrated by the appended Examples, the monoclonal antibody of the invention is characterized by a particularly high association rate constant (kₐ) for the binding to free L-T4. This fast association rate makes the antibody of the invention particularly suitable for a high throughput immunoassay and a competitive assay format.

Accordingly, in embodiments the monoclonal antibody of the invention is further characterized in that the association rate constant (kₐ) for the binding to T4 as measured at 37° C. is at least 1.9*10⁷ M⁻¹s⁻¹. In embodiments, the association rate for the binding to T4 as measured at 37° C. is at least 2*10⁷ M⁻¹s⁻¹. In embodiments, the association rate constant (kₐ) for the binding to T4 as measured at 37° C. is at least 1*108 M⁻¹s⁻¹. In embodiments, the association rate for the binding to T4 as measured at 37° C. is at least 1*10⁹ M⁻¹s⁻¹.

In embodiments, the monoclonal antibody of the invention is further characterized in that the association rate constant (kₐ) for the binding to T4 corresponds to at least 10%, preferably at least 20%, even more preferably at least 30%, even more preferably at least 50%, even more preferably at least 70%, even more preferably at least 80% and even more preferably at least 90% of the association rate constant (kₐ) for the binding to T4 of the antibody comprising or consisting of a heavy chain of SEQ ID NO: 48 and a light chain of SEQ ID NO: 49, wherein the association rates are measured under the identical experimental conditions. The experimental conditions are preferably as described herein below and/or in Example 3.

In embodiments the monoclonal antibody of the invention is an antibody comprising a Fab fragment and is further characterized in that the association rate constant (kₐ) of said Fab fragment for the binding to T4 as measured at 37° C. is at least 1.9*10⁷ M⁻¹s⁻¹. In embodiments, the association rate constant (kₐ) of the Fab antibody fragment for the binding to T4 as measured at 37° C. is at least 2*10⁷ M⁻¹s⁻¹. In embodiments, the association rate constant (kₐ) of the Fab fragment for the binding to T4 as measured at 37° C. is at least 1*108 M⁻¹s⁻¹. In embodiments, the association rate constant (kₐ) of the Fab fragment for the binding to T4 as measured at 37° C. is at least 1*10⁹ M⁻¹s⁻¹.

In embodiments the monoclonal antibody of the invention is a Fab antibody fragment and is further characterized in that the association rate constant (kₐ) for the binding to T4 as measured at 37° C. is at least 1.9*10⁷ M⁻¹s⁻¹. In embodiments, the association rate constant (kₐ) of the Fab antibody fragment for the binding to T4 as measured at 37° C. is at least 2*10⁷ M⁻¹s⁻¹. In embodiments, the association rate constant (kₐ) of the Fab fragment for the binding to T4 as measured at 37° C. is at least 1*108 M⁻¹s⁻¹. In embodiments, the association rate constant (kₐ) of the Fab fragment for the binding to T4 as measured at 37° C. is at least 1*10⁹ M⁻¹s⁻¹.

Methods for determining that the association rate constant (kₐ) of an antibody for the binding to T4 are known in the art. Exemplary examples are described in the appended Examples (in particular Example 3).

In embodiments, the settings of the method for determining the association rate constant (kₐ) of an antibody for the binding to T4 may be calibrated such that for an Fab antibody fragment consisting of a heavy chain of SEQ ID NO: 48 and a light chain of SEQ ID NO: 49 the association rate constant for the binding to T4 as measured at 37° C. is 1*10⁹ M⁻¹s⁻¹.

In a preferred embodiment, the association rate constant (kₐ) of the antibody of the invention for the binding to T4 is measured by surface plasmon resonance spectroscopy (e.g. BIAcore®).

As described in the appended Examples, the antibodies 38F8, 7E10 and 7D4 showed significant mass transport limitation (MTL) in BIAcore® experiments. Accordingly, the surface plasmon resonance spectroscopy determination used for determining the association rate constant must use a MTL correction. The MTL correction is preferably based on a 2-compartment model (Myszka et. al., Biophysical Journal, Vol75, August 1998, 583-594; Biacore Insight Evaluation Software User Manual 29287248 AB, page 214-215). In a preferred embodiment, the analysis may be conducted with a GE Healthcare Biacore™ 8K instrument and the analysis may be conducted automatically with the Evaluation Insight Software V3.011.15423.

Surface plasmon resonance measurements for determining the kₐ may be conducted at a temperature of 37° C. The measurement may be conducted using multi cycle kinetics with a series of increasing L-T4 concentrations, e.g. c=0.12-10 nM, dilution factor 3. The system buffer may be PBS, pH 7.4 containing 11 mM PO₄, 137 mM NaCl, 2.7 mM KCl, pH 7.4+0.05% (w/v) Tween20 and 5% (v/v) DMSO. As sample buffer the latter system buffer supplemented with 1 mg/mL Carboxymethyldextran (CMD) may be used.

The association rate constant constant kₐ [1/Ms], the dissociation rate constant k_{d} [s⁻¹] and the dissociation equilibrium constant K_{D} [M] may be calculated according to a Langmuir model, e.g. by using the evaluation software corresponding to the instrument (e.g. as specified above).

As demonstrated in the appended examples, the antibody of the invention is also characterized by having a high affinity to L-T4. Accordingly, the monoclonal antibody of the invention may be characterized by having an equilibrium dissociation constant K_{D} for the binding to free L-T4 of 10 nM or less, preferably 5 nM or less, preferably 1 nM or less, even more preferably 0.5 nM or less even more preferably 0.3 nM or less and most preferably 0.28 nM or less. These values are preferably determined using Biacore surface plasmon resonance spectroscopy and an affinity in solution setting. The experimental conditions are preferably as described herein below and/or in Example 3.

In embodiments, the monoclonal antibody of the invention is characterized in that the K_{D} for the binding to T4 corresponds to at least 10%, preferably at least 20%, even more preferably at least 30%, even more preferably at least 50%, even more preferably at least 70%, even more preferably at least 80% and even more preferably at least 90% of the K_{D} for the binding to free T4 of the antibody comprising or consisting of a heavy chain of SEQ ID NO: 48 and a light chain of SEQ ID NO: 49, wherein the K_{D} values are measured under the identical experimental conditions. The experimental conditions are preferably as described herein below and/or in Example 3. The preferred temperature for determining the K_{D} is 37° C.

In embodiments the monoclonal antibody of the invention is an antibody comprising a Fab fragment and is further characterized in that equilibrium dissociation constant K_{D} for the binding to free L-T4 of 10 nM or less, preferably 5 nM or less, preferably 1 nM or less, even more preferably 0.5 nM or less and most preferably 0.3 nM or less. These values are preferably determined using Biacore surface plasmon resonance spectroscopy and an affinity in solution setting. The experimental conditions are preferably as described herein below and/or in Example 3. The preferred temperature for determining the K_{D} is 37° C.

In embodiments the monoclonal antibody of the invention is a Fab antibody fragment and is further characterized in that the equilibrium dissociation constant K_{D} for the binding to free L-T4 of 10 nM or less, preferably 5 nM or less, preferably 1 nM or less, even more preferably 0.5 nM or less and most preferably 0.3 nM or less. These values are preferably determined using Biacore surface plasmon resonance spectroscopy and an affinity in solution setting. The experimental conditions are preferably as described herein below and/or in Example 3. The preferred temperature for determining the K_{D} is 37° C.

Methods for determining an equilibrium dissociation constant K_{D} are known in the art.

In a preferred embodiment, the K_{D} may be determined by surface plasmon resonance spectroscopy (e.g. BIAcore®).

The preferred temperature for determining the K_{D} is 37° C.

In preferred embodiments, the method for determining the K_{D} is calibrated such that the K_{D} such that for an Fab antibody fragment consisting of a heavy chain of SEQ ID NO: 48 and a light chain of SEQ ID NO: 49 the K_{D} for the binding to T4 is 0.28 nM at 37° C.

In embodiments, the K_{D} may be determined using kinetic measurements determining the association and dissociation rate. Accordingly, the methods may in embodiments be the same surface plasmon resonance methods as described for determination of the kₐ, above. Said method preferably comprises correction for mass transport limitation.

In preferred embodiments, the K_{D} may be determined using surface plasmon resonance spectroscopy (e.g. BIAcore®) and using an affinity in solution measurement principle. The advantage of affinity in solution analysis is that it does not underlie mass transportation limitation and that it allows measurement in equilibrium.

For affinity in solution measurements of the K_{D}, free L-T4-conjugated to a capture label (e.g. biotin) may be pre-captured (e.g. reversibly) on a sensor surface (e.g. on a CAP-chip via Streptavidin (SA)-Biotin interaction if the capture label is biotin). Mixtures of the anti-T4 antibody or antigen-binding fragment thereof (e.g. Fab) and non-labeled L-T4 may be pre-incubated for several hours for reaching equilibrium. The concentration of the ‘free’ (i.e. not L-T4 bound) L-T4 antibody or the fragment thereof may then be determined via binding to the surface-displayed L-T4 using a preceding calibration for quantification. The antibody or antigen-binding fragment thereof concentration is preferably held constant in the mixtures, while the L-T4 concentration is varied. With increasing L-T4 present, the ‘free’ Fab fragment in solution decreases and the K_{D} can be determined.

An exemplary setup for affinity in solution determination of the K_{D} for the binding of an antibody of the invention to free L-T4 may be as follows: Following the vendor instructions for the CAP-Kit (Cytiva), subsequently to the CAP-Reagent the biotinylated T4 may be reversibly captured on the sensor surface with high density. Regeneration may be performed after each cycle using Guanidinium/NaOH solution. Preincubation of both interaction partners in solution may be conducted as follows: Antibody or antigen binding fragment (e.g. Fab) concentration may be kept constant at 3 nM. L-T4 Thyroid hormone concentration may be optimized for each T4 interaction. For instance, 0.021 nM-90 nM unlabeled L-T4 may be used for the pre-incubation.

The Affinity in Solution model from Biacore Evaluation software (e.g. Biacore T200 Evaluation SW V3.2) may be used to evaluate the data and to determine the K_{D}.

As used herein, the phrase “specifically binds” in the context of an antibody or antibody antigen binding fragment reacting with T4 indicates that the T4 is bound to the antibody or antibody antigen binding fragment via an antigen-antibody reaction.

In embodiments, the antibody of the invention discriminates free L-T4 from structurally related compounds such as 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine. As used herein “discriminate” or “discriminating” means that the binding affinity to L-T4 is considerably higher than for a related compound, i.e. the K_{D} is considerably lower. In a preferred embodiment, “discriminates” means that the antibody of the invention does show no detectable binding to the related compound or the ratio of the K_{D} for the related compound (K_{D}-XR) and the K_{D} for L-T4 as measured at 37° C. has a value of 4 or higher, preferably 5 or higher. In other words, the affinity to L-T4 compared to the affinity to the related compound is at least 4, preferably at least 5 times higher.

In embodiments, the antibody of the invention discriminates L-T4 from 3-iodo-L-Thyrosine (L-T3). Preferably, the ratio of the K_{D} for L-T3 and the K_{D} for L-T4 as measured at 25° C. may have a value of 4 or higher, preferably 20 or higher, even more preferably 30 or higher, even more preferably 40 or higher and most preferably 47 or higher. Alternatively or additionally, the ratio of the K_{D} for L-T3 and the K_{D} for L-T4 as measured at 37° C. may have a value of 4 or higher, preferably 20 or higher, even more preferably 30 or higher, even more preferably 40 or higher, even more preferably 50 or higher and most preferably 58 or higher.

Alternatively or additionally, the antibody of the invention discriminates L-T4 from rT3. Preferably, the ratio of the K_{D} for rT3 and the K_{D} for L-T4 as measured at 25° C. may have a value of 4 or higher, preferably 6 or higher, even more preferably 8 or higher and most preferably 10 or higher. Alternatively or additionally, the ratio of the K_{D} for rT3 and the K_{D} for L-T4 as measured at 37° C. may have a value of 4 or higher, preferably 8 or higher, even more preferably 10 or higher, even more preferably 12 or higher, even more preferably 14 or higher and most preferably 18 or higher.

Alternatively or additionally, the antibody of the invention discriminates L-T4 from 3,3′,5-tri-iodo-thyroacetic acid. Preferably, the ratio of the K_{D} for 3,3′,5-tri-iodo-thyroacetic acid and the K_{D} for L-T4 as measured at 37° C. has a value of 4 or higher, preferably 10 or higher, even more preferably 30 or higher, even more preferably 50 or higher, even more preferably 80 or higher and most preferably 92 or higher.

Alternatively or additionally, the antibody of the invention discriminates L-T4 from 3,3′,5,5′-tetra-iodothyroacetic acid. Preferably, the ratio of the K_{D} for 3,3′,5,5′-tetra-iodothyroacetic acid and the K_{D} for L-T4 as measured at 37° C. has a value of 3 or higher, preferably 4 or higher and most preferably 5 or higher.

Alternatively or additionally, the antibody of the invention discriminates L-T4 from 3,5-di-iodo-L-Thyrosine. Preferably, the ratio of the K_{D} for 3,5-di-iodo-L-Thyrosine and the K_{D} for L-T4 as measured at 37° C. has a value of 4 or higher, preferably 10 or higher, even more preferably 30 or higher, even more preferably 50 or higher, even more preferably 80, even ore preferably 100 or higher, even more preferably 150 or higher and most preferably 166 or higher.

Alternatively or additionally, the antibody of the invention discriminates L-T4 from 3-i-L-Thyrosine. Preferably, the ratio of the K_{D} for 3,5-di-iodo-L-Thyrosine and the K_{D} for L-T4 as measured at 37° C. has a value of 4 or higher. Even more preferably, the antibody of the invention does not show a interaction to 3-i-L-Thyrosine as measurable by surface plasmon resonance spectroscopy (e.g. using the settings as described herein, e.g. in Example 3).

Methods for determining the K_{D} for the binding of the antibody of the invention to L-T4 and any of the related compounds are state of the art. Exemplary methods for determining the K_{D} for the binding to L-T4 are described herein above and in the appended Examples. These methods can be applied mutatis mutandis to the related compounds such that ratios of the K_{D} values can be calculated. The “ratio of the K_{D} for a related compound and the K_{D} for L-T4” means that the K_{D} of the antibody for a related compound is divided by the K_{D} of the antibody for L-T4.

In preferred embodiments, the K_{D} values for calculating the ratios are determined with surface plasmon resonance spectroscopy (e.g. BIAcore®). In preferred embodiments, multi cycle kinetic measurements using potential cross reactant concentrations from 0.1 nM to 900 nM may be employed. The compounds structurally related to L-T4 may be injected using flow rates between 30 pL/min to 60 pL/min. The association phases may be monitored between 3 min to 5 min, the dissociation phases between 5 min to 15 minutes. The L-T4 interactions may be characterized by using an additional analyte injection with 30 min dissociation time. Surface plasmon resonance measurements for determining the K_{D} values may be conducted at a temperature of 37° C. The system buffer may be PBS, pH 7.4 containing 11 mM PO₄, 137 mM NaCl, 2.7 mM KCl, pH 7.4+0.05% (w/v) Tween20 and 5% (v/v) DMSO. As sample buffer the latter system buffer supplemented with 1 mg/mL Carboxymethyldextran (CMD) may be used. The dissociation equilibrium constant K_{D} [M] may be calculated according to a Langmuir model, e.g. by using the evaluation software corresponding to the instrument (e.g. as specified above).

As demonstrated in the appended Examples, the structurally related antibodies 38F8 7E10 and 7D4 showed a good signal to noise ratio in a competitive immunoassay. The performance was comparable to a polyclonal anti-L-T4 antibody.

Accordingly, in preferred embodiments, the T4 specific antibody of the invention when used in a competitive immunoassay for quantifying T4 (e.g. fT4) shows a signal-to-noise ratio that is at least 29%, at least 30%, at least 65%, at least 66%, at least 70%, at least 80%, at least 90%, or at least 95% of the signal-to noise ratio achieved with a Fab fragment comprising or consisting of the heavy chain sequence of SEQ ID NO: 48 and the light chain sequence of SEQ ID NO: 49. In these embodiments, the immunoassay setup is identical with the only exception that the antibody is exchanged.

In preferred embodiments, the signal-to-noise ratio may be analyzed in a fT4 concentration range of 0 to 122.1 pmol/L.

“Signal-to-noise ratio” as used herein may also be referred to as “signal dynamics ratio” or “relative total signal span”. For immunoassay formats in which the measured signal decreases with the increased presence of T4 (e.g. fT4) in the sample, the signal-to-noise ratio or signal dynamics ratio relates to the ratio calculated by dividing (i) the change in the signal (e.g. ECL counts) between a sample without T4 and a sample with the highest measured T4 (e.g. fT4) concentration (e.g. 122.1 pmol/L) by (ii) the signal (e.g. ECL counts) of the sample with the highest measured T4 concentration (e.g. 122.1 pmol/L). For immunoassay formats in which the measured signal increases with the increased presence of T4 (e.g. fT4) in the sample, the signal-to-noise ratio or signal dynamics ratio relates to the ratio calculated by dividing (i) the positive change in the signal (e.g. ECL counts) between a sample without T4 (e.g. fT4) and a sample with the highest measured T4 (e.g. fT4) concentration (e.g. 122.1 pmol/L)—or a defined analyte concentration in a medically relevant concentration range by (ii) the signal (e.g. ECL counts) of a sample without T4 (e.g. fT4).

The competitive immunoassay for assessing the signal to noise ratio of an antibody may have the following underlying assay principle:
(i) Incubating a sample comprising T4 (e.g. fT4) with the antibody, wherein the antibody is labelled with a detection label (e.g. a Ruthenium label) for a defined time (e.g. 9 min);
(ii) Adding to the mixture of (i) T4 coupled to a capture label (e.g. biotinylated T4 or T4(OSu)-bis-DADOO-Biotin-Hapten) and particles capable of binding to the capture label and incubating the resulting mixture for a defined time (e.g. 9 min); alternatively, the particles capable of binding to the capture label could be added in a subsequent step with incubating the resulting mixture for a defined time (e.g. 9 min).
(iii) Separating the particles from the mixture and detecting the labelled antibody bound thereto by measuring the detection label signal; and
(iv) Determining the amount of T4 (e.g. FT4) in the sample based on the measured detection label signal.

In this competitive assay principle, the higher the amount of T4 (e.g. FT4), the lower is the measured signal of the detection label.

A specific and particularly preferred example for a competitive immunoassay to assess the signal-to-noise ratio is provided in appended Example 4. Thus in specific embodiments, the signal-to-noise ratio may be assessed by a competitive immunological assay using an automated Elecsys® Immuno-Analyzer (e.g. Elecsys® Cobas® e411). The assay involving a total incubation time of 18 minutes may have the following settings: 1st incubation (9 min): 15 μL of a T4 (e.g. fT4) containing sample, 75 μL of ruthenylated L-T4-specific antibody are incubated; 2nd incubation (9 min): 75 μL of a solution of T4(OSu)-bis-DADOO-Biotin-Hapten-conjugate and 35 μL of Streptavidin-coated microparticles are added to the mixture of the first incubation step. Subsequently, the amount of T4 (e.g. fT4) in the sample may be determined by measuring the ruthenium label signal attached to the magnetic beads. Specifically, the reaction mixture may be aspirated into the measuring cell where the microparticles are magnetically captured onto the surface of the electrode. Unbound substances may then be removed with ProCell #11662988122 (Roche Diagnostics GmbH Germany). Application of a voltage to the electrode may then be used to induce electro-chemiluminescence-based emission of light, which is measured by a photomultiplier.

In embodiments, the Elecsys® Immuno-Analyzer based competitive assay as described above or in Example 4 below may be used. In these embodiments, for the antibody of the invention, the ratio of the detected counts for a first sample and a second sample (i.e. counts first sample divided by counts second sample) may be 11% or lower, 5% or lower or 4% or lower, given that the first sample has a T4 (e.g. fT4) concentration of 122.10 pmol/L and the second sample has a T4 (e.g. fT4) concentration of 0 pmol/L.

The antibodies and antigen binding fragments of the invention may be prepared by a variety of techniques routinely used in the art. For example, antibodies can be prepared immunizing a non-human animal (e.g. rabbits) with L-T4 isolating and subsequently isolating antigen-reactive, antibody producing B-cells. To enhance the immunogenicity, the L-T4 for immunization may be coupled to a carrier protein (e.g. keyhole limpet hemocyanin (KLH)). Preferably, T4-NH-PEG(3)-CO-KLH is used for immunization. Screening of antibodies binding to L-T4 may be achieved using L-T4 as analyte. Preferably, T4(OSu)-bis-DADOO-Biotin may be coupled to a surface and binding of antibodies thereto may be tested. To identify antibodies specifically binding to L-T4, a counter screening for binding to structurally related compounds such as 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine may be conducted. Preferred exemplary methods for the production of antibodies according to the invention using immunization of non-human animals are provided in the appended Examples (see in particular Example 1). Selected clones for producing antibodies can be processed according to routine methods for subsequent recombinant processing.

Another suitable method for producing or isolating antibodies and antibody antigen binding fragments of the invention include, but are not limited to, methods that select a recombinant antibody from a peptide or protein library (e.g., but not limited to, a bacteriophage, ribosome, oligonucleotide, RNA, cDNA, or yeast display library) using binding activities of interest. For example, antibodies or antigen binding fragments can be selected from such libraries by positively selecting for specific binding to L-T4, e.g. by using L-T4 coupled biotin (e.g. T4(OSu)-bis-DADOO-Biotin). Optionally, also a negative selection for binding to structurally related compounds, such as 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine, may be performed to identify antibodies specifically binding to L-T4. Display libraries are well known in the art and are, for example, available from various commercial vendors including but not limited to Cambridge Antibody Technologies (Cambridgeshire, UK), MorphoSys (Martinsried/Planegg, Del.), Biovation (Aberdeen, Scotland, UK) and Bioinvent (Lund, Sweden). Again, selected clones can be processed according to routine methods for subsequent recombinant processing.

Antibodies of the invention may be recombinantly expressed. Accordingly, in certain embodiments, the monoclonal antibody of the invention may be a recombinant antibody. Methods for producing a recombinant antibody are known in the art. An exemplary embodiment for the recombinant expression and subsequent purification of antibodies according to the present invention is provided in the appended Example 4.

In a second aspect, the present invention also provides a nucleic acid molecule encoding the monoclonal antibody of the invention or any antigen-binding fragments thereof, as defined herein above. In particular provided is a polynucleotide encoding a heavy chain and/or light chain variable domain of the monoclonal antibody specifically binding to T4 as defined herein above. In some embodiments, the polynucleotide may comprise further sequences to ensure that not only the heavy and/or light chain variable domain are expressed, but also the remaining heavy and/or light chain constant regions such that a full-length IgG antibody is expressed comprising the heavy and light chain variable domains of the invention. Accordingly, for each of the aspects and embodiments relating to monoclonal antibodies or antigen binding fragments specifically binding T4 as described herein a corresponding polynucleotide encoding the respective antibody or antigen binding fragment is provided herein.

In a third aspect, provided herein is a vector that comprises a polynucleotide of the invention. In particular, provided are vectors comprising a nucleic acid molecule encoding an antibody or antibody antigen binding fragment of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding the full length anti-L-T4/free L-T4 antibody antigen binding fragment as disclosed herein. As such, for each of the aspects and embodiments relating to monoclonal antibodies or antigen binding fragments specifically binding T4 as described herein a vector comprising the corresponding polynucleotide encoding the respective antibody or antigen binding fragment is provided herein.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, transactivators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains of the monoclonal antibodies specifically binding to T4 as disclosed herein. In some embodiments, the vector may comprise further sequences to ensure that not only the heavy and light chain variable domains are expressed, but also the remaining heavy and light chain constant regions such that a full-length IgG antibody is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the monoclonal antibody according to the invention) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population. Additionally, baculoviral systems can also be used as vector in eukaryotic expression system for the nucleic acid molecules of the invention.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis. The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells.

Accordingly, in a fourth aspect the present invention relates to a host cell comprising a polynucleotide according to the invention, or a vector according to the invention. The host cell may be a prokaryotic cell or a eukaryotic cell. In a preferred embodiment, the host cell is a eukaryotic cell. In a particular embodiment, the cell is a HEK cell. In another particular embodiment the host cell is a CHO cell.

When recombinant expression vectors encoding the heavy and/or light chain of the antibody of the invention as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Methods for purification of antibodies are well known in the art. Exemplary purification methods are described in the appended Examples

Accordingly, the invention also provides a method for the production of a monoclonal antibody specifically binding to T4 as disclosed herein. The method comprises culturing a host cell of the invention under suitable conditions and isolating the antibody produced. By purification steps, e.g. as described in the appended Examples an isolated monoclonal antibody of the invention can be derived. The invention further provides an antibody or an antigen binding fragment obtainable by any of the methods disclosed herein.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His6 tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

It will be appreciated that variations on the above procedures are within the scope of the present invention. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to remove parts of the encoding sequence(s) that are not necessary for maintaining specific and selective binding to the antigen(s) of interest. The molecules expressed from such truncated DNA molecules are also encompassed by the antibodies of the invention. Additionally, also provided are multivalent antibodies comprising a heavy and/or a light chain variable domain of the invention (e.g. forming and antibody Fv domain that specifically and selectively binds T4) at least twice (preferably four, five, six, seven or eight times).

Antibody derivatives can be produced, for example, by adding exogenous sequences to modify immunogenicity or reduce, enhance or modify binding, affinity, on-rate, off-rate, avidity, specificity, half-life, or any other suitable characteristic.

Also provided are humanized versions of the antibodies disclosed herein, i.e. comprising the CDRs of the heavy and or light chains as disclosed herein above. As well known in the art, “humanization” (to produce a humanized version of a parent antibody) refers to recombinantly engineering an antibody using CDRs derived from a non-human donor immunoglobulin in the context of human derived framework and constant domains. During the engineering, framework and/or CDR residues may be altered to preserve binding affinity and activity, e.g. specificity for T4. Methods to humanize antibodies are well known in the art, e.g. as disclosed in Queen et al., Proc. Natl. Acad Sci USA 86(1989), 10029-10032; Hodgson et al., Bio/Technology 9(1991) 421.

In a fifth aspect, the present invention provided a composition comprising an antibody of the invention, a polynucleotide of the invention, a vector of the invention, or a host cell of the invention. In a preferred embodiment, the composition is a diagnostic composition, i.e. a composition for use in diagnostic applications. In preferred embodiments, the composition is for use in an in vitro diagnostic test for detecting L-T4, preferably free L-T4. In a preferred embodiment the diagnostic composition may be a reagent for an immunoassay for detecting L-T4, preferably free L-T4. The diagnostic composition is preferably configured such that it allows for detection of L-T4, preferably free L-T4 in a sample obtained from a subject. The sample is preferably a blood sample (e.g. whole blood, serum or plasma).

In embodiments, the composition of the invention is a composition for in vitro detection (preferably quantification) of T4 (preferably fT4) in a sample, preferably using an immunoassay. In embodiments the immunoassay is a heterogeneous immunoassay. In embodiments, the immunoassay is a competitive immunoassay.

In a sixth aspect, the present invention provides for the use of an antibody according to the present invention for in vitro detection (preferably quantification) of T4 (e.g. free T4) in a sample The detection and/or quantification of T4 (e.g. free T4) is preferably achieved by using an immunoassay. Accordingly, also provided is an antibody of the invention for use in an immunoassay (e.g. a heterogeneous immunoassay) for detecting T4 (e.g. free T4).

The sample may be a body fluid, such as, but not restricted to, a blood sample, cerebrospinal fluid, seminal fluid, saliva or urine. In embodiments, the sample is a blood sample, such as whole blood, serum or plasma. In embodiments, the sample is serum or plasma.

The immunoassay is preferably competitive, as T4 is a very small analyte. In embodiments, the assay set up is a back titration type of competitive assay, e.g. as described herein below or in the appended Examples. In brief, the assay may be configured as follows:
(i) Incubating a sample comprising T4 (e.g. free T4) with the antibody, wherein the antibody is labelled with a detection label (e.g. a Ruthenium label) for a defined time (e.g. 9 min);
(ii) Adding to the mixture of (i) T4 coupled to a capture label (e.g. T4(OSu)-bis-DADOO-Biotin-Hapten) and particles capable of binding to the capture label and incubating the resulting mixture for a defined time (e.g. 9 min);
(iii) Separating the particles from the mixture and detecting the labelled antibody bound thereto by measuring the detection label signal; and
(iv) Determining the amount of T4 (e.g. free T4) based on the measured detection label signal.

Optionally, if total T4 is to be determined in a sample in which T4 is partially bound by binding proteins, step (i) may comprise releasing T4 from its binding proteins, as described in detail below in the context of the seventh aspect of the invention.

In a seventh aspect, the present invention provides for an in vitro immunoassay method for quantifying T4 (e.g. free T4) in a sample using the antibody of the invention. The antibody may be part of a composition, in particular a diagnostic composition of the invention.

The sample may be a body fluid, such as a blood sample, cerebrospinal fluid, seminal fluid, saliva or urine. In embodiments, the sample is a blood sample, such as whole blood, serum or plasma. In embodiments, the sample is serum or plasma.

The method for quantifying L-T4 may comprise (i) incubating a sample comprising L-T4 with the antibody of the invention under conditions allowing the antibody to bind L-T4 and (ii) quantifying the amount of L-T4 in the sample by directly or indirectly detecting the amount of L-T4 bound to the antibody of the invention.

Directly detecting means that the amount of antibody-L-T4 complexes are detected directly via a detection label and the amount of L-T4-antibody complexes proportionally corresponds to the amount of L-T4 in the sample. Indirectly detecting means that the amount of L-T4 indirectly correlates with the measured signal of the detectable label (e.g. as achieved in competitive assay formats). For example, for an indirect detection the antibody of the invention may be labeled with a detection label and the amount of binding of the labeled antibody to an analogon of L-T4 (i.e. competing with T4 for binding to the T4 antibody of the invention) or L-T4 linked to a surface (directly or via a capture label such as biotin) is detected. Thus, in embodiments, the amount of the antibody not binding to the L-T4 (e.g. fT4) in the sample is quantified and thereby the amount of L-T4 in the sample is indirectly determined.

In embodiments, the method of the invention may be a method for detecting total T4 (i.e. free T4 and T4 bound to binding proteins) in a sample. In these embodiments, the method may further comprise releasing T4 from binding proteins by adding a releasing agent. Such releasing agent may e.g. be added prior to or during incubation with the antibody. In embodiments, the releasing agent may be added prior to incubation with the antibody and may still be present during the further methods steps. Suitable reagents for releasing T4 from binding proteins are known in the art (e.g. see Method Sheet of Elecsys® T4; Ref. 09007784190). In embodiments, the releasing agent may be 8-anilino-1-naphthalene sulfonic acid (ANS). In embodiments ANS may be added at a final concentration in the sample between 0.3 mg/ml and 0.9 mg/ml, in embodiments 0.375 mg/ml to 0.83 mg/ml. In embodiments, ANS may be 0.83 mg/ml during the incubation with the antibody of the invention and may be 0.375 mg/ml in subsequent steps in which a capture agent (e.g. competing for the antibody of the invention is added.

The detection label linked to the antibody of the invention may be any detection label known in the art. For example, the detection label may be selected from an enzyme or a label emitting light (e.g. fluorescence, luminescence, chemiluminescence, electrochemiluminescence or radioactivity).

In an eighth aspect provided herein is a kit comprising the antibody of the invention or a composition comprising the same. In embodiments, the kit is a kit for detecting and/or quantifying T4 (e.g. fT4) in vitro. In embodiments, the kit is an immunoassay kit. In embodiments, the kit is a kit for an heterogenous immunoassay. In embodiments, the kit is a kit for a competitive immunoassay. In embodiments, the kit may be a kit for detecting total T4 in vitro and may further comprise a release agent for releasing T4 from binding proteins bound thereto in the sample (e.g. serum/plasma binding proteins.

The invention also provides a kit comprising any manufacture (e.g. a package or container or package insert) comprising at least one reagent of the present invention, i.e. one or more of (i) an antibody or antibody antigen binding fragment of the invention, (ii) a nucleic acid molecule of the invention, (iii) a vector of the invention, (iv) a host cell of the invention, and/or (v) an antibody or antibody antigen binding fragment produced or obtained by a method of the invention. The kit may be promoted, distributed, or sold as a unit for performing the methods of the present invention.

In embodiments, the kit of the invention may comprise an antibody of the invention that has a detection label (e.g. as specified herein elsewhere) attached thereto. The kit may further comprise L-T4. The L-T4 may comprise a capture label. The kit may comprise a solid phase such as magnetic particles (e.g. magnetic particles). The solid phase may be functionalized such that a capture label can bind thereto, e.g. may be functionalized with streptavidin. Alternatively, the solid phase may be pre-coated with L-T4.

As certain detection labels require substrates or reagents for generating a detectable signal, the kit of the invention may in embodiments additionally contain substrates and/or reagents allowing the detection of the detection label.

In a specific embodiment, the kit may comprise in a first container a monoclonal antibody specifically binding T4 (e.g. free T4) according to the present invention and in a separate second container a mixture of a solid phase and a capture analogue of L-T4 (e.g. biotinylated L-T4), wherein the capture analogue can bind to the solid phase. For example, the solid phase may be coated with streptavidin and the capture analogue may be biotinylated. Alternatively, the second container may comprise a solid phase immobilized with L-T4. In both alternatives the coupling of the L-T4 to the solid phase is such that the L-T4 can still be recognized by the antibody of the invention.

The present invention in particular also relates to the following items:
1. A monoclonal antibody specifically binding to L-thyroxine (T4), wherein said monoclonal antibody comprises:
   i) a heavy chain variable domain (VH) comprising V or A in position 33; Y in position 50; W in position 52; I in position 98, G, A or V in position 99; Y in position 100; and I in position 100b; and
   ii) a light chain variable domain (VL) comprising amino acids H or Y in position 28; N or K in position 29; W in position 32; G or A in position 91; Y, W or F in position 92; S or T in position 93; Y or F in position 95b; N, S, T or Q in position 95c; and H in position 96,
   wherein the positions of the amino acids in the VH and the VL are indicated according to the Kabat numbering scheme, respectively.
2. The monoclonal antibody of item 1, wherein the VL comprises N, S or T in position 95c.
3. The monoclonal antibody of item 1, wherein
   i) the VH comprises V in position 33 and G in position 99; and
   ii) the VL comprises G in position 91; Y or W in position 92; S in position 93; Y in position 95b and N or S in position 95c.
4. The monoclonal antibody of item 1, wherein
   i) the VH comprises V in position 33 and G in position 99; and
   ii) the VL comprises H in position 28; N in position 29; G in position 91; Y in position 92; S in position 93; Y in position 95b and N in position 95c.
5. The monoclonal antibody of any one of items 1 to 5, wherein the paratope of the monoclonal antibody for binding to T4 comprises the amino acids of the VH in positions 33, 50, 52, 98, 99, 100 and 100b and the amino acids of the VL in positions 28, 29, 32, 91, 92, 93, 95b, 95c and 96, wherein the positions of the amino acids in the VH and the VL are indicated according to the Kabat numbering scheme, respectively.
6. The monoclonal antibody of any one of items 1 to 5, wherein the VH comprises M, L, I or V in position 34; N, S, T or Q in position 35; I, A, L or V in position 51, T or S in position 52a; R, G, D or K in position 53; H, A, R or K in position 97; and/or N, A or Q in position 100a, wherein the positions of the amino acids are indicated according to the Kabat numbering scheme.
7. The monoclonal antibody of item 6, wherein the VH comprises M, L, or I in position 34; N, S or T in position 35; and/or I, A or L in position 51.
8. The monoclonal antibody of item 6, wherein the VH comprises M or L in position 34; N in position 35; I in position 51, T in position 52a; R, G or D in position 53; H or A in position 97; and/or N or A in position 100a.
9. The monoclonal antibody of item 6, wherein the VH comprises M in position 34; N in position 35; I in position 51, T in position 52a; R in position 53; H in position 97; and/or N in position 100a.
10. The monoclonal antibody of any one of items 1 to 9, wherein the VL comprises N, Q, S or T in position 30; A, N or V in position 31; G, A or S in position 94; S, G, N, Q in position 95; T, S or G in position 95a; and/or V, A, I or L in position 97, wherein the positions of the amino acids are indicated according to the Kabat numbering scheme.
11. The monoclonal antibody of item 10, wherein the VL comprises N in position 30; A or N in position 31; S, G or N in position 95; and/or V or A in position 97.
12. The monoclonal antibody of item 10, wherein the VL comprises N in position 30; A in position 31; G in position 94; S in position 95; T in position 95a and/or V in position 97.
13. The monoclonal antibody of any one of items 1 to 12, wherein the VH comprises amino acids other than proline in positions 31 and 32 according to the Kabat numbering scheme, respectively.
14. The monoclonal antibody of any one of item 1 to 12, wherein the VH comprises S, R or a conservative substitution thereof in position 31 according to the Kabat numbering scheme; and/or N or a conservative substitution thereof in position 32 according to the Kabat numbering scheme.
15. The monoclonal antibody of any one of item 1 to 12, wherein the VH comprises S or R in position 31 according to the Kabat numbering scheme; and/or N in position 32 according to the Kabat numbering scheme.
16. The monoclonal antibody of any one of items 1 to 15, wherein the CDR-H1 of VH comprises or consists of positions 31 to 35 according to the Kabat numbering scheme.
17. The monoclonal antibody of any one of items 1 to 16, wherein the VH comprises amino acids other than proline in positions 54 to 65 according to the Kabat numbering scheme.
18. The monoclonal antibody of any one of items 1 to 17, wherein the VH comprises the amino acid sequence SGNTYYASWAKG (SEQ ID NO: 1) in positions 54 to 65 according to the Kabat numbering scheme or a variant thereof having 12 or less, 11 or less, 10 or less, 9 or less, 8 or less, 7 or less, 6 or less, 5 or less, 4 or less, 3 or less, 2 or less, or 1 or less amino acid substitutions, wherein none of the positions 54 to 65 is proline.
19. The monoclonal antibody of item 18, wherein the 12 or less, 11 or less, 10 or less, 9 or less, 8 or less, 7 or less, 6 or less, 5 or less, 4 or less, 3 or less, 2 or less, or 1 or less amino acid substitutions are each individually conservative amino acid substitutions.
20. The monoclonal antibody of any one of items 1 to 19, wherein the VH comprises the amino acid sequence in positions 54 to 65 according to the Kabat numbering scheme selected from the group consisting of: SGNTYYASWAKG (SEQ ID NO: 1), SGNTYYATWAKG (SEQ ID NO: 2 or SGSTYYATWAKG (SEQ ID NO:3).
21. The monoclonal antibody of any one of items 1 to 20, wherein the CDR-H2 of VH comprises or consists of positions 50, 51, 52, 52a and 53 to 65 according to the Kabat numbering scheme.
22. The monoclonal antibody of any one of items 1 to 21, wherein the VH comprises amino acids other than proline in positions 95, 96, 100c, 101 and 102 according to the Kabat numbering scheme.
23. The monoclonal antibody of any one of items 1 to 22, wherein the VH comprises G or a conservative substitution thereof in position 95; L or a conservative substitution thereof in position 96; F or a conservative substitution thereof in position 100c; N or a conservative substitution thereof in position 101; and F or a conservative substitution thereof in position 102, wherein the positions of the amino acids are indicated according to the Kabat numbering scheme.
24. The monoclonal antibody of any one of items 1 to 23, wherein the VH comprises G in position 95; L in position 96; F in position 100c; N in position 101; and F in position 102, wherein the positions of the amino acids are indicated according to the Kabat numbering scheme.
25. The monoclonal antibody of any one of items 1 to 24, wherein the CDR-H3 of VH comprises or consists of positions 95 to 100, 100a, 100b, 100c, 101 and 102 according to the Kabat numbering scheme.
26. The monoclonal antibody of any one of items 1 to 25, wherein the VL comprises amino acids other than proline in positions 24, 25, 26, 27, 27a, 27b, 33 and 34 according to the Kabat numbering scheme.
27. The monoclonal antibody of any one of items 1 to 26, wherein the VL comprises Q or a conservative substitution thereof in position 24; S or a conservative substitution thereof in position 25; S or a conservative substitution thereof in position 26; Q or a conservative substitution thereof in position 27; S or a conservative substitution thereof in position 27a; V or a conservative substitution thereof in position 27b; C, L or a conservative substitution thereof in position 33; and/or S or a conservative substitution thereof in position 34, wherein the positions of the amino acids are indicated according to the Kabat numbering scheme.
28. The monoclonal antibody of any one of items 1 to 27, wherein the VL comprises Q in position 24; S in position 25; S in position 26; Q in position 27; S in position 27a; V in position 27b; C or L in position 33; and/or S in position 34, wherein the positions of the amino acids are indicated according to the Kabat numbering scheme.
29. The monoclonal antibody of any one of items 1 to 28, wherein the CDR-L1 of VL comprises or consists of positions 24, 25, 26, 27, 27a, 27b, 28, 29, 30, 31, 32, 33 and 34 according to the Kabat numbering scheme.
30. The monoclonal antibody of any one of items 1 to 29, wherein the VL comprises amino acids other than proline in positions 50, 51, 52, 53, 54, 55 and 56 according to the Kabat numbering scheme.
31. The monoclonal antibody of any one of items 1 to 30, wherein the VL comprises G or a conservative substitution thereof in position 50; A or a conservative substitution thereof in position 51; S or a conservative substitution thereof in position 52; T or a conservative substitution thereof in position 53; L or a conservative substitution thereof in position 54; T, A or a conservative substitution thereof in position 55; and/or C, S or a conservative substitution thereof in position 56, wherein the positions of the amino acids are indicated according to the Kabat numbering scheme.
32. The monoclonal antibody of any one of items 1 to 31, wherein the VL comprises GASTLTS (SEQ ID NO: 4) or GASTLAS (SEQ ID NO: 5) in positions 50 to 56 according to the Kabat numbering scheme.
33. The monoclonal antibody of any one of items 1 to 32, wherein the CDR-L2 of VL comprises or consists of positions 50, 51, 52, 53, 54, 55 and 56 according to the Kabat numbering scheme.
34. The monoclonal antibody of any one of items 1 to 33, wherein the VL comprises amino acids other than proline in positions 89 and 90 according to the Kabat numbering scheme.
35. The monoclonal antibody of any one of items 1 to 34, wherein the VL comprises A or a conservative substitution thereof in position 89; and/or G or a conservative substitution thereof in position 90, wherein the positions of the amino acids are indicated according to the Kabat numbering scheme.
36. The monoclonal antibody of any one of items 1 to 35, wherein the VL comprises A in position 89; and/or G in position 90, wherein the positions of the amino acids are indicated according to the Kabat numbering scheme.
37. The monoclonal antibody of any one of items 1 to 36, wherein the CDR-L3 of VL comprises or consists of positions 89, 90, 91, 92, 93, 94, 95, 95a, 95b, 95c, 96 and 97 according to the Kabat numbering scheme.
38. A monoclonal antibody specifically binding to L-thyroxine (T4), wherein said monoclonal antibody comprises:
   i) a heavy chain variable domain (VH) comprising (a) a CDR-H1 having the amino acid sequence of SEQ ID NO: 6 or a variant thereof having amino acid substitutions in 4 or less positions selected from positions 1, 2, 4, and 5 of SEQ ID NO: 6; (b) a CDR-H2 having the amino acid sequence of SEQ ID NO: 7 or a variant thereof having amino acid substitutions in 4 or less positions selected from positions 2 and 4 to 17 of SEQ ID NO: 7, and (c) a CDR-H3 having the amino acid sequence of SEQ ID NO: 8 or a variant thereof having amino acid substitutions in 4 or less positions selected from positions 1 to 3, 7, and 9 to 11 of SEQ ID NO: 8; and
   ii) a light chain variable domain (VL) comprising (d) a CDR-L1 having the amino acid sequence of SEQ ID NO: 9 or a variant thereof having amino acid substitutions in 4 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions in 4 or less positions selected from positions 1, 2, 6 to 8 and 12 of SEQ ID NO: 10.
39. A monoclonal antibody specifically binding to L-thyroxine (T4), wherein said monoclonal antibody comprises:
   i) a heavy chain variable domain (VH) comprising (a) a CDR-H1 having the amino acid sequence of SEQ ID NO: 6 or a variant thereof having amino acid substitutions in 4 or less positions selected from positions 1, 2, 4, and 5 of SEQ ID NO: 6; (b) a CDR-H2 having the amino acid sequence of SEQ ID NO: 7 or a variant thereof having amino acid substitutions in 4 or less positions selected from positions 2 and 4 to 17 of SEQ ID NO: 7, and (c) a CDR-H3 having the amino acid sequence of SEQ ID NO: 13 or a variant thereof having amino acid substitutions in 4 or less positions selected from positions 1 to 3, 7, and 9 to 11 of SEQ ID NO: 13; and
   ii) a light chain variable domain (VL) comprising (d) a CDR-L1 having the amino acid sequence of SEQ ID NO: 9 or a variant thereof having amino acid substitutions in 4 or less positions selected from positions 1 to 6, 9, 10, 12 and 13 of SEQ ID NO: 9 and (f) a CDR-L3 having the amino acid sequence of SEQ ID NO: 16 or a variant thereof having amino acid substitutions in 4 or less positions selected from positions 1, 2, 6 to 8 and 12 of SEQ ID NO: 16.
40. A monoclonal antibody specifically binding to L-thyroxine (T4), wherein said monoclonal antibody comprises:
   i) a heavy chain variable domain (VH) comprising (a) a CDR-H1 having the amino acid sequence of SEQ ID NO: 12 or a variant thereof having amino acid substitutions in 4 or less positions selected from positions 1, 2, 4, and 5 of SEQ ID NO: 12; (b) a CDR-H2 having the amino acid sequence of SEQ ID NO: 7 or a variant thereof having amino acid substitutions in 4 or less positions selected from positions 2 and 4 to 17 of SEQ ID NO: 7, and (c) a CDR-H3 having the amino acid sequence of SEQ ID NO: 14 or a variant thereof having amino acid substitutions in 4 or less positions selected from positions 1 to 3, 7, and 9 to 11 of SEQ ID NO: 14; and
   ii) a light chain variable domain (VL) comprising (d) a CDR-L1 having the amino acid sequence of SEQ ID NO: 9 or a variant thereof having amino acid substitutions in 4 or less positions selected from positions 1 to 6, 9, 10, 12 and 13 of SEQ ID NO: 9 and (f) a CDR-L3 having the amino acid sequence of SEQ ID NO: 17 or a variant thereof having amino acid substitutions in 4 or less positions selected from positions 1, 2, 6 to 8 and 12 of SEQ ID NO: 17.
41. A monoclonal antibody specifically binding to L-thyroxine (T4), wherein said monoclonal antibody comprises:
   i) a heavy chain variable domain (VH) comprising (a) a CDR-H1 having the amino acid sequence of SEQ ID NO: 12 or a variant thereof having amino acid substitutions in 4 or less positions selected from positions 1, 2, 4, and 5 of SEQ ID NO: 12; (b) a CDR-H2 having the amino acid sequence of SEQ ID NO: 7 or a variant thereof having amino acid substitutions in 4 or less positions selected from positions 2 and 4 to 17 of SEQ ID NO: 7, and (c) a CDR-H3 having the amino acid sequence of SEQ ID NO: 14 or a variant thereof having amino acid substitutions in 4 or less positions selected from positions 1 to 3, 7, and 9 to 11 of SEQ ID NO: 14; and
   ii) a light chain variable domain (VL) comprising (d) a CDR-L1 having the amino acid sequence of SEQ ID NO: 15 or a variant thereof having amino acid substitutions in 4 or less positions selected from positions 1 to 6, 9, 10, 12 and 13 of SEQ ID NO: 15 and (f) a CDR-L3 having the amino acid sequence of SEQ ID NO: 18 or a variant thereof having amino acid substitutions in 4 or less positions selected from positions 1, 2, 6 to 8 and 12 of SEQ ID NO: 18.
42. The monoclonal antibody of any one of items 38 to 41, wherein
   (a) the variant of SEQ ID NO: 6 has a M, L, I or V in position 4 and a N, S, T or Q in position 5;
   (b) the variant of SEQ ID NO: 7 has a I, A, L or V in position 2; a T or S in position 4; a R, G, D or K in position 5;
   (c) the variant of SEQ ID NO: 8 has a H, A, R or K in position 3 and a N, A or Q in position 7;
   (d) the variant of SEQ ID NO: 9 has a N, Q, S or T in position 9 and a A, N or V in position 10; and
   (e) the variant of SEQ ID NO:10 has a G, A or S in position 6; a S, G, N or Q in position 7; T, S or G in position 8; and V, A, I or L in position 12.
43. The monoclonal antibody of any one of items 38 to 41, wherein
   (a) the variant of SEQ ID NO: 6 has a M, L or I in position 4 and a N, S or T in position 5;
   (b) the variant of SEQ ID NO: 7 has a I, A or L in position 2; a T or S in position 4; a R, G, D or K in position 5;
   (c) the variant of SEQ ID NO: 13 has a H, A, R or K in position 3 and a N, A or Q in position 7;
   (d) the variant of SEQ ID NO: 9 has a N, Q, S or T in position 9 and a A, N or V in position 10; and
   (e) the variant of SEQ ID NO:16 has a G, A or S in position 6; a S, G or N in position 7; T, S or G in position 8; and V or A in position 12.
44. The monoclonal antibody of any one of items 38 to 41, wherein
   (a) the variant of SEQ ID NO: 12 has a M or L in position 4 and a N in position 5;
   (b) the variant of SEQ ID NO: 7 has a I in position 2; a T in position 4; a R, G or D in position 5;
   (c) the variant of SEQ ID NO: 14 has a H or A in position 3 and a N, A or Q in position 7;
   (d) the variant of SEQ ID NO: 9 has a N in position 9 and a A or N in position 10; and
   (e) the variant of SEQ ID NO:17 has a G, A or S in position 6; a S, G or N in position 7; T, S or G in position 8; and V or A in position 12.
45. The monoclonal antibody of any one of items 38 to 41, wherein
   (a) the variant of SEQ ID NO: 12 has a M in position 4 and a N in position 5;
   (b) the variant of SEQ ID NO: 7 has a I in position 2; a T in position 4; a R in position 5;
   (c) the variant of SEQ ID NO: 14 has a H in position 3 and a N in position 7;
   (d) the variant of SEQ ID NO: 9 has a N in position 9 and a A in position 10; and
   (e) the variant of SEQ ID NO:17 has a G in position 6; a S in position 7; T in position 8; and V in position 12.
46. The monoclonal antibody of any one of items 38 to 45, wherein the light chain variable domain comprises (e) a CDR-L2 having the amino acid sequence of SEQ ID NO: 11 or a variant thereof having amino acid substitutions in 4 or less positions of SEQ ID NO: 11.
47. The monoclonal antibody of any one of items 38 to 46, wherein the sum of the amino acid substitutions in the variants of CDRs H1, H2, H3, L1 and L3 is 12 or less.
48. The monoclonal antibody of item 46, wherein the sum of the amino acid substitutions in the variants of CDRs H1 H2, H3, L1, L2 and L3 is 13 or less.
49. The monoclonal antibody of any one of items 38 to 48, wherein said monoclonal antibody comprises:
   i) a heavy chain variable domain (VH) comprising (a) a CDR-H1 having the amino acid sequence of SEQ ID NO: 6 or a variant thereof having amino acid substitutions in 2 or less positions selected from positions 1, 2, 4, and 5 of SEQ ID NO: 6; (b) a CDR-H2 having the amino acid sequence of SEQ ID NO: 7 or a variant thereof having amino acid substitutions in 3 or less positions selected from positions 2 and 4 to 17 of SEQ ID NO: 7; and (c) a CDR-H3 having the amino acid sequence of SEQ ID NO: 8 or a variant thereof having amino acid substitutions in 2 or less positions selected from positions 1 to 3, 7, and 9 to 11 of SEQ ID NO: 8; and
   ii) a light chain variable domain (VL) comprising (d) a CDR-L1 having the amino acid sequence of SEQ ID NO: 9 or a variant thereof having amino acid substitutions in 2 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions in 3 or less positions selected from positions 1, 2, 6 to 8 and 12 of SEQ ID NO: 10.
50. The monoclonal antibody of any one of items 38 to 48, wherein said monoclonal antibody comprises:
   i) a heavy chain variable domain (VH) comprising (a) a CDR-H1 having the amino acid sequence of SEQ ID NO: 6 or a variant thereof having amino acid substitutions in 2 or less positions selected from positions 1, 2, 4, and 5 of SEQ ID NO: 6; (b) a CDR-H2 having the amino acid sequence of SEQ ID NO: 7 or a variant thereof having amino acid substitutions in 3 or less positions selected from positions 2 and 4 to 17 of SEQ ID NO: 7; and (c) a CDR-H3 having the amino acid sequence of SEQ ID NO: 13 or a variant thereof having amino acid substitutions in 2 or less positions selected from positions 1 to 3, 7, and 9 to 11 of SEQ ID NO: 13; and
   ii) a light chain variable domain (VL) comprising (d) a CDR-L1 having the amino acid sequence of SEQ ID NO: 9 or a variant thereof having amino acid substitutions in 2 or less positions selected from positions 1 to 6, 9, 10, 12 and 13 of SEQ ID NO: 9 and (f) a CDR-L3 having the amino acid sequence of SEQ ID NO: 16 or a variant thereof having amino acid substitutions in 3 or less positions selected from positions 1, 2, 6 to 8 and 12 of SEQ ID NO: 16.
51. The monoclonal antibody of any one of items 38 to 48, wherein said monoclonal antibody comprises:
   i) a heavy chain variable domain (VH) comprising (a) a CDR-H1 having the amino acid sequence of SEQ ID NO: 12 or a variant thereof having amino acid substitutions in 2 or less positions selected from positions 1, 2, 4, and 5 of SEQ ID NO: 12; (b) a CDR-H2 having the amino acid sequence of SEQ ID NO: 7 or a variant thereof having amino acid substitutions in 3 or less positions selected from positions 2 and 4 to 17 of SEQ ID NO: 7; and (c) a CDR-H3 having the amino acid sequence of SEQ ID NO: 14 or a variant thereof having amino acid substitutions in 2 or less positions selected from positions 1 to 3, 7, and 9 to 11 of SEQ ID NO: 14; and
   ii) a light chain variable domain (VL) comprising (d) a CDR-L1 having the amino acid sequence of SEQ ID NO: 9 or a variant thereof having amino acid substitutions in 2 or less positions selected from positions 1 to 6, 9, 10, 12 and 13 of SEQ ID NO: 9 and (f) a CDR-L3 having the amino acid sequence of SEQ ID NO: 17 or a variant thereof having amino acid substitutions in 3 or less positions selected from positions 1, 2, 6 to 8 and 12 of SEQ ID NO: 17.
52. The monoclonal antibody of any one of items 38 to 48, wherein said monoclonal antibody comprises:
   i) a heavy chain variable domain (VH) comprising (a) a CDR-H1 having the amino acid sequence of SEQ ID NO: 12 or a variant thereof having amino acid substitutions in 2 or less positions selected from positions 1, 2, 4, and 5 of SEQ ID NO: 12; (b) a CDR-H2 having the amino acid sequence of SEQ ID NO: 7 or a variant thereof having amino acid substitutions in 3 or less positions selected from positions 2 and 4 to 17 of SEQ ID NO: 7; and (c) a CDR-H3 having the amino acid sequence of SEQ ID NO: 14 or a variant thereof having amino acid substitutions in 2 or less positions selected from positions 1 to 3, 7, and 9 to 11 of SEQ ID NO: 14; and
   ii) a light chain variable domain (VL) comprising (d) a CDR-L1 having the amino acid sequence of SEQ ID NO: 15 or a variant thereof having amino acid substitutions in 2 or less positions selected from positions 1 to 6, 9, 10, 12 and 13 of SEQ ID NO: 15 and (f) a CDR-L3 having the amino acid sequence of SEQ ID NO: 18 or a variant thereof having amino acid substitutions in 3 or less positions selected from positions 1, 2, 6 to 8 and 12 of SEQ ID NO: 18.
53. The monoclonal antibody of any one of items 38 to 52, wherein the light chain variable domain comprises (e) a CDR-L2 having the amino acid sequence of SEQ ID NO: 11 or a variant thereof having amino acid substitution in 1 or less positions of SEQ ID NO: 11.
54. The monoclonal antibody of any one of items 38 to 53, wherein said monoclonal antibody comprises:
   i) a heavy chain variable domain (VH) comprising (a) a CDR-H1 having the amino acid sequence of SEQ ID NO: 6 or a variant thereof having amino acid substitutions in 2 or less positions selected from positions 1 and 4 of SEQ ID NO: 6; (b) a CDR-H2 having the amino acid sequence of SEQ ID NO:7 or a variant thereof having amino acid substitutions in 3 or less positions selected from positions 5, 8 and 13 of SEQ ID NO: 7, and (c) a CDR-H3 having the amino acid sequence of SEQ ID NO: 8 or a variant thereof having amino acid substitutions in 2 or less positions selected from positions 3 and 7 of SEQ ID NO: 8; and
   ii) a light chain variable domain (VL) comprising (d) a CDR-L1 having the amino acid sequence of SEQ ID NO: 9 or a variant thereof having amino acid substitutions in 2 or less positions selected from positions 8 and 10 of SEQ ID NO: 9 and (f) a CDR-L3 having the amino acid sequence of SEQ ID NO: 10 or a variant thereof having amino acid substitutions in 3 or less positions selected from positions 4, 5 and 6 of SEQ ID NO: 10.
55. The monoclonal antibody of any one of items 38 to 53, wherein said monoclonal antibody comprises:
   i) a heavy chain variable domain (VH) comprising (a) a CDR-H1 having the amino acid sequence of SEQ ID NO: 6 or a variant thereof having amino acid substitutions in 2 or less positions selected from positions 1 and 4 of SEQ ID NO: 6; (b) a CDR-H2 having the amino acid sequence of SEQ ID NO:7 or a variant thereof having amino acid substitutions in 3 or less positions selected from positions 5, 8 and 13 of SEQ ID NO: 7, and (c) a CDR-H3 having the amino acid sequence of SEQ ID NO: 13 or a variant thereof having amino acid substitutions in 2 or less positions selected from positions 3 and 7 of SEQ ID NO: 13; and
   ii) a light chain variable domain (VL) comprising (d) a CDR-L1 having the amino acid sequence of SEQ ID NO: 9 or a variant thereof having amino acid substitutions in 2 or less positions selected from positions 8 and 10 of SEQ ID NO: 9 and (f) a CDR-L3 having the amino acid sequence of SEQ ID NO: 16 or a variant thereof having amino acid substitutions in 3 or less positions selected from positions 4, 5 and 6 of SEQ ID NO: 16.
56. The monoclonal antibody of any one of items 38 to 53, wherein said monoclonal antibody comprises:
   i) a heavy chain variable domain (VH) comprising (a) a CDR-H1 having the amino acid sequence of SEQ ID NO: 12 or a variant thereof having amino acid substitutions in 2 or less positions selected from positions 1 and 4 of SEQ ID NO: 6; (b) a CDR-H2 having the amino acid sequence of SEQ ID NO:7 or a variant thereof having amino acid substitutions in 3 or less positions selected from positions 5, 8 and 13 of SEQ ID NO: 7, and (c) a CDR-H3 having the amino acid sequence of SEQ ID NO:14 or a variant thereof having amino acid substitutions in 2 or less positions selected from positions 3 and 7 of SEQ ID NO: 14; and
   ii) a light chain variable domain (VL) comprising (d) a CDR-L1 having the amino acid sequence of SEQ ID NO: 9 or a variant thereof having amino acid substitutions in 2 or less positions selected from positions 8 and 10 of SEQ ID NO: 9 and (f) a CDR-L3 having the amino acid sequence of SEQ ID NO: 17 or a variant thereof having amino acid substitutions in 3 or less positions selected from positions 4, 5 and 6 of SEQ ID NO: 17.
57. The monoclonal antibody of any one of items 38 to 53, wherein said monoclonal antibody comprises:
   i) a heavy chain variable domain (VH) comprising (a) a CDR-H1 having the amino acid sequence of SEQ ID NO: 12 or a variant thereof having amino acid substitutions in 2 or less positions selected from positions 1 and 4 of SEQ ID NO: 6; (b) a CDR-H2 having the amino acid sequence of SEQ ID NO:7 or a variant thereof having amino acid substitutions in 3 or less positions selected from positions 5, 8 and 13 of SEQ ID NO: 7, and (c) a CDR-H3 having the amino acid sequence of SEQ ID NO:14 or a variant thereof having amino acid substitutions in 2 or less positions selected from positions 3 and 7 of SEQ ID NO: 14; and
   ii) a light chain variable domain (VL) comprising (d) a CDR-L1 having the amino acid sequence of SEQ ID NO: 15 or a variant thereof having amino acid substitutions in 2 or less positions selected from positions 8 and 10 of SEQ ID NO: 15 and (f) a CDR-L3 having the amino acid sequence of SEQ ID NO: 18 or a variant thereof having amino acid substitutions in 3 or less positions selected from positions 4, 5 and 6 of SEQ ID NO: 18.
58. The monoclonal antibody of any one of items 38 to 57, wherein the light chain variable domain comprises (e) a CDR-L2 having the amino acid sequence of SEQ ID NO: 11 or a variant thereof having 1 or less amino acid substitution in position 6 of SEQ ID NO: 11.
59. The monoclonal antibody of any one of items 38 to 58, wherein each of the amino acid substitutions are conservative amino acid substitutions, preferably highly conservative amino acid substitutions.
60. The monoclonal antibody of any one of items 1 to 59, wherein the heavy chain variable domain (VH) comprises (a) a CDR-H1 comprising the amino acid sequence of X1NVX2N (wherein X1 is S or R and X2 is M or L); (b) a CDR-H2 comprising the amino acid sequence of SEQ ID NO: 20, and (c) a CDR-H3 comprising the amino acid sequence of SEQ ID NO: 21; and wherein the light chain variable domain (VL) comprises (d) a CDR-L1 comprising the amino acid sequence of SEQ ID NO: 22 and (f) a CDR-L3 comprising the amino acid sequence of SEQ ID NO: 23.
61. The monoclonal antibody of any one of items 1 to 60, wherein the light chain variable domain (VL) comprises a CDR-L2 comprising the amino acid sequence of SEQ ID NO: 24.
62. The monoclonal antibody of any one of items 1 to 61, wherein the heavy chain variable domain (VH) comprises (a) a CDR-H1 comprising or consisting of an amino acid sequence selected from the group consisting of SEQ ID NOs: 12, 25 and 26 (b) a CDR-H2 comprising or consisting of an amino acid sequence selected from the group consisting of SEQ ID NOs: 7, 27 and 28, and (c) a CDR-H3 comprising or consisting of an amino acid sequence selected from the group consisting of SEQ ID NOs: 14 and 29 and wherein the light chain variable domain (VL) comprises (d) a CDR-L1 comprising or consisting of an amino acid sequence selected from the group consisting of SEQ ID NOs: 15 and 30 and (f) a CDR-L3 comprising or consisting of an amino acid sequence selected from the group consisting of SEQ ID NOs: 18, 31 and 32.
63. The monoclonal antibody of any one of items 1 to 62, wherein the light chain variable domain (VL) comprises a CDR-L2 comprising the amino acid sequence of SEQ ID NO: 11 or 33.
64. The monoclonal antibody of any one of items 1 to 63, wherein the heavy chain variable domain (VH) comprises framework regions (FW) flanking the CDRs of the VH as represented in formula I:

FW-H1-CDR-H1-FW-H2-CDR-H2-FW-H3-CDR-H3-FW-H4 (formula I); and
wherein FW-H1 has the amino acid sequence of SEQ ID NO: 34 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 34;
wherein FW-H2 has the amino acid sequence of SEQ ID NO: 35 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 35;
wherein FW-H3 has the amino acid sequence of SEQ ID NO: 36 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 36; and
wherein FW-H4 has the amino acid sequence of SEQ ID NO: 37 or a variant thereof having at least 60%, preferably at least 70%, even more preferably at least 80%, even more preferably at least 85%, even more preferably at least 90%, even more preferably at least 95%, even more preferably at least 99% sequence identity SEQ ID NO: 37.
65. The monoclonal antibody of any one of items 1 to 64, wherein the light chain variable domain (VL) comprises framework regions (FW) flanking the CDRs of the VL as represented in formula I:

FW-L1-CDR-L1-FW-L2-CDR-L2-FW-L3-CDR-L3-FW-L4 (formula I); and
wherein FW-L1 has the amino acid sequence of SEQ ID NO: 38 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 38;
wherein FW-L2 has the amino acid sequence of SEQ ID NO: 39 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 39;
wherein FW-L3 has the amino acid sequence of SEQ ID NO: 40 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 40; and
wherein FW-L4 has the amino acid sequence of SEQ ID NO: 41 or a variant thereof having at least 60%, preferably at least 70%, even more preferably at least 80%, even more preferably at least 85%, even more preferably at least 90%, even more preferably at least 95%, even more preferably at least 99% sequence identity SEQ ID NO: 41.
66. The monoclonal antibody of any one of items 1 to 65, wherein the VH has the amino acid sequence of SEQ ID NO: 42 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 42.
67. The monoclonal antibody of any one of items 1 to 66, wherein the VH has the amino acid sequence of SEQ ID NO: 43 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 43.
68. The monoclonal antibody of any one of items 1 to 67, wherein the VH has the amino acid sequence of SEQ ID NO: 44 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 44.
69. The monoclonal antibody of any one of items 1 to 68, wherein the VL has the amino acid sequence of SEQ ID NO: 45 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 45.
70. The monoclonal antibody of any one of items 1 to 69, wherein the VL has the amino acid sequence of SEQ ID NO: 46 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 46.
71. The monoclonal antibody of any one of items 1 to 70, wherein the VL has the amino acid sequence of SEQ ID NO: 47 or a variant thereof having at least 60%, preferably at least 70%, even more preferably at least 80%, even more preferably at least 85%, even more preferably at least 90%, even more preferably at least 95%, even more preferably at least 99% sequence identity to SEQ ID NO: 47.
72. The monoclonal antibody of any one of items 1 to 71, wherein at a temperature of 37° C. the association rate constant (kₐ) of the monoclonal antibody with T4 is 1.9*10⁷ M⁻¹sec⁻¹ or higher, in embodiments 2*10⁷ M⁻¹sec⁻¹ or higher, in embodiments 1*108 M⁻¹sec⁻¹ or higher, in embodiments 3*108 M⁻¹sec⁻¹ or higher and in embodiments 1*10⁹ M⁻¹ sec⁻¹ or higher.
73. The monoclonal antibody of any one of items 1 to 72, wherein the association rate constant (kₐ) for the binding to T4 corresponds to at least 10%, preferably at least 20%, even more preferably at least 30%, even more preferably at least 50%, even more preferably at least 70%, even more preferably at least 80% and even more preferably at least 90% of the association rate constant (kₐ) for the binding to T4 of the antibody comprising or consisting of a heavy chain of SEQ ID NO: 48 and a light chain of SEQ ID NO: 49, wherein the association rate constants are measured under the identical experimental conditions.
74. The monoclonal antibody of item 72 or 73, wherein the kₐ is determined by surface plasmon resonance spectroscopy.
75. The monoclonal antibody of item 74, wherein the surface plasmon resonance spectroscopy is analyzed using correction for mass transport limitation.
76. The monoclonal antibody of any one of items 1 to 73, wherein the monoclonal antibody binds T4 with a K_{D} of 20 nm or less, in embodiments 10 nM or less, in embodiments 5 nM or less, in embodiments 1 nM or less, in embodiments 0.5 nM or less, in embodiments 0.3 nM or less and in embodiments 0.28 nM or less.
77. The monoclonal antibody of item 76, wherein the K_{D} is determined by surface plasmon resonance spectroscopy, optionally using affinity in solution measurement.
78. The monoclonal antibody of any one of items 1 to 77, wherein the monoclonal antibody discriminates T4 from 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine.
79. The monoclonal antibody of any one of items 1 to 78, wherein the monoclonal antibody discriminates T4 from L-T3 and/or rT3.
80. The monoclonal antibody of item 78 or 79, wherein discriminating T4 from the respective substance(s) means that the K_{D} for binding to T4 is at least 4-fold lower, in embodiments at least 5-fold lower than the K_{D} for the other substance(s), respectively.
81. The monoclonal antibody of any one of items 1 to 80, wherein the monoclonal antibody when used in a competitive immunoassay for quantifying T4 (e.g. fT4) shows a signal-to-noise ratio that is at least 29%, in embodiments 65% and in embodiments at least 95% of the signal-to noise ratio achieved with a Fab fragment having a heavy chain sequence of SEQ ID NO: 48 and a light chain sequence of SEQ ID NO: 49 in the otherwise identical immunoassay setup.
82. The monoclonal antibody of any one of items 1 to 81, wherein the monoclonal antibody is a Fab fragment.
83. A polynucleotide encoding
   (i) the heavy chain or heavy chain variable domain of the monoclonal antibody according to any one of items 1 to 82, and/or
   (ii) the light chain or light chain variable domain of the monoclonal antibody according to any one of items 1 to 82.
84. A vector comprising the polynucleotide according to item 83.
85. A host cell comprising the polynucleotide according to item 83, or the vector according to item 84.
86. The host cell according to item 85 that is a prokaryotic cell or a eukaryotic cell.
87. The host cell according to item 85 that is a eukaryotic cell, wherein said cell is a HEK or a CHO cell.
88. A method of producing the monoclonal antibody according to any one of items 1 to 82 comprising culturing the host cell according to any one of items 85 to 87 and isolating said antibody.
89. An antibody according to any one of items 1 to 82 obtainable by the method of item 88.
90. A composition comprising the antibody according to any one of items 1 to 82, the polynucleotide according to item 83, the vector according to item 84, or the host cell according to any one of items 85 to 87.
91. A composition comprising the antibody according to any one of items 1 to 82 that is a diagnostic composition.
92. Use of the antibody according to any one of items items 1 to 82 or the composition according to item 90 or 91 for an in vitro detection, preferably in vitro quantification, of T4 (e.g. fT4) in a sample, preferably using an immunoassay.
93. The use according to item 92, wherein the immunoassay is a heterogeneous immunoassay.
94. The use according to item 92 or 93, wherein the sample is blood, preferably plasma or serum.
95. The use according to any one of items 92 to 94, wherein the immunoassay is a competitive immunoassay.
96. An in vitro immunoassay method for quantifying T4 (e.g. fT4) in a sample using the antibody as defined in any one of items 1 to 82.
97. The method of item 96, wherein the sample is a body fluid.
98. The method of item 97, wherein the body fluid is a blood sample, cerebrospinal fluid, seminal fluid, saliva or urine.
99. The method of item 96 or 97, wherein the body fluid is a blood sample that is whole blood, serum or plasma.
100. The method of any one of items 96 to 99, wherein the method comprises (i) incubating a sample comprising T4 (e.g. fT4) with the antibody as defined in any one of items 1 to 82 or a composition as defined in item 90 or 91; and (ii) quantifying the amount of T4 (e.g. fT4) in the sample by directly or indirectly detecting the T4 (e.g. fT4) bound to the antibody or the antibody comprised in the composition, respectively.
101. The method of any one of items 96 to 100, wherein the method comprises (i) incubating a sample comprising T4 (e.g. fT4) with the antibody as defined in any one of items 1 to 82 or a composition as defined in item 90 or 91, wherein the antibody has a detection label attached thereto; and (ii) quantifying the amount of T4 (e.g. fT4) in the sample by quantifying the amount of the antibody not bound to the T4 (e.g. fT4) in the sample via the detection label.
102. The method of item 101, wherein the detection label is selected from an enzyme, a label emitting light, in an embodiment fluorescence, luminescence, chemiluminescence, electrochemiluminescence or radioactivity.
103. The method of any one of items 96 to 102, wherein the method is a method for detecting total T4 in a sample, and wherein the method comprises treatment of the sample with a reagent for releasing T4 from binding proteins (e.g. serum proteins), wherein in embodiments the reagent for releasing T4 from its binding protein is 8-Anilinonaphthalene-1-sulfonic acid (ANS).
104. A kit comprising the antibody as defined in any one of items 1 to 82 or the composition as defined in item 90 or 91.
105. The kit according to item 103, wherein the kit is a kit for detecting and/or quantifying T4 (e.g. fT4) in vitro.
106. The kit according to item 103 or 104, wherein the kit is an immunoassay kit.

The present invention provides antibodies specifically binding L-thyroxine. (referred herein as L-T4 or T4). Alternative names for L-thyroxine used in the art are 3,3′,5,5″-Tetraiodo-L-thyronine and 3-[4-(4-Hydroxy-3,5-diiodophenoxy)-3,5-diiodophenyl]-L-alanine. T4 has CAS No. 51-48-9. T4 circulates in the bloodstream as an equilibrium mixture of free and serum bound hormone. Free T4 (fT4) is the unbound and biologically active form, which represents only 0.03% of the total T4. The remaining T4 is inactive and bound to serum proteins such as thyroxine binding globulin (TBG) (75%), pre-albumin (15%), and albumin (10%) (Robbins J, Rall J E. Recent Prog Horm Res 1957; 13:161-208; Oppenheimer J H. N Engl J Med 1968; 278(21):1153-1162; DeGroot L J, Larsen P R, Hennemann G. Wiley and Sons, New York, 1984:62-65; Ekins R P. Endocr Rev 1990; 11(1):5-46). As T4 bound to serum proteins is hardly accessible for antibody binding, the antibodies of the invention, it is evident that an antibody specifically binding T4/L-T4 binds to T4/L-T4 in its free form (i.e. not binding protein bound form). Accordingly, in embodiments the antibodies of the invention may also specifically bind to fT4. In embodiments, specifically binding to fT4 does not mean that fT4 can be discriminated from serum protein bound T4, but relates to the discrimination of T4 from related substances/derivates such as L-T3 and others as disclosed herein elsewhere. The antibodies of the invention may in embodiments be for detection of fT4. In other embodiments, the antibodies can be used for detection of total T4 in a sample. In the latter embodiment, the use of the antibody typically includes a pretreatment of a sample to release T4 bound to proteins (e.g. serum proteins).

The terms “antibody”, “antibodies”, and analogous terms as used herein relate to full immunoglobulin molecules and encompass naturally-occurring forms of antibodies (including but not limited to IgG, IgA, IgM, IgE) as well as recombinant antibody constructs including but not limited to single-chain antibodies, chimeric antibodies, humanized antibodies, antibody-fusion proteins, and multi-specific antibodies; as well as antigen binding fragments and derivatives of all of the foregoing. The terms “antibody”, “antibodies”, and analogous terms as used herein also refer to antigen binding fragments thereof, which may be referenced herein as antibody antigen binding fragment, and/or, simply antigen binding fragment. These terms refer to one or more fragments of an antibody that retain the ability to specifically bind to the target antigen, i.e. L-T4, as known in the art, including but not limited to antigen binding fragments comprising an Fv domain, i.e., paired heavy and light chain variable domains, such as Fab, Fab′, F(ab′)₂, and Fv fragments as well as recombinant constructs such as single-chain Fv domains, known in the art as scFvs. The terms also includes antibody antigen binding fragments that comprise a single, unpaired heavy or light chain variable domain as known in the art that retains the ability to specifically and selectively bind antigen as defined herein, including but not limited to single domain antibodies (also referenced in the art as sdAbs, dAbs, and/or nanobodies) and V_{H}H domains based on the heavy chains of camelids.

In certain embodiments the monoclonal antibody of the invention may be a full-immunoglobulin, Fab, Fab′, F(ab′)₂, Fv or scFv. In a specific embodiment, the monoclonal antibody of the invention may be a Fab fragment.

Antibodies may be polyclonal or monoclonal. The antibodies of the invention are monoclonal. The term “monoclonal as used herein with reference to an antibody or antigen binding fragment thereof, refer to a population of antibody polypeptides or fragments thereof produced from a single B cell clone, which population contains only one species of an antigen binding site capable of immunoreacting with a particular epitope of an antigen. This is in contrast with “polyclonal” antibodies and compositions, which term(s) refer to a population of antibody polypeptides or antigen binding fragments that contain multiple species of antigen binding sites. Also included are modified forms of monoclonal antibodies of the invention such as humanized or chimeric versions thereof, as well as recombinant antibody constructs, such as antibody (or antigen binding fragment)-fusion proteins, wherein the antibody or antigen binding fragment comprises (an) additional domain(s), e.g. for the isolation and/or preparation of recombinantly produced antibody/fragment/constructs.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “paratope” as used herein in the context of antibodies relates to the amino acids of the VH and VL that directly interact with the antigen (e,g, L-T4). Paratope amino acids may interact with the antigen via different interaction modes. Exemplary interaction modes are hydrophobic interaction, H-bond, H-bond with H₂O coordination and H₂O coordination. The interaction of each paratope residue with the antigen may individually be mediated via the amino acid side chain or the amino acid backbone.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The numbering of amino acid residues of the VH and VL of the antibody of the invention is made according to Kabat nomenclature if not specifically mentioned otherwise. A skilled person can convert the numbering according to Kabat into other nomenclatures such as Clothia, McCallum, etc.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “substitution”, “exchange” or “mutate” as used herein in the context of amino acids refers to the replacement of an amino acid with another amino acid. The deletion of an amino acid at a certain position and the introduction of one (or more) amino acid(s) at a different position is explicitly not encompassed by the term “substitution”. As noted, the present invention encompasses conservative or highly conservative amino acid substitutions as have been defined herein above.

In some instances, herein it is defined that a certain amino acid is selected from a subset of two or more amino acids or a conservative (or highly conservative) substitution thereof. In these instances, the respective amino acid position may be any of the listed amino acids or a conservative (or highly conservative) amino acid substitution of any of the explicitly listed amino acids. What a conservative and highly conservative substitution as used herein means is defined elsewhere herein. To give an illustrative example, if a certain amino acid is defined as “A or T or a conservative amino substitution thereof”, the amino acid can be A or any conservative amino acid exchange thereof or T or a conservative amino substitution thereof.

Amino acids are herein either spelled out or abbreviated using a 1-letter code or a three letter code.

In the context of the invention, it is referred to variants of sequences (in particular CDRs). These variants typically comprise one or more amino acid substitutions. It is evident that the variant CDRs are functional variants, i.e. having amino acid sequences that may differ from the reference amino acid sequence but which differing sequence exhibits or maintains the same functional activity as the reference sequence in the context of the described heavy and/or light chain variable domain. Specifically, as used herein, the term same functional activity means that the antibody or antibody binding fragment of the invention comprising one or more variant CDRs will maintain the extraordinary high association rate constant kₐ and/or the affinity with respect to the binding to L-T4, the specificity (in particular with respect to the discrimination of L-T4 from structurally related compounds) and/or the high signal to noise ratio in an immunoassay are maintained.

As used in the context of the invention, a “conservative amino acid substitution” means the substitution of an amino acid with another amino acid selected from its same physicochemical group, wherein the physicochemical groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu
   wherein if Cys is to be conservatively substituted, it is substituted with Ser or Ala, and
   wherein if Pro is to be conservatively substituted it is substituted with Ala.

As used in the context of the invention, a “highly conservative amino acid substitution” means the following amino acid substitutions:
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

As used herein, the term “% sequence identity” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

As used herein, “nucleic acid molecule”, “nucleic acid sequence”, “polynucleotide” and analogous terms include both genomic DNA and cDNA, as well as RNA capable of driving expression of an antibody or antigen binding fragment of the invention. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. In embodiments, the nucleic acids/nucleic acid sequences of the invention may be isolated. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules.

“Immunoassays” as used herein are well-established bioanalytical methods in which detection or quantitation of an analyte depends on the reaction of the analyte and at least one analyte-specific binding agent, thus forming an analyte:binding agent complex. In the context of the present invention, at least one of the at least one analyte specific binding agent is an antibody of the invention. The specific embodiment of a “sandwich” immunoassay can be used for analytes possessing more than one recognition epitopes. Thus, a sandwich assay requires at least two binding agents that attach to non-overlapping epitopes on the analyte. In a “heterogeneous sandwich immunoassay” one of the binding agents has the functional role of an analyte-specific capture binding agent; this binding agent is or (during the course of the assay) becomes immobilized on a solid phase. A second analyte-specific binding agent is supplied in dissolved form in the liquid phase. A sandwich-like complex is formed once the respective analyte is bound by a first and a second binding agent (binding agent-1:analyte:binding agent-2). The sandwich-like complex is also referred to as “detection complex”. Within the detection complex the analyte is sandwiched between the binding agents, i.e. in such a complex the analyte represents a connecting element between the first binding agent and a second binding agent.

The term “heterogeneous” (as opposed to “homogeneous”) denotes two essential and separate steps in the assay procedure. In the first step, a detection complex containing label is formed and immobilized, however with unbound label still surrounding the complexes. Prior to determination of a label-dependent signal unbound label is removed from the immobilized detection complex, thus representing the second step. In contrast, a homogeneous assay produces an analyte-dependent detectable signal by way of single-step incubation and does not require a washing step.

In a heterogeneous immunoassay the solid phase is functionalized such that it may have bound to its surface the functional capture binding agent (the first binding agent), prior to being contacted with the analyte; or the surface of the solid phase is functionalized in order to be capable of anchoring a first binding agent, after it has reacted with the analyte. In the latter case, the anchoring process must not interfere with the binding agent's ability to specifically capture and bind the analyte. A second binding agent present in the liquid phase is used for detection of bound analyte. Thus, in a heterogeneous immunoassay the analyte is allowed to bind to the first (capture) and second (detector) binding agents. Thereby a “detection complex” is formed wherein the analyte is sandwiched between the capture binding agent and the detector binding agent. In a typical embodiment, the detector binding agent is labeled prior to being contacted with the analyte; alternatively a label is specifically attached to the detector binding agent after analyte binding. With the detection complexes being immobilized on the solid phase, the amount of label detectable on the solid phase corresponds to the amount of sandwiched analyte. After removal of unbound label, immobilized label indicating presence and amount of analyte can be detected.

A “competitive immunoassay” as used herein preferably employs a single binding agent directly interacting with the analyte (i.e. T4/fT4). A “competitive heterogeneous immunoassay typically detects a signal of a detection label that inversely corresponds to the amount of analyte in a sample. In preferred embodiments herein, the competitive immunoassay may be a heterogeneous competitive immunoassay. In embodiments, the sample with the analyte is mixed with an artificially produced labeled analogon of the analyte that is capable of reacting with the analyte-specific binding agent (e.g. antibody of the invention). In the assay, the analyte and the analogon compete for binding to a capture binding agent (e.g. the antibody of the invention) which is or becomes immobilized. The amount of binding agent is selected to be limiting in this setting. Following the binding step, the higher the amount of immobilized label, the smaller the amount of the non-labeled analyte that was capable of competing for the capture binding agent. Immobilized label is determined after a washing step. In this setting, the amount of label that is detectable on the solid phase inversely corresponds to the amount of analyte that was initially present in the sample. In other embodiments, the competitive immunoassay may be a heterogeneous back titration assay. In such assay, the sample comprising the analyte is first incubated with a binding agent (e.g. the antibody of the invention) having a detection label attached for a time sufficient to form a analyte:binding agent complex. The binding agent may be provided in excess to the highest analyte concentration to be measured. Subsequently, a capture-analogon (e.g. biotinylated L-T4) that competes for binding to the binding agent having the detection label (e.g. competes with the T4 (e.g. fT4) in the sample for the antibody of the invention) is added to the mixture. The capture analogon is immobilized or becomes immobilized (e.g. via a biotin-streptavidin interaction) to a surface (e.g. of magnetic beads) during the incubation with the mixture. The amount of the analyte can then be detected by removing the non-surface bound reagents and detecting the signal of the detection label. The more detection label (attached to the binding agent) is found on the surface, the less analyte was present in the sample. Accordingly, also in this second setting the amount of label that is detectable on the solid phase inversely corresponds to the amount of analyte that was initially present in the sample.

“Detectable labels” as used herein relates to labels that allow for detection. According to an embodiment of the current invention a detectable label is an enzyme, or a label emitting light, in an embodiment fluorescence, luminescence, chemiluminescence, electro-,chemiluminescence or radioactivity. In a preferred embodiment the label is an electrochemiluminescent label, in an embodiment Tris(2,2′-bipyridyl)ruthenium(II)-complex (Ru(bpy)). As the interference is caused by the three-dimensional structure of the label molecule that attracts auto-antibodies and similar interfering molecules and not by the signal-emitting mechanism of said label, such as e.g. light or radioactivity, all the above-referenced labels can be used in the current invention.

“Capture labels” as used herein relates to labels that can immobilize a capture agent (e.g. T4 having a capture label attached thereto) on a surface (e.g., on a magnetic particle such as a microbead). Non-limiting examples are members of binding pairs. A non-limiting example for a capture label is biotin or derivatives thereof, which can interact with streptavidin or derivatives thereof. Different capture labels are well known in the art.

A “sample” as used in the context of the present disclosure may be a liquid sample comprising or expected to comprise L_T4. The sample may in particular be a body fluid, such as, but not restricted to a blood sample, cerebrospinal fluid, seminal fluid, saliva or urine. In embodiments, the sample is a blood sample, such as whole blood, serum or plasma. In embodiments, the sample is serum or plasma.

The word “comprise”, and variations such as “comprises” and “comprising”, is to be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

All amino acid sequences provided herein are presented starting with the most N-terminal residue and ending with the most C-terminal residue, as customarily done in the art, and the one-letter or three-letter code abbreviations as used to identify amino acids throughout the present invention correspond to those commonly used for amino acids.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In addition to the aspects and embodiments described and provided elsewhere in the present disclosure, the following non-limiting list of embodiments are also contemplated.
1. A monoclonal antibody specifically binding to L-thyroxine (T4), wherein said monoclonal antibody comprises:
   i) a heavy chain variable domain (VH) comprising V or A in position 33; Y in position 50; W in position 52; I in position 98, G, A or V in position 99; Y in position 100; and I in position 100b; and
   ii) a light chain variable domain (VL) comprising amino acids H or Y in position 28; N or K in position 29; W in position 32; G or A in position 91; Y, W or F in position 92; S or T in position 93; Y or F in position 95b; N, S, T or Q in position 95c; and H in position 96,
   wherein the positions of the amino acids in the VH and the VL are indicated according to the Kabat numbering scheme, respectively.
2. The monoclonal antibody of clause 1, wherein the VH comprises M, L, I or V in position 34; N, S, T or Q in position 35; I, A, L or V in position 51; T or S in position 52a; R, G, D or K in position 53; H, A, R or K in position 97; and/or N, A or Q in position 100a, wherein the positions of the amino acids are indicated according to the Kabat numbering scheme; and/or wherein the VL comprises N, Q, S or T in position 30; A, N or V in position 31; G, A or S in position 94; S, G, N, Q in position 95; T, S or G in position 95a; and/or V, A, I or L in position 97, wherein the positions of the amino acids are indicated according to the Kabat numbering scheme.
3. A monoclonal antibody specifically binding to L-thyroxine (T4), wherein said monoclonal antibody comprises:
   i) a heavy chain variable domain (VH) comprising (a) a CDR-H1 having the amino acid sequence of SEQ ID NO: 6 or a variant thereof having amino acid substitutions in 4 or less positions selected from positions 1, 2, 4, and 5 of SEQ ID NO: 6; (b) a CDR-H2 having the amino acid sequence of SEQ ID NO: 7 or a variant thereof having amino acid substitutions in 4 or less positions selected from positions 2 and 4 to 17 of SEQ ID NO: 7, and (c) a CDR-H3 having the amino acid sequence of SEQ ID NO: 8 or a variant thereof having amino acid substitutions in 4 or less positions selected from positions 1 to 3, 7, and 9 to 11 of SEQ ID NO: 8; and
   ii) a light chain variable domain (VL) comprising (d) a CDR-L1 having the amino acid sequence of SEQ ID NO: 9 or a variant thereof having amino acid substitutions in 4 or less positions selected from positions 1 to 6, 9, 10, 12 and 13 of SEQ ID NO: 9 and (f) a CDR-L3 having the amino acid sequence of SEQ ID NO: 10 or a variant thereof having amino acid substitutions in 4 or less positions selected from positions 1, 2, 6 to 8 and 12 of SEQ ID NO: 10.
4. The monoclonal antibody of clause 3, wherein the light chain variable domain comprises a CDR-L2 having the amino acid sequence of SEQ ID NO: 11 or a variant thereof having amino acid substitutions in 4 or less positions of SEQ ID NO: 11.
5. The monoclonal antibody of any one of clauses 1 to 4, wherein at a temperature of 37° C. the association rate constant (kₐ) of the monoclonal antibody with T4 is 1.9*10⁷ M⁻¹ sec⁻¹ or higher.
6. The monoclonal antibody of any one of clauses 1 to 5, wherein the association rate constant (kₐ) for the binding to T4 corresponds to at least 10% of the association rate constant (kₐ) for the binding to T4 of the antibody comprising or consisting of a heavy chain of SEQ ID NO: 48 and a light chain of SEQ ID NO: 49, wherein the association rate constants are measured under the identical experimental conditions.
7. The monoclonal antibody of any one of clauses 1 to 6, wherein the monoclonal antibody discriminates T4 from 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine.
8. The monoclonal antibody of any one of clauses 1 to 7, wherein the monoclonal antibody when used in a competitive immunoassay for quantifying T4 shows a signal-to-noise ratio that is at least 29%, in embodiments 65% and in embodiments at least 95% of the signal-to noise ratio achieved with a Fab fragment having a heavy chain sequence of SEQ ID NO: 48 and a light chain sequence of SEQ ID NO: 49 in the otherwise identical immunoassay setup.
9. A polynucleotide encoding
   (i) the heavy chain or heavy chain variable domain of the monoclonal antibody according to any one of clauses 1 to 8, and/or
   (ii) the light chain or light chain variable domain of the monoclonal antibody according to any one of clauses 1 to 8.
10. A vector comprising the polynucleotide according to clause 9.
11. A host cell comprising the polynucleotide according to clause 9, or the vector according to clause 10.
12. A method of producing the monoclonal antibody according to any one of clauses 1 to 8, said method comprising culturing the host cell according to clause 11 and isolating said antibody.
13. A composition comprising the antibody according to any one of clauses 1 to 8, the polynucleotide according to clause 9, the vector according to clause 10, and/or the host cell according to clause 11.
14. Use of the antibody according to any one of clauses 1 to 8 or the composition according to clause 13 for in vitro detection or quantification of T4, in particular free T4 in a sample.
15. An in vitro immunoassay method for quantifying T4, in particular free T4 in a sample using the antibody as defined in any one of clauses 1 to 8.
16. A kit comprising the antibody as defined in any one of clauses 1 to 8 or the composition as defined in clause 13.

### DESCRIPTION OF THE FIGURES

The following figures are provided to aid the understanding of the present invention, the true scope of which is set forth in the appended claims. It is understood that modifications can be made in the procedures set forth without departing from the spirit of the invention.

FIG. 1 shows the data obtained for the initial kinetic screening of the generated antibodies. The dissociation rate constants k_{d} were plotted vs. the association rate constants kₐ, the diagonals indicate the resulting affinity-ranges.

Depicted are the 840 analyzed supernatants (A) and the chosen Ab-selection (B) of 50 antibodies. Importantly, no correction for mass transport limitation was applied for determination of the kₐ and k_{d} values depicted in this Figure, even though such correction would have been required.

FIG. 2 depicts the kinetic profiles for Fab fragments 38F8, 7D4, 7E10, 3B7, 18B3 and 4H8 binding L-T4 at 37° C. as measured by Biacore. A) 38F8, B) 7D4, C) 7E10, D) 3B7, E) 18B3 and F) 4H8 binding series of increasing L-T4 concentrations c=0.04-10 nM, dilution factor 3; duplicates for 3.3 nM. Multi cycle kinetics with measured sensorgrams (depicted black) overlayed with Langmuir 1:1 binding model with correction for mass transport limitations (8K-insight SW).

FIG. 3 shows the Affinity in solution (AiS) curves for Fab fragment. From left to right depicted are 38F8, 7D4, 7E10 (top) 3B7, 18B3 and 4H8 (bottom), concentration held constant at 3 nM (38F8, 7D4 and 7E10) resp. 5 (3B7) or 10 nM (18B3 and 4H8) and varying L-T4 concentrations. With increasing L-T4 present, the ‘free’ Fab fragment in solution decreases. The determined Fab concentrations for the competition experiment plotted versus the L-T4 competitor concentration.

FIG. 4 shows the kinetic profiles for Fab fragments 38F8, 7D4, 7E10, 3B7, 18B3 and 4H8 binding L-T3 at 37° C.: A) 38F8, B) 7D4, C) 7E10, D) 3B7, E) 18B3 and F) 4H8 binding series of increasing L-T3 concentrations c=1.2-300 nM, dilution factor 3; duplicates for 100 nM. Steady State kinetics with measured sensorgrams (left) and the plotted response reaching equilibrium in Resonance Units (RU) versus the XR concentration, overlaid with fitted binding model (right).

FIG. 5 shows a two-dimensional representation of the L-T4 ligand in the receptor binding pocket of Fab 38F8. The ligand is shown in stick representation while the receptor amino acids involved in the binding are simplified to spheres. Here the A and B letter prefixes represent the heavy and the light chains, respectively. Curved dotted lines that wrap around the ligand represent the main areas of interaction with the ligand while the straight dotted lines represent hydrogen bonds. The shaded spherical areas behind some of the ligand atoms show solvent accessibility, the larger the shaded sphere the more relative solvent accessibility.

FIG. 6 shows a sequence alignment of the VH domains and VL domains of 38F8, 7D4, 7E10, 3B7, 18B3 and 4H8, respectively. CDRs are highlighted in bold. The amino acid residues identified as part of the paratope which directly interacts with L-T4 according to the crystal structure of 38F8 in complex with L-T4 are underlined.

FIG. 7. Equilibrated 3D structures of 38F8, 7D4, 4H8, and 7E10. Sequences of CDRs of the heavy and light chains for all four clones are shown at the bottom. Amino acids forming the binding pocket of 38F8 and identical in other clones are shown in bold. Amino acids that are different in the binding pocket are shown in italics.

Description of Sequences
Amino acids (AS) 54 to 65 of the VH of 38F8
according to Kabat numbering
SEQ ID NO: 1:
SGNTYYASWAKG
AS 54 to 65 of the VH of 7D4 according to
Kabat numbering
SEQ ID NO: 2
SGNTYYATWAKG
AS 54 to 65 of the VH of 7E10 according to
Kabat numbering
SEQ ID NO: 3
SGSTYYATWAKG
AS 50 to 56 of the VL of 38F8 according to
Kabat numbering
SEQ ID NO: 4
GASTLTS
AS 50 to 56 of the VL of 7E10 and 7D4
according to Kabat numbering
SEQ ID NO: 5
GASTLAS
CDR-H1 sequence of 38F8 with variations in
the paratope residues
SEQ ID NO: 6
SNXMN, wherein X is V or A
CDR-H2 sequence of 38F8
SEQ ID NO: 7
YIWTRSGNTYYASWAKG
CDR-H3 sequence of 38F8 with variations in
the paratope residues
SEQ ID NO: 8
GLHIXYNIFNF, wherein X is G, A or V
CDR-L1 sequence of 38F8 with variations in
the paratope residues
SEQ ID NO: 9
QSSQSVX1X2NAWCS, wherein X1 is H or Y and X2 is N
or K
CDR-L3 sequence of 38F8 with variations in
the paratope residues
SEQ ID NO: 10
AGX1X2X3GSTX4X5HV, wherein X1 is G or A, X2 is Y,
W or F, X3 is S or T, X4 is Y or F, X5 is N, S, T,
Q, N
CDR-L2 sequence of 38F8
SEQ ID NO: 11
GASTLTS
SEQ ID NO: 12
CDR-H1 sequence of 38F8
SNVMN
CDR-H3 sequence of 38F8 with variations in
the paratope residues
SEQ ID NO: 13
GLHIXYNIFNF, wherein X is G or A
CDR-H3 sequence of 38F8
SEQ ID NO: 14
GLHIGYNIFNF
CDR-L1 sequence of 38F8
SEQ ID NO: 15
QSSQSVHNNAWCS
CDR-L3 sequence of 38F8 with variations in
the paratope residues
SEQ ID NO: 16
AGX1X2X3GSTX4X5HV, wherein X1 is G or A; X2 is Y,
W or F; X3 is S or T, X4 is Y or F, X5 is N, S, T,
CDR-L3 sequence of 38F8 with variations in
the paratope residues
SEQ ID NO: 17
AGGX1SGSTYX2HV, wherein X1 is Y, W and X2 is N, S
CDR-L3 sequence of 38F8
SEQ ID NO: 18
AGGYSGSTYNHV
CDR-H1 consensus sequence of 38F8, 7E10 and
7D4
SEQ ID NO: 19
X1NVX2N, wherein X1 is S or R and X2 is M, L
CDR-H2 consensus sequence of 38F8, 7E10 and
7D4
SEQ ID NO: 20
YIWTX1SGX2TYYAX3WAKG, wherein X1 is R, G or D; X2
is N or S and X3 is S or T
CDR-H3 consensus sequence of 38F8, 7E10 and 7D4
SEQ ID NO: 21
GLX1IGYX2IFNF, wherein X1 is H or A and X2 is N or
A
CDR-L1 consensus sequence of 38F8, 7E10 and
7D4
SEQ ID NO: 22
QSSQSVX1X2NX3WX4S, wherein X1 is H or Y and X2 is
N or K, X3 is A or N and X4 is Cor L
CDR-L3 consensus sequence of 38F8, 7E10 and
7D4
SEQ ID NO: 23
AGGX1SX2X3X4YX5HX6, wherein X1 is Y or W; X2 is G,
A or S, X3 is S, G or N and X4 is T. G or S, X5 is
N or S and X6 is V or A
CDR-L2 consensus sequence of 38F8, 7E10 and
7D4
SEQ ID NO: 24
GASTLX1S, wherein X1 is T or A
CDR-H1 of 7E10
SEQ ID NO: 25
RNVMN
CDR-H1 of 7D4
SEQ ID NO: 26
RNVLN
CDR-H2 of 7E10
SEQ ID NO: 27
YIWTDSGSTYYATWAKG
CDR-H2 of 7D4
SEQ ID NO: 28
YIWTGSGNTYYATWAKQ
CDR-H3 of 7D4 and 7E10
SEQ ID NO: 29
GLAIGYAIFNF
CDR-L1 of 7D4 and 7E10
SEQ ID NO: 30
QSSQSVYKNNWLS
CDR-L3 of 7E10
SEQ ID NO: 31
AGGWSSNSYNHA
CDR-L3 of 7D4
SEQ ID NO: 32
AGGWSAGGYSHA
CDR-L2 of 7D4 and 7E10
SEQ ID NO: 33
GASTLAS
FW-H1 of 38F8 according to Kabat
SEQ ID NO: 34
LSLEESGGRLVTPGTPLTLTCTVSGIDLS
FW-H2 of 38F8 according to Kabat
SEQ ID NO: 35
WVRQAPGKGLEWIG
FW-H3 of 38F8 according to Kabat
SEQ ID NO: 36
RFTISKTSSTTVDLKMTSLTTEDTATYFCAG
FW-H4 of 38F8 according to Kabat
SEQ ID NO: 37
WGQGTLVTVSS
FW-L1 of 38F8 according to Kabat
SEQ ID NO: 38
AVLTQTPSPVSAAVGGTVTINC
FW-L2 of 38F8 according to Kabat
SEQ ID NO: 39
WFQKKPGQPPKQLIY
FW-L3 of 38F8 according to Kabat
SEQ ID NO: 40
GVPSRFKGSGSGTQFTLTISDVQCDDAATYYC
FW-L4 of 38F8 according to Kabat
SEQ ID NO: 41
FGGGTEVVVK
VH domain of 38F8
SEQ ID NO: 42
LSLEESGGRLVTPGTPLTLTCTVSGIDLSSNVMNWVRQAPGKGLEWIGYI
WTRSGNTYYASWAKGRFTISKTSSTTVDLKMTSLTTEDTATYFCAGGLHI
GYNIFNFWGQGTLVTVSS
VH domain of 7E10
SEQ ID NO: 43
QSVEESGGRLVTPGTPLTLTCTVSGIDLSRNVMNWVRQAPGKGLEWIGYI
WTDSGSTYYATWAKGRFTISKTSSTTVELKMTSPTTEDTATYFCAGGLAI
GYAIFNFWGQGTLVTVSS
VH domain of 7D4
SEQ ID NO: 44
QSVEESGGRLVTPGTPLTLTCTVSGIDLSRNVLNWVRQAPGKGLEWIGYI
WTGSGNTYYATWAKGRFTISKTSSTTVDLKMTSPTTEDTATYFCAGGLAI
GYAIFNFWGQGTLVTVSS
VL domain of 38F8
SEQ ID NO: 45
AVLTQTPSPVSAAVGGTVTINCQSSQSVHNNAWCSWFQKKPGQPPKQLIY
GASTLTSGVPSRFKGSGSGTQFTLTISDVQCDDAATYYCAGGYSGSTYNH
VFGGGTEVVVK
VL domain of 7E10
SEQ ID NO: 46
AVLTQTPSPVSAAVGGTVTISCQSSQSVYKNNWLSWFQQKPGQPPKLLIY
GASTLASGVPSRFEGSGSGTQFTLTISDVQCDDAATYYCAGGWSSNSYNH
AFGGGTGVVVT
VL domain of 7D4
SEQ ID NO: 47
AAVLTQTPSPVSAAVGGTVTISCQSSQSVYKNNWLSWFQQKPGQPPKLLI
YGASTLASGVPSRFEGSGSGTQFTLTISDVQCDDAATYYCAGGWSAGGYS
HAFGGGTGVVVA
Heavy chain of Fab 38F8
SEQ ID NO 48
LSLEESGGRLVTPGTPLTLTCTVSGIDLSSNVMNWVRQAPGKGLEWIGYI
WTRSGNTYYASWAKGRFTISKTSSTTVDLKMTSLTTEDTATYFCAGGLHI
GYNIFNFWGQGTLVTVSSGQPKAPSVFPLAPCCGDTPSSTVTLGCLVKGY
LPEPVTVTWNSGTLTNGVRTFPSVRQSSGLYSLSSVVSVTSSSQPVTCNV
AHPATNTKVDKTVAPSTCS
Light chain of Fab 38F8
SEQ ID NO 49
AVLTQTPSPVSAAVGGTVTINCQSSQSVHNNAWCSWFQKKPGQPPKQLIY
GASTLTSGVPSRFKGSGSGTQFTLTISDVQCDDAATYYCAGGYSGSTYNH
VFGGGTEVVVKGDPVAPTVLIFPPAADQVATGTVTIVCVANKYFPDVTVT
WEVDGTTQTTGIENSKTPQNSADCTYNLSSTLTLTSTQYNSHKEYTCKVT
QGTTSVVQSFNRGDC
VH domain of 18B3
SEQ ID NO: 50
QSVEESGGRLVTPGTPLTLTCTLSGFSLKGYALSWVRQAPGKGLEWIGLI
GNTGMTYYATWATGRFTISKTSTTVDLKMTSPTTEDTATYFCARDWFRYD
TFGGTTVIYYYGMDLWGPGTLVTVSS
VH domain of 4H8
SEQ ID NO: 51
QSVEESGGRLVTPGTPLTLTCTASGESLSAYYMIWVRQAPGKGLEWIGYI
GGGVSASYASWANGRFTISSTSTTVDLKIPSPTTEDTATYFCARGSWNSG
IDLWGQGTLVTVSS
VH domain of 3B7
SEQ ID NO: 52
QSLEESGGDLVKPGASLTLTCKASGIDFSGSALCWVRQAPGKGPEWIVCI
YVGSFQNTYYASWAKGRFTISKTSSTTVTLQMTSLTVADTATYFCASDAS
GISHYRYYFNLWGPGTLVTVSS
VL domain of 18B3
SEQ ID NO: 53
ALVMTQTPSPVSAAVGGTVTINCQASEEIGNNLAWFQQKPGQPPKLLIQR
ASTLASGVPSRFSGSGSGTDYSLTISGLQCDDAATYYCLGVLPYIGADGH
AFGGGTEVVVKGDPV
VL domain of 4H8
SEQ ID NO: 54
AAVLTQTASPVSAAVGGTVTINCQSSQSVVNNNRLSWFQQKPGQPPKLLI
YKASTLASGVPSRFKGSGSGTQFTLTISDVQCDDAATYYCLGGYISTSDN
AFGGGTEVVVKGDPV
VL domain of 3B7
SEQ ID NO: 55
AIEMTQTPFSVSAAVGGTVTISCQASESVYAKLGWYQQKPGQPPKLLIYD
ASSLASGVPSRFKGSGSGTEYSLTISDLECDDAATYYCQSAYYTRGADTW
GAFGGGTEVVVKGDPV
CDR-H1 of 4H8
SEQ ID NO: 56
AYYMI
CDR-H2 of 4H8
SEQ ID NO: 57
YIGGGVSASYASWANG
CDR-H3 of 4H8
SEQ ID NO: 58
GSWNSGIDL
CDR-L1 of 4H8
SEQ ID NO: 59
QSSQSVVNNNRLS
CDR-L2 of 4H8
SEQ ID NO: 60
KASTLAS
CDR-L3 of 4H8
SEQ ID NO: 61
LGGYISTSDNA

### EXAMPLES

The following examples are provided to aid the understanding of the present invention, the true scope of which is set forth in the appended claims. It is understood that modifications can be made in the procedures set forth without departing from the spirit of the invention.

### Example 1: Generation of Antibodies Specific for T4

### Synthesis of Hapten Immunogens and Screening Reagents

### Synthesis of Hapten T4-NH-PEG(3)-(OSu)

100 mg of L-Thyroxin was dissolved in 8 mL of dry DMF and 38 μL of trimethylamine and 117 mg of NHS-PEG3-NHS were added. The reaction was stirred for 4 h. The solvent was evaporated and the product was purified by prep. HPLC reverse phase.

HPLC-ESI-MS: M+=1107.2 Da. The yield was 105 mg

### Synthesis of the Antigen Conjugate T4-NH-PEG(3)-CO-KLH

7.9 mg of T4-NH-PEG(3)-(OSu), NHS ester synthesized as described above, was dissolved in 1500 μL DMSO and added to a solution of 100 mg KLH (Keyhole Limpet Hemocyanin, Sigma H 8283). The pH was adjusted to pH=8.3 and the solution stirred overnight. The mixture was purified in an Amicon stirred cell.

Analytics of amino groups: Antigen-KLH ratio—ca. 200:1

The chemical structure of T4-NH-PEG(3)-CO-KLH is as follows:

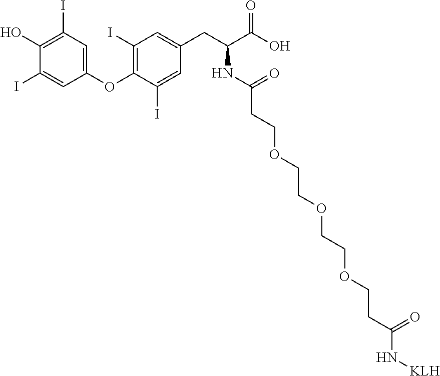

### Synthesis of T4(OSu)-bis-DADOO-Biotin

Biotin-DADOO-HS-DADOO (121 mg, see EP451810A1) was dissolved in DMF (20 mL) with triethylamine (36.1 μL) and T4-(Boc)-OSu (249 mg) was added. The reaction was stirred for 2 h. The solvent was removed on a rotary evaporator. The crude product was dissolved in TFA (6.0 mL) and stirred for 30-60 min at room temperature. The solvent was evaporated and the product was purified by prep. HPLC reverse phase to give 147 mg.

HPLC-EST-MS: [M+2H⁺]/2=682.9 Da. The yield was 147 mg.

The chemical structure of T4(OSu)-bis-DADOO-Biotin is as follows:

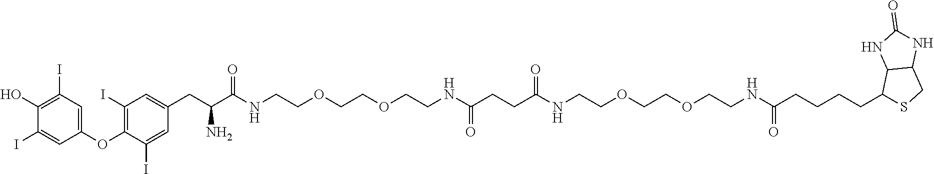 [SMILES: N[C@@H](Cc1cc(I)c(Oc2cc(I)c(O)c(I)c2)c(I)c1)C(=O)NCCOCCOCCNC(=O)CCC(=O)NCCOCCOCCNC(=O)CCCCC1SCC2NC(=O)NC21]

### Immunization

New Zealand White (NZW) rabbits, 12-16 weeks old, were immunized with T4-NH-PEG(3)-CO-KLH. To enhance the immunogenicity of the hapten it was coupled to keyhole limpet hemocyanin (KLH) as a carrier protein. In the first month the animals were immunized weekly. Starting in the second month, the immunization schedule was reduced to once per month. For the first immunization 500 pg T4-NH-PEG(3)-CO-KLH was dissolved in 0.9% NaCl and emulsified in 2 ml complete Freund's Adjuvant (CFA). For all following immunizations, CFA was replaced by 1 mL Incomplete Freund's Adjuvant (IFA) emulsion.

### Titer Analysis

Titer analysis was performed with an ELISA protocol. Serum titrations were performed using T4(OSu)-bis-DADOO-Biotin as a positive control.

Biotinylated screening reagents were immobilized on the surface of 96 well streptavidin-coated microtitre plates by incubating 100 μl per well of a 16 ng/ml solution for 60 min at room temperature. Subsequent washing was performed using an automated instrument (Biotek) according to manufacturer's instructions. A small amount of serum from each rabbit (2-3 ml per animal) was collected on day 45 and day 105 after the start of the immunization campaign. The serum from each rabbit was diluted 1:300, 1:900, 1:2700, 1:8100, 1:24300, 1:72900, 1:218700 and 1:656100 with PBS containing 1% BSA. 100 μl of each dilution was added to the plate previously prepared with the screening peptides and incubated for 60 min at room temperature. Bound antibody was detected with a HRP-labeled F(ab′)2 goat anti-rabbit Fcγ (Dianova) and ABTS substrate solution (Roche). The titer of the analyzed animals was set at 50% signal decrease of the dilution curve.

TABLE 1
Exemplary titers after immunization with
T4-NH-PEG(3)-CO-KLHT4-CO-PEG(3)-CO-KLH

| animal | titer day 45 | titer day 105 |
|---|---|---|
| 1#K6212 | 340.102 | 161.126 |
| 2#K6213 | 186.370 | 260.575 |
| 3#K6214 | 157.993 | 234.107 |
| 4#K6216 | 360.621 | 321.341 |

As demonstrated by the results of Table 1, the polyclonal sera from immunized animals bound to the T4(OSu)-bis-DADOO-Biotin screening peptide.

### B-Cell Cloning

For enrichment of antigen reactive B-cells, 100 ng/ml T4(OSu)-bis-DADOO-Biotin was pre-incubated with the peripheral blood mononuclear cell (PBMC) pool from the immunized animals for 15 min at 4° C. After a washing step, the antigen-reactive B cells bound to the T4(OSu)-bis-DADOO-Biotin were incubated with streptavidin-coated beads (Miltenyi) for 15 min at 4° C. Sorting of positive B-cells using MACS columns (Miltenyi) and subsequent incubation were performed as described in Seeber et al., PLoS One 9(2014), issue 2, e86184, with the only exception that the sorting of positive B cells involved MACS columns (Miltenyi) instead of plate binding.

Subsequently Hit-ELISA (i.e. ELISAs testing the binding to the screening agents) was used to identify B-cells expressing antibodies having desired binding characteristics, i.e. binding the T4(OSu)-bis-DADOO-Biotin. T4(OSu)-bis-DADOO-Biotin was immobilized on the surface of streptavidin-coated 96-well plates (Nunc) by incubation of 100 μl per well of 100 ng/ml solutions for 60 min at room temperature, respectively. The plates were washed and 30 μl of rabbit B-cell culture supernatant was transferred to each well and incubated for 1h at room temperature. For the detection of antibodies bound to the screening agents, HRP-labeled F(ab′)2 goat anti-rabbit Fcγ (Dianova) and ABTS substrate solution (Roche) were used according to manufacturer's instructions. 5868 clones were identified that bound to T4(OSu)-bis-DADOO-Biotin. 5868 clones were identified that bound to T4(OSu)-bis-DADOO-Biotin (out of ten B cell sorting experiments with in total five immunized rabbits). The V regions of 333 clones from a first B cell sort and 421 clones of a second B cell sort were cloned into mammalian expression vectors and subsequently expressed in 2 ml of HEK293 cells (described in Seeber et al., PLoS One 9(2014), issue 2, e86184.). After one week of expression the supernatants of the transfected HEK293 cells, containing rabbit IgG, were then used for an initial SPR Biacore based selection of a subset of antibodies fulfilling performance criteria for detailed kinetic analysis (see Examples 2 and 3) and evaluation in an Elecsys® platform based fT4 assay (see Example 4).

### Example 2: SPR Biacore Kinetic Screening to Select Antibodies

The 840 recombinantly produced monoclonal antibodies identified to bind T4(OSu)-bis-DADOO-Biotin by ELISA in Example 1 were subjected to a further screening step using SPR Biacore. Specifically, 50 antibodies were preselected according to kinetic features and finally a set of six antibodies was selected. In the course of the selection and a following detailed assessment of the six selected antibodies, the following characteristics of the antibodies were assessed: kinetic parameters (K_{D}, kₐ, k_{d}, velocity factor) for binding L-T4 and potential cross reactivity with L-T3, D-T3, rT3, 3,3′,5-triiodothyroacetic acid, 3,3′,5,5′-tetraiodothyroacetic acid, 3,5-diiodo-L-tyrosine and 3-iodo-L-tyrosine.

The kinetic screening was performed at 37° C. on a GE Healthcare BIAcore™ T200 instrument. A Biacore CM5 Series S sensor was mounted to the instrument and was preconditioned according to the manufacturer's instructions.

The system buffer was PBS, pH 7.4 containing 11 mM P04, 137 mM NaCl, 2.7 mM KCl, pH 7.4+0.05% (w/v) Tween20 and 5% (v/v)DMSO.

The system buffer, supplemented with 1 mg/mL Carboxymethyldextran (CMD) was used as sample buffer.

A rabbit antibody capture system was immobilized on the sensor surface. The system buffer was HBS-ET+pH 7.4, containing 10 mM HEPES, pH 7.4, 150 mM NaCl, 3 mM EDTA, 0.05% (w/v) Tween20. A polyclonal goat anti-rabbit IgG Fc capture antibody GARbFcγ (Code-No. 111-005-046, Jackson Immuno Research) was amine coupled using the EDC/NHS-chemistry according to the to the manufacturer's instructions at 25° C.

25 μg/mL capture antibody were used in 10 mM sodium acetate buffer pH 5.0. Capturing antibodies were immobilized with ligand densities between 13000-15000 RU on flow cells Fc1-4. Free activated carboxyl groups were saturated with 1 M ethanolamine pH 8.5.

25 nM rabbit normal IgG (SIGMA) were used as reference on FC1. Rabbit antibody containing cell culture supernatants were diluted in sample buffer and were captured on flow cells 2, 3 and 4. Capturing was performed at 5 μl/min for 3 minutes. The antibody Capture Level (CL) were monitored.

The kinetic screening of 840 cell culture supernatants was performed with single concentration injections of 30 nM L-T4 (Roche) analyte. The analyte association was monitored for 3 minutes, the dissociation phase for 5 minutes at 60 μl/min. After each measurement cycle the capture systems were regenerated by injections of 10 mM Glycine buffer pH 2.0 and pH 2.25 at 20 μL/min for 30 and 60 seconds.

Two report points, the recorded signal shortly before the end of the analyte injection, Analyte Binding Late (BL) and the signal shortly before the end of the dissociation time, Stability Late (SL), were used to characterize the antibody/antigen binding stability.

The association rate constant kₐ [M⁻¹s⁻¹], the dissociation rate constant k_{d} [s⁻¹] and the dissociation equilibrium constant K_{D} [M] were calculated according to a Langmuir model using the evaluation software. The antibody/antigen complex half-life was calculated in minutes according to the formula t/2 diss=ln(2)/(k_{d}*60). Importantly, in this initial screening of numerous antibodies no mass transport correction was applied, as no correction for MTL is offered in the used T200 Evaluation-Software. Thus, in the event that a binding between an antibody and the L-T4 is diffusion limited in the Biacore setting the kₐ rates may be underestimated. Notably, in Example 3, MTL correction was applied.

The Molar Ratio, the binding stoichiometry was calculated by the formula

MR=BL(antigen)*MW(antibody)/(MW(antigen)*CL (antibody)).

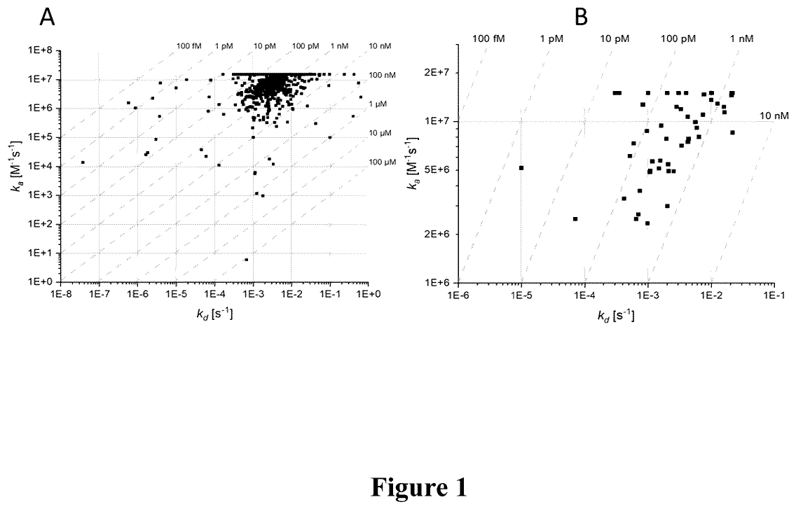

In this way 840 rabbit mAbs were kinetically investigated. The kinetic data of the antibody pool was statistically evaluated. The mean value of the entire dissociation equilibrium constants K_{D} was 7×10⁻¹⁰ M. The highest affinity found was K_{D} 1.94×10⁻¹² M and the lowest affinity in the antibody pool was of K_{D} 1.44×10⁻⁹ (see FIG. 1). The 840 antibody kinetics were first ranked according to their dissociation rate constants and a set of 50 antibodies were empirically chosen from different complex stability corridors populating around the highest, the lowest and the mean T4 complex stabilities (see FIG. 1). These 50 recombinant antibodies were then kinetically investigated in more detail by multi cycle kinetics with T4 analyte concentrations between 0.04 nM and 30 nM at two different temperatures 13° C. and 37° C. Again, no mass transport correction was applied, even if the measured signature would have indicated such correction. The assay settings from the kinetic screening were used as described. The goal was to identify suitable antibodies using the Velocity Factor principle (EP 2470563B1). The velocity factor as used herein corresponds to the quotient kₐ (37° C.)/kₐ (13° C.). Antibodies with already fast L-T4 association rate constants kₐ [M⁻¹s⁻¹] at low temperature 13° C. and not much accelerating association rate constants kₐ at elevated temperature 37° C. were in the focus. Antibodies with a dramatic increase of kₐ in the temperature gradient from 13° C. to 37° C. possess an unwanted high binding entropy and were deselected.

E.g., the antibody 3B7 shows a quotient kₐ (37° C.)/kₐ (13° C.)=18 and was selected, whereas the antibody 18C5 shows a quotient kₐ 37° C./kₐ 13° C.=152 and was deselected.

From this association rate constant focused evaluation 6 mAbs were selected and subjected to more detailed kinetically characterized for binding to L-T4 and potential cross reactants 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and 3-i-L-Thyrosine.

L-T3, D-T3, rT3, 3,3′,5-triiodothyroacetic acid, 3,3′,5,5′-tetraiodothyroacetic acid, 3,5-diiodo-L-tyrosine and 3-iodo-L-tyrosine.

The selected antibodies were 38F8, 7D4, 7E10, 3B7, 18B3 and 4H8. These selected antibodies had a velocity factor of 2 to 35 (without applying MTL correction).

### Example 3: Further SPR Biacore Characterization of the 6 Antibodies Selected in Example 2

In Example 2, six antibodies have been selected for further detailed assessment. For this assessment not the original IgGs but corresponding Fab fragments thereof were used. The Fab fragments w ere recombinantely expressed and purified as described in Example 4A) below.

### Detailed Kinetic Characterization of the 6 Selected Antibodies

Affinities for 6 surface displayed Fab fragments were determined with a GE Healthcare Biacore™ 8K instrument at 37° C. The measurements were performed as described above with following adaption.

As capture system PAK<K-F(ab)2>Z-IgG(IS) (Code-No. 111-005-006, Jackson Immuno Research) was used. Amine coupling was performed as described using a HBS-N buffer pH 7.4 with a concentration of 35 μg/mL PAK<K-F(ab)2>Z-IgG(IS) in 10 mM sodium acetate buffer pH 5.0.

The multi cycle kinetics was performed using a series of increasing L-T4 concentrations c=0.12-10 nM, dilution factor 3. Buffers used as described in Example 2. Regeneration was performed by injections of 10 mM Glycine buffer pH 2.0 and pH 2.25 at 20 μL/min for 60 seconds.

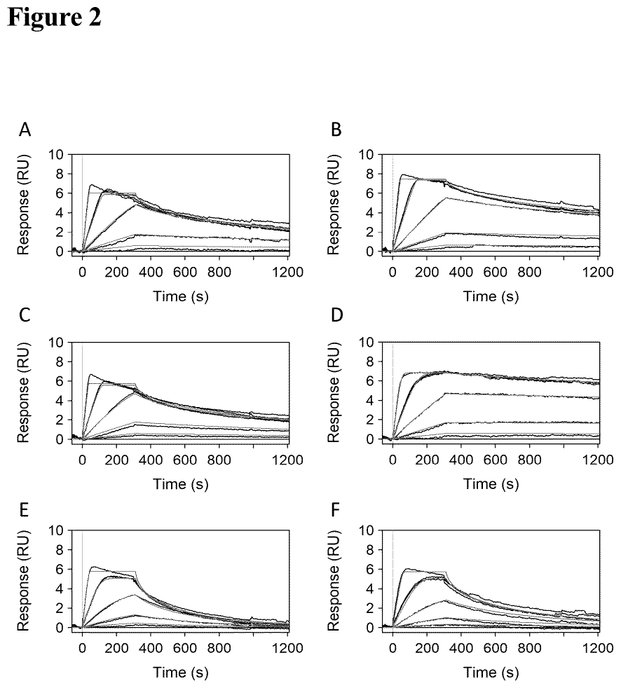

Interactions at 37° C. are shown in FIG. 2.

Interactions for Fabs 38F8, 7D4 and 7E10 showed massive mass transport limitation (MTL), when binding L-T4, whereas Fab 3B7 showed less MTL. MTL correction was applied automatically using the Evaluation Insight Software V3.011.15423 from the vendor. Here, the MTL correction is addressed using the 2-compartment model. Therefore, the kinetic constants represent apparent values, although corrected for MTL.

The Fabs 38F8, 7D4 and 7E10 showed significantly accelerated complex formation velocities compared to Fab 3B7, 18B3 or 4H8 at 37° C.;

The kₐ rate constants at 37° C. for 38F8, 7D4 and 7E10 are >1.0E+09 M⁻¹s⁻¹, which is close to or outside the instrument specification. The kₐ-rate constants for 3B7, 4H8 and 18B3 were between 4.8E+06 to 1.8E+07 M⁻¹s⁻¹.

Resulting affinities were determined by calculation: K_{D}=280 pM (38F8), 125 pM (7D4), 248 pM (7E10), 37 pM (3B7), 960 pM (18B3) and 1210 pM (4H8); see Table 2 below.

TABLE 2
Affinity for surface displayed Fab fragments 38F8, 7D4, 7E10,
3B7, 18B3 and 4H8 binding L-T4 in solution at 37° C.

| Fab clone | L-T4 K_{D} [nM] |
|---|---|
| 38F8 | 0.28 ± 0.7% |
| 7D4 | 0.13 ± 0.6% |
| 7E10 | 0.25 ± 0.6% |
| 3B7 | 0.37 ± 1.8% |
| 18B3 | 0.96 ± 0.4% |
| 4H8 | 1.21 ± 0.7% |

### Affinity in Solution at 37° C.

Complementary to the affinities obtained from the kinetic rate constants, the dissociation equilibrium constant K_{D} was determined via the Affinity in Solution (AiS). The advantage of affinity in solution analysis is that it does not underlie mass transportation limitation and that it allows measurement in equilibrium.

Biotinylated (Bi-) T4 was pre-captured on the CAP-chip sensor surface via Streptavidin (SA)-Biotin interaction. Mixtures of anti-T4-Fab-fragment and non-labeled T4 were pre-incubated for several hours for reaching equilibrium. The concentration of ‘free’ Fab fragments is determined via binding to the surface-displayed biotinylated T4 using a preceding Fab calibration for quantification.

While the Fab-fragment concentration is held constant in the mixtures, the T4 concentration was varied. With increasing T4 present, the ‘free’ Fab fragment in solution decreases.

The assay setup was as follows:

Following the vendor instructions for the CAP-Kit (Cytiva), subsequently to the CAP-Reagent the biotinylated T4 is reversibly captured on the sensor surface with high density. The regeneration was performed after each cycle using Guanidinium/NaOH solution.

Preincubation of both interaction partners in solution: Fab concentration, was kept constant at 10 nM for Fabs 18B3 and 4H8, for 3B7 5 nM resp. 3 nM for Fabs 38F8, 7E10 and 7D4. T4 Thyroid hormone concentration was individually optimized for each T4 interaction, i.e. c=0.07-150 nM (18B3 and 4H8), 0.07 nM-50 nM (3B7) 0.021 nM-90 nM (38F8, 7E10 and 7D4). The Affinity in Solution model from Biacore Evaluation software was used to evaluate the data.

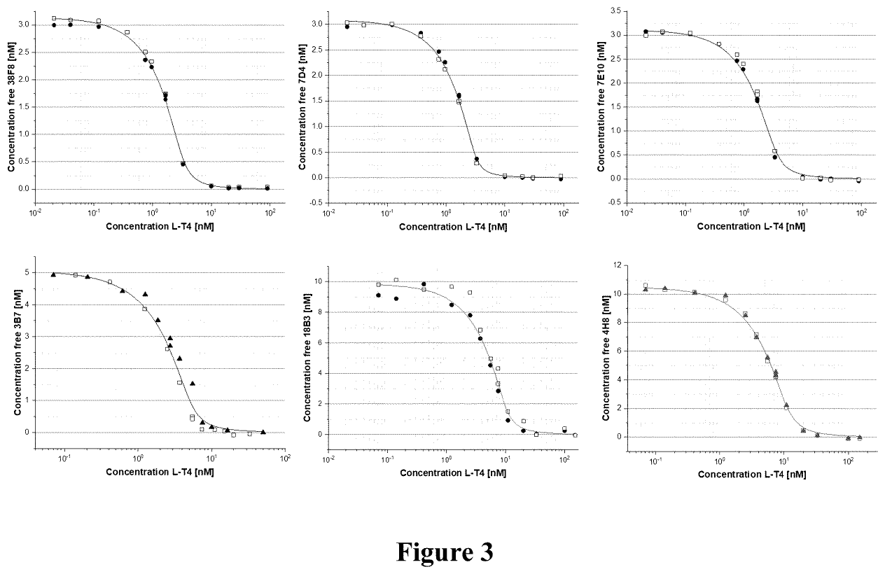

The Affinity in solution curves for Fab fragments 38F8, 7D4, 7E10, 3B7, 18B3 and 4H8 are shown in FIG. 3.

The K_{D} values determined with affinity in solution are as follows:

TABLE 3
Affinity in solution for Fab fragments
38F8, 7D4, 7E10, 3B7, 18B3 and 4H8

| Fab | AiS based K_{D} | | Bₜₒₜ |
|---|---|---|---|
| clone | [nM] | R² | [nM] |
| 38F8 | 0.16 ± 0.03 | 1.00 | 3.14 ± 0.02 |
| 7D4 | 0.08 ± 0.02 | 1.00 | 3.09 ± 0.02 |
| 7E10 | 0.21 ± 0.04 | 0.99 | 3.11 ± 0.03 |
| 3B7 | 0.24 ± 0.07 | 0.98 | 5.06 ± 0.07 |
| 18B3 | 0.44 ± 0.15 | 0.98 | 9.9 ± 0.1 |
| 4H8 | 0.70 ± 0.10 | 1.00 | 10.5 ± 0.1 |

The measured K_{D} values for clones 38F8, 7D4, 7E10 and 3B7 are in a similar range as measured with a different setting in the kinetic analysis) above and confirm these results.

Fab fragment 4H8 shows factor 1.7 higher affinity than measured via the kinetic analysis. Both, Fab fragments 18B3 and 4H8 show the weakest affinities of the six Fab fragments and confirm the ranking.

### Cross Reactivity

For the multi cycle cross reactivity measurements analyte concentrations from 0.1 nM to 900 nM were used. Potential cross reactants were injected between 30 μL/min to 60 μL/min. The association phases were monitored between 3 min to 5 min, the dissociation phases between 5 min to 15 minutes. The genuine T4 interactions were characterized using an additional analyte injection with 30 min dissociation time.

Affinities for 6 Fab fragments binding cross reactants were determined at 37° C. using multi cycle kinetics with a concentration series. Cross reactant L-T3 shows at least 49 fold weaker affinity than L-T4 at 37° C., rT3 shows at least 18 fold weaker affinity than L-T4 at 37° C.

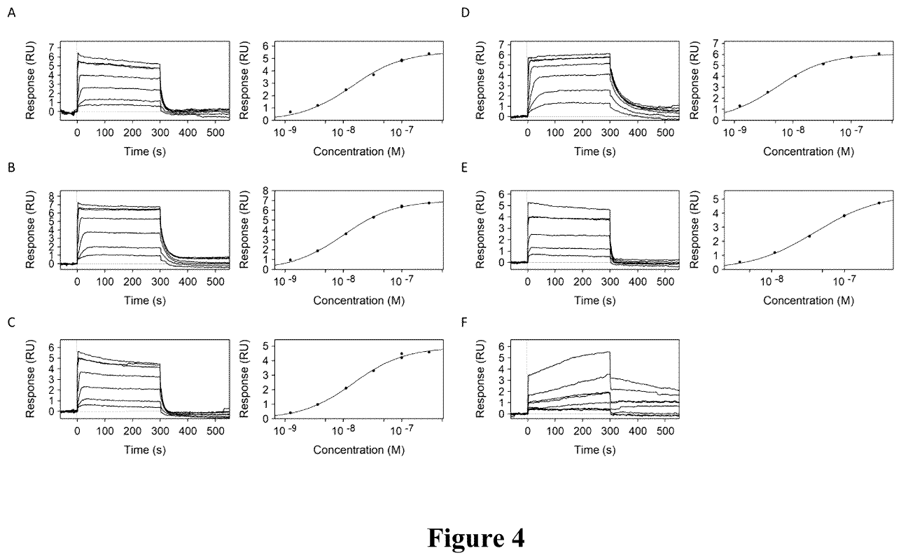

Interactions for cross reactant L-T3 at 37° C. are shown in FIG. 4. The results for all cross reactants are summarized in Table 4. For Fab 38F8 also additional cross reactants have been analyzed (see Table 5).

TABLE 4
Affinity Ratios K_{D} L-T3/K_{D} L-T4 resp. K_{D} rT3/K_{D} L-T4 for cross
reactants L-T3 and r-T3; n.b. means no binding detected

| Fab | Temp | Ratio | Ratio |
|---|---|---|---|
| clone | ° C. | K_{D} L-T3/L-T4 | K_{D} rT3/L-T4 |
| 38F8 | 25° C. | 47 | 14 |
| 38F8 | 37° C. | 62 | 20 |
| 7D4 | 25° C. | 52 | 10 |
| 7D4 | 37° C. | 58 | 18 |
| 7E10 | 25° C. | 80 | 31 |
| 7E10 | 37° C. | 91 | 35 |
| 3B7 | 25° C. | 99 | 152 |
| 3B7 | 37° C. | 164 | 190 |
| 18B3 | 25° C. | 50 | 30 |
| 18B3 | 37° C. | 49 | 32 |
| 4H8 | 25° C. | n.b. | 43 |
| 4H8 | 37° C. | n.b. | 27 |

TABLE 5
Affinity Ratios K_{D} for potential cross reactant
(XR)/K_{D} L-T4 for Fab clone 38F8

| | Ratio K_{D} XR/L-T4 |
|---|---|
| Cross reactant XR | [—] |
| 3-iodo-L-Thyrosine (L-T3) | 62 |
| rThyroid hormone (rT3) | 20 |
| 3,3′,5-tri-iodo-thyroacetic acid | 92 |
| 3,3′,5,5′-tetra-iodothyroacetic acid | 5 |
| 3,5-di-iodo-L-Thyrosine | 166 |
| 3-i-L-Thyrosine | no XR binding detectable |

### Example 4: Characterization of the Antibodies Selected in Example 2 in an Elecsys® Competitive Immunoassay

### A.) Purification of Anti-T4-Fabs

Antibody candidates preselected according to kinetic behaviour by BiaCore-Analysis (see Example 2 and 3) were expressed as His-tagged Fab-Fragments using transient transfection of HEK-cells. Culture supernatants were concentrated using a Vivaflow 200 ultrafiltration unit (Sartorius, Germany) with 10 kDa MW cutoff, with subsequent buffer exchange via dialysis or diafiltration against 20 mM KPO4, 150 mM NaCl, 10 mM Imidazol, pH 8.0. Subsequently, a Ni-NTA/IMAC affinity chromatography column (HisTrap, GE Healthcare, Sweden) was equilibrated with the dialysis buffer above and the conditioned supernatant was applied on the column at a flow-rate of 60 column volumes per hour. The Fab fragment was then eluted using a linear gradient of buffer A (20 mM KPO4, 150 mM NaCl, 10 mM Imidazol, pH 8.0) and buffer B (20 mM KPO₄, 150 mM NaCl, 500 mM Imidazol, pH 8.0) with 0-35% B in 10-20 column volumes. Fractions of eluting Fab were assessed with regards to purity using analytical size exclusion chromatography, and fractions with Fab of purity ≥95% were pooled. Finally, the pooled Fab-Preparation was dialyzed against a storage buffer of 20 mM KPO₄, 100 mM KCl, 2% Sucrose, pH 7.9, aliquoted and stored frozen at −80° C.

### B.) Generation of Anti-T4-Fab Ruthenium Label Conjugates

Purified anti-T4 Fab antibodies were labelled with a N-Hydroxy-succinimide activated ester of sulfo Bispiridyl-Ruthenium according to standard laboratory procedures. Shortly, to a solution of Fab (4.4 mg, 1.03 mL in 100 mM KPO₄ pH 8.4), 52 pL solution of SULFO-BPRU NHS ESTER (CAS 482618-42-8: Ruthenate(2-), bis[[2,2′-bipyridine]-4,4′-dimethanesulfonato(2-)-κN1,κN1′][1-[4-(4′-methyl[2,2′-bipyridin]-4-yl-κN1,κN1′)-1-oxobutoxy]-2,5-pyrrolidinedione]-, sodium (1:2), (OC-6-31)-(ACI); 12.5 mg/ml in DMSO) are added (i.e. stoichiometry of 6.0 mol Ruthenium-NHS-ester per mol Fab). After 120 min stirring at room temperature, the derivatization was stopped by addition of Lysine ad 10 mM final concentration. pH was adjusted to pH 7.5 with saturated KH₂PO₄, and the reaction mixture was dialysed against 50 mM KPO₄/0.15 M KCl/2% saccharose, pH 7.5 overnight. For removal of aggregates and remaining hydrolized Ruthenium ester, appropriate fractions were collected from Size-exclusion chromatography on an Superdex 75 HR 10/30 column (GE Healthcare Life Sciences). After addition of sucrose to a final concentration of 6.5% (w/v), preparations were filtered over 0.45 μm PVDF syringe filters (Acrodisk Pall Life Sciences Corp.) and stored frozen at −80° C.

### C.) Functional Assessment of Anti-T4 Fab-Ruthenium Conjugates

The assay principle of the competitive immunological assay using an automated Elecsys Immuno-Analyzer (Elecsys® Cobas® e411) is summarized as follows: The total incubation time required for the assay is 18 minutes: 1st incubation (9 min): 15 μL of a fT4 containing sample, 75 μL of ruthenylated monoclonal T4-specific Fab antibody are incubated and form a complex comprising a proportion of the ruthenylated anti T4 Fab antibody and the free T4. 2nd incubation (9 min): 75 μL of a solution of T4(OSu)-bis-DADOO-Biotin-Hapten-conjugate and 35 μL of Streptavidin-coated microparticles are added to the mixture of the first incubation step. During this second incubation the ruthenylated anti T4 Fab antibody which remains free after the first incubation step can bind to the biotinylated T4-Hapten, and the complex of ruthenylated antibody bound to the T4-Biotin conjugate binds to the Streptavidin coated magnetic bead/solid phase via interaction of biotin and streptavidin. The reaction mixture is aspirated into the measuring cell where the microparticles are magnetically captured onto the surface of the electrode. Unbound substances are then removed with ProCell #11662988122 (Roche Diagnostics GmbH Germany). Application of a voltage to the electrode then induces electro-chemiluminescence-based emission of light which is measured by a photomultiplier.

A plot of ECL signal in counts vs. concentration of free T4 results in a typical hyperbolical competition curve with falling signal with rising T4 concentration.

TABLE 6
Signal competition with rising concentrations of free T4 using
Ruthenium-conjugates of polyclonal anti-T4 antibodies (reference)
and various monoclonal antibodies acc. to the invention

| | Calibrator # | | | | | | |
|---|---|---|---|---|---|---|---|
| | MK1 | MK2 | MK3 | MK4 | MK5 | | Signal |
| | concentration of free T4 | | | | | total | Dynamics |
| | [pmol/L] | | | | | competition | [(cts MK1 - |
| | 0.00 | 9.53 | 25.69 | 52.71 | 122.10 | cts MK5/cts | cts MK5)/ |
| | Signal [cts] | | | | | MK1 [%] | cts MK5] |
| pAb lot A | 133262 | 33017 | 12327 | 6491 | 3803 | 3% | 34.0 |
| pAb lot B | 195660 | 65633 | 31877 | 18151 | 11101 | 6% | 16.6 |
| mAb 3B7 | 130543 | 67950 | 26980 | 14304 | 8432 | 6% | 14.5 |
| mAb 7D4 | 52230 | 30347 | 16196 | 9518 | 5863 | 11% | 7.9 |
| mAb 7E10 | 73167 | 27870 | 12531 | 6731 | 3991 | 5% | 17.3 |
| mAb 38F8 | 145082 | 40459 | 17338 | 9202 | 5272 | 4% | 26.5 |
| mAb 18B3 | 8032 | 7139 | 4959 | 3426 | 2331 | 29% | 2.4 |
| mAb 4H8 | 23559 | 22175 | 18720 | 14332 | 10231 | 43% | 1.3 |

As shown under Table 6 above, the three Fab fragments 38F8, 7E10 and 7D4, which showed an extraordinary high kₐ with respect to T4 in the Biacore experiments conducted in Examples 2 and 3 were among the 4 best antibodies tested. This confirms that a high kₐ with respect to T4 is one important factor (among others) to provide a competitive immunoassay. Furthermore, the data demonstrate that a polyclonal antibody can be successfully be replaced by single monoclonal antibodies as provided herein.

### Example 5: Determination of the Crystal Structure of Fab Antibody 38F8 in Complex with T4

Crystallization of Fab 38F8 with and without L-T4 Hormone:

A solution containing the 38F8 Fab fragment was concentrated to 18 mg/ml and subject to crystallization screening. Crystallization droplets were set up at 21° by mixing 100 nl of protein solution with 100 nl of reservoir solution (1:1 ratio), or 140 nl of protein solution with 60 nl of reservoir solution (7:3 ratio) in a vapor diffusion sitting drop experiment. Crystals appeared in several conditions containing polyethylene glycol (PEG) as a precipitating agent. Crystals used for structure determination appeared within two days and grew to full size within four days in a condition containing the following precipitant solution: 120 mM ethylene glycol mix (30 mM diethylene glycol, 30 mM triethylene glycol, 30 mM tetraethylene glycol and 30 mM pentaethylene glycol), 100 mM Tris base-BICINE pH 8.5, 20% v/v PEG 500 MME and 10% w/v PEG 20000.

Apo crystals were harvested from droplets with a ratio of 7:3 and crystals from droplets with a ratio of 1:1 were soaked for 20 hours in a saturated solution of L-thyroxine hormone (L-3,5,3′,5′-tetraiodothyronine or L-T4, Sigma), that contained 10% DMSO in addition to the precipitant solution described above. Crystals were harvested directly from the precipitant solution and flash-cooled in liquid N2. Diffraction images were collected with an EIGER2X 16M detector at a temperature of 100 K at the beam line X10SA of the Swiss Light Source and images were processed with the XDS package [Kabsch W. XDS. Acta Cryst. D66, 125-132 (2010)]. For the apo crystal, data from a single crystal were merged to yield a 1.81 Å resolution data set in space group C121 with two molecules in the asymmetric unit (see Table 7). For the L-T4 bound crystal, similarly, data from a single crystal were merged to yield a 1.66 Å resolution data set in space group C121 with two molecules in the asymmetric unit.

The structure was determined by molecular replacement with the program PHASER [McCoy A J, Grosse-Kunstleve R W, Adams P D, Winn M D, Storoni L C, Read R J J. Appl. Cryst. 40, 658-674 (2007)])] as a part of the PHENIX suite [Liebschner D. et al. Macromolecular structure determination using X-rays, neutrons and electrons: recent developments in Phenix. Acta Cryst. D75, 861-877 (2019)]. A Fab fragment from PDB-ID 6LDX was split into constant and variable domains and used as search models. The molecular replacement solution model was rebuilt in COOT [Emsley P, Lohkamp B, Scott W G, Cowtan K. Features and development of Coot. Acta Cryst. D66, 486-501 (2010)] and refined with PHENIX Refine.

TABLE 7
Crystallographic data and refinement statistics

| | Apo crystal | | L-T4₄ bound | |
|---|---|---|---|---|
| Data Collection | | | | |
| Wavelength (Å) | 1.0 | | 1.0 | |
| Resolution¹ (Å) | 53.3-1.81 | (1.93-1.81) | 38.98-1.66 | (1.67-1.66) |
| Spacegroup | C 1 2 1 | | C 1 2 1 | |
| Unit cell (Å, °) | 114.06, 85.90, 110.82 90.00, | | 114.27, 85.82. 111.58, 90.00, | |
| | 105.51, 90.00 | | 105.82, 90.00 | |
| Total reflections | 269,377 | (13,642) | 355,441 | (18,110) |
| Unique reflections | 77,732 | (3,887) | 103,427 | (5,172) |
| Multiplicity | 3.5 | (3.5) | 3.4 | (3.5) |
| Completeness (%) | 83.2 | (24.8) | 8.38 | (26.3) |
| Mean I/σ(I) | 12.9 | (1.5) | 12.7 | (1.4) |
| Wilson B-factor | 32.62 | | 27.86 | |
| R-meas | 0.049 | (0.610) | 0.046 | (0.950) |
| CC1/2 | 0.999 | (0.587) | 0.999 | (0.486) |
| Refinement | | | | |
| Reflections used in | 77,712 | (137) | 103,400 | (347) |
| refinement | | | | |
| Reflections used for R- | 3,831 | (5) | 5,254 | (14) |

| free | | |
|---|---|---|
| R-work | 17.49 | 17.68 |
| R-free | 21.90 | 21.49 |
| Number of non-hydrogen | 6.839 | 7.035 |
| atoms | | |
| macromolecules | 6.432 | 6.445 |
| Protein residues | 866 | 866 |
| RMS bonds (Å) | 0.005 | 0.007 |
| RMS angles (°) | 0.72 | 0.84 |
| Ramachandran favoured | 98.14 | 97.79 |
| (%) | | |
| Ramachandran allowed | 1.86 | 2.21 |
| (%) | | |
| Ramachandran outliers | 0.00 | 0.00 |
| (%) | | |
| Rotamer outliers (%) | 0.94 | 1.34 |
| Clashscore | 2.11 | 1.93 |
| Average B-factor (Å²) | 41.96 | 36.75 |
| macromolecules | 41.73 | 36.20 |
| ligands | 58.15 | 57.45 |
| solvent | 43.89 | 39.03 |

¹Values in parentheses refer to the highest resolution bins.
²Rₘₑₐₛ = Σ|I-<I>|/ΣI where I is intensity.
³ R_{work} = Σ|Fₒ-<F_{c}>|/ΣFₒ where Fₒ is the observed and F_{c} is the calculated structure factor amplitude.
₄R_{free} was calculated based on 5% of the total data omitted during refinement.

The Structure of Fab 38F8 with and without L-T4 Hormone

To characterize the binding of Fab 38F8 to L-T4 in atomic detail we determined the crystal structure of the Fab in the apo form and in the L-T4 ligand bound form. The overall conformation of the Fab is highly similar in both the apo and the ligand bound form with an RMSD of 0.11 A2. No significant differences are observed in side chains of the CDR-loops, suggesting a fast on-rate for ligand binding. This in line with the Biacore data shown in Example 3.

The Fab paratope is a pocket formed at the interface between the light and the heavy chain.

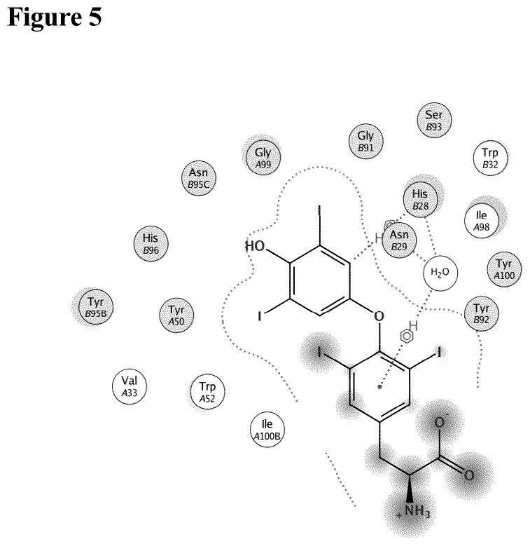

An analysis by the program PISA [Krissinel E and Henrick K. Inference of macromolecular assemblies from crystalline state. J. Mol. Biol. 372, 774-797 (2007)] revealed that a surface area of 416 A2 of the ligand is buried by Fab 38F8 which is 68% of the total ligand surface area. A total of sixteen amino acids constitute the Fab paratope, seven from the heavy chain and nine from the light chain (see FIG. 5 and Table 8). All CDR loops, apart from the light chain CDR2, participate in the ligand binding which is largely governed by hydrophobic interactions with the iodine atoms and the phenol rings. The hydrophilic amino and carboxyl groups of the ligand are exposed to solvent and point out of the pocket. The heavy and light chain CDR3 loops of the Fab form the bulk of the hydrophobic pocket, which is formed by both side chain and backbone interactions. These are summarized in Table 7.

Three hydrogen bonds help to stabilize the ligand that are formed by O4 and O4′ atoms of the ligand and Tyr50 on CRD2 of the heavy chain and His28 on CDR1 and His96 on CDR3 of the light chain of the fab. Additionally, two polar-pi (arene-H-bond) interactions are present where in one instance a water molecule, coordinated by His28 and Asn29 of the Fab light chain, is a hydrogen donor for one of the ligand phenol rings and in the other instance the C2′ is the hydrogen donor for the heterocyclic imidazole ring of His30 of the Fab light chain. All amino acid numberings are according to Kabat nomenclature.

TABLE 8
Summary of Fab 38F8 residue constituents of the paratope,
identified with a distance cutoff of 4.5 Å. Residues
are numbered according to the Kabat convention.

| | | Residue | | |
|---|---|---|---|---|
| | | according | | |
| | | to Kabat | Side | |
| Chain | CDR | numbering | chain/backbone | Interaction type |
| Heavy | 1 | Val33 | Side chain | Hydrophobic |
| Heavy | 2 | Tyr50 | Side chain | H-bond |
| Heavy | 2 | Trp52 | Side chain | Hydrophobic |
| Heavy | 3 | Ile98 | Side chain | Hydrophobic |
| Heavy | 3 | Gly99 | Backbone | Hydrophobic |
| Heavy | 3 | Tyr100 | Side chain | Hydrophobic |
| Heavy | 3 | Ile100b | Side chain | Hydrophobic |
| Light | 1 | His28 | Side chain | H-bond & H₂O |
| | | | | coordination |
| Light | 1 | Asn29 | Side chain | H₂O coordination |
| Light | 1 | Trp32 | Side chain | Hydrophobic |
| Light | 3 | Gly91 | Backbone | Hydrophobic |
| Light | 3 | Tyr92 | Backbone | Hydrophobic |
| Light | 3 | Ser93 | Backbone | Hydrophobic |
| Light | 3 | Tyr95b | Side chain | Hydrophobic |
| Light | 3 | Asn95c | Backbone | Hydrophobic |
| Light | 3 | His96 | Side chain | H-bond |

### Summary:

Fab 38F8 paratope is a pocket primarily made up of CRD3 loop residues from both the heavy and the light chains.
The conformational similarity between the apo and the ligand bound state of the Fab suggests a fast ligand binding on-rate.
Interactions with the L-T4 ligand are largely hydrophobic via the iodine atoms and the phenol rings but also includes three hydrogen bonds and two polar-pi interactions.
68% of the accessible surface area of the ligand is buried by the Fab

### Example 6: Sequence Comparison of Screened Antibodies

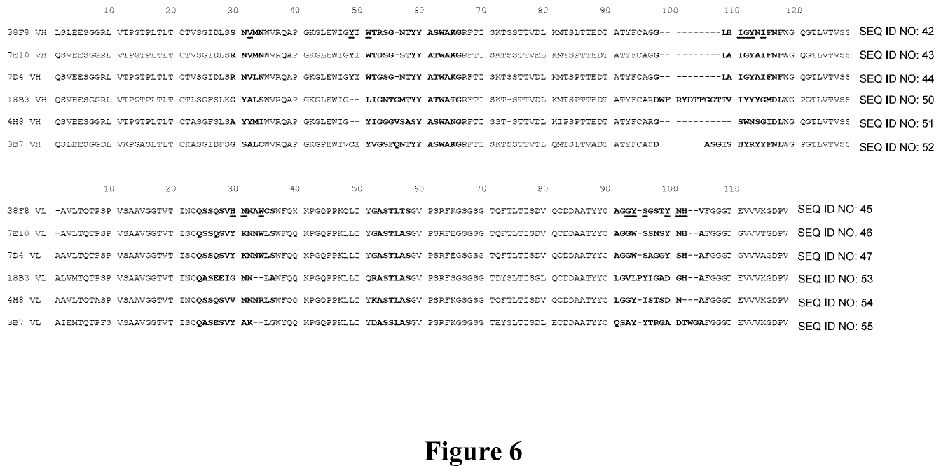

Example 3 revealed that Fab antibodies 38F8, 7E10 and 7D4 have an outstanding high kₐ. Moreover, Example 4 showed that these three clones are among the 4 best tested clones in an competitive immunoassay established on the Elecsys® system. To investige whether these three antibodies and some of the other six selected antibodies (see Example 2) share sequence similarities a sequence alignment of the variable regions (VH and VL) has been performed. The sequence alignment (see FIG. 6) surprisingly revealed that the antibodies 38F8, 7D4 and 7E10 have a strikingly high sequence similarity in the VH and VL region, in particular also in the CDRs. Even more strikingly, the conservation of the amino acid positions forming the paratope of the 38F8 antibody is extremely high. This suggests that 38F8, 7D4 and 7E10 are closely related antibodies sharing a high sequence similarity, especially also in the CDR sequences. As mentioned above, these antibodies share functional features, in particular an extremely high kₐ. Furthermore, these three Fab fragments were among the four best tested antibodies in an Elecsys® based immunoassay for quantifying fT4 as evaluated by signal to noise.

To quantify the observed similarity, a similarity score was obtained from a sequence alignment using a customized scoring matrix. This matrix is constructed based on five weighted physical parameters: shape index, van der Waals volume, isoelectric point, hydrophobicity, and polarizability. These five parameters capture most important characteristics of amino acids forming a paratope and allow to identify dissimilar clones with similar binding motives. Antibodies with a high similarity score have both similar binding mode as well as similar amino acid sequences in CDR regions. The higher the similarity score is, the more related the antibodies are. Similarity scores with respect to 38F8 for clones 7D4—98%, 7E10—98%, 3B7—86%, 18B3—92%, 4H8—95%. This similarity score analysis confirmed that 7E10 and 7D4 are closely related to 38F8. The paratope residues that mediate the binding are very similar (12 and 13 out of 16 amino acids are identical).

### Example 7: Modelling Analysis to Further Characterize the Binding of 38F8 and the Related Antibodies 7D4 and 7E10 to T4

The binding of Fab 38F8 to the L-T4 hormone on the atomic level was characterized using in silico approach. This analysis aimed to define which amino acids in the CDR are critical for the binding and to get evidence at which extent amino acid substitutions at specific CDR positions are acceptable.

The in silico analysis made use of the fact that with 7D4 and 7E10 two Fabs have been identified which are similar to 7E10 in their kinetic properties (kₐ and K_{D}) and only slightly worse in their Elecsys® performance. We also made use of the fact that 4H8 while sharing also a significant sequence similarity to 38F8 showed a huge difference in its kinetic characteristics as well as Elecsys® performance. Thus, the combination of known functional features and sequence variations could be used as basis for predicting the influence of amino acid substitutions on the functional characteristics (Kinetics, specificity and Elecsys® performance) of 38F8. In other words, this allowed us to identify critical amino acid residues in the CDRs (i.e. that cannot be substituted without loosing the functional performance), potentially critical amino acids (i.e. amino acids that can only be exchanged to certain amino acids) and non-critical amino acids (for which both conservative and non-conservative amino acids should be possible) for maintaining the 38F8 T4 binding characteristics (Kinetics, specificity and Elecsys® performance).

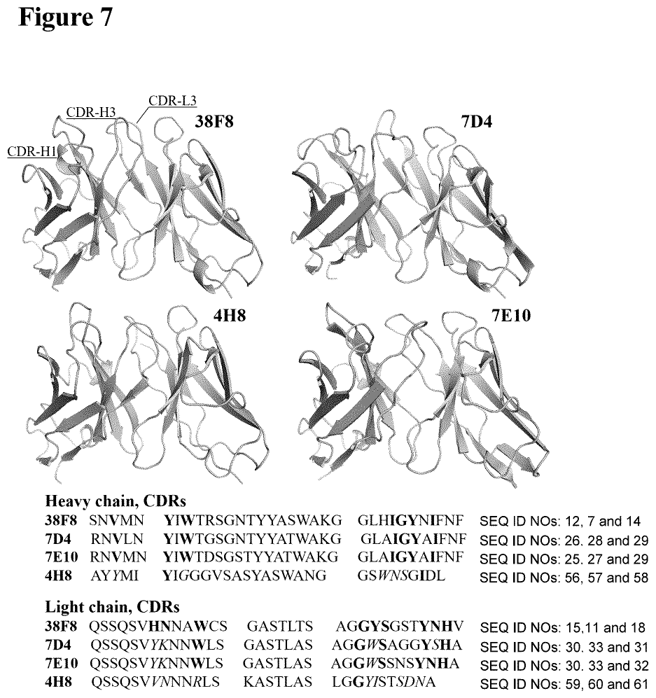

As an initial step, equilibrated structures of 38F8 and two highly similar antibodies, 7D4, 7E10 (with similarity scores of more than 98%) and 4H8 (with similarity score of 95%), were generated. To get equilibrated structures of 7D4, 7E10, and 4H8 we first predicted initial 3D shape using MoFvAB package based on machine learning algorithm trained on internal Roche antibody crystal structure database. After this initial guess, 3D structures were optimized further using molecular dynamic simulation in order to get correct loop shapes (details on simulation parameters are provided below). Crystal structure of 38F8 and equilibrated structures of 7D4, 7E10, and 4H8 are shown in FIG. 7. As a second step, we perform a point mutation in the sequence of 38F8, perform a free energy minimisation and compare with the initial structure and the structure or structure of three other clones.

The effect of point mutations in 38F8 on the binding with L-T4 hormone was modeled and predicted by comparing the sequences and 3D shapes of the highly similar clones with similar (7D4, 7E10) or different (4H8) functional characteristics (as shown in FIG. 7). All residues directly contributing to the paratope are listed in Example 5, above, and are printed in bold in the alignments in FIG. 7. Substitutions in the paratope region in the other three clones are shown in italics. With a few exceptions all of the residues of the paratope are considered as critical amino acids, i.e. amino acids that are crucial for maintaining the functional characteristics. However, some of the interactions in the paratope are only via the peptide backbone such that at these positions at least certain amino acid substitutions should be possible (see below). Moreover, other paratope amino acids make hydrophobic interactions with their side chain that can be mimicked by closely related amino acids (e.g. V33 of HC and Y95b of LC). These paratope amino acids that can be substituted to certain amino acids are grouped as potentially critical in Table 9. Non-critical regions listed in Table 9 are loop fragments that are far away from the binding pocket and conserve their structure even upon mutations in this region. Thus, the analysis proposes that amino acid exchanges in this “non-critical” regions should be possible. Such mutations can include conservative but likely also non-conservative amino acid exchanges. Moreover, potential critical amino acid positions in the CDRs were found, i.e. residues that do not contribute to the paratope but can likely be substituted only by certain other amino acids so as not to affect the structural position of critical paratope residues. Amino acids in the paratope region which contribute to binding only through backbone interactions or have a family variation in clones 7D4 and 7E10 are specified as potentially critical, and thus are excluded from the critical region. In the critical region only amino acids are included that are involved in the interaction through side chains and should not be exchanged. Table 9 summarizes the critical, potential critical and uncritical amino acids in the CDR regions of 38F8.

TABLE 9
Amino acids in CDR regions of 38F8, which are divided into three groups: non-critical (can be
substituted by other amino acids without loosing performance), potentially critical (can be
substituted by at least specific other amino acids without losing performance) and critical
(cannot be substituted). CDR loops are numbered according to the Kabat numbering scheme.

| REGION | CDR-H1 | CDR-H2 | CDR-H3 | CDR-L1 | CDR-L2 | CDR-L3 |
|---|---|---|---|---|---|---|
| NON- | S31, N32 | SGNTYYASWAKG | G95, L96, | QSSQS | GASTLTS | A89, G90 |
| CRITICAL | | (54-65) | F100c, N101, | (24-27) | (50-56) | |
| | | | F102 | | | |
| POT. | M34, N35, | 151, T52a, | H97, G99, | H28, N29, | — | G91, Y92, |
| CRITICAL | V33 | R53 | N100a | N30, A31 | | S93, G94, |
| | | | | | | S95, T95a, |
| | | | | | | N95c, V97, |
| | | | | | | Y95b |
| CRITICAL | | Y50, W52 | I98, Y100, | W32 | — | H96 |
| | | | I100b | | | |

To analyze the effect of amino acid substitutions in the regions which are defined as potentially critical we used an energy minimization approach (details are provided below). A valid point mutation is accepted if it doesn't affect either hydrophobicity index, orientation of other amino acids, or excluded volume. As an initial guess for possible amino acid substitutions in the potential critical amino acids, the amino acid substitutions found in 7D4, 7E10, and 4H8 vis-à-vis 38F8 were considered. Results from the modelling of possible amino acid substitutions in the potentially critical regions are collected in Table 10 and are explained below. Possible mutations are divided into three groups: family variations (i.e. found in closely related 7D4 and 7E10), amino acid substitutions that are approved using in silico evaluation, and other suggested substitutions which have amino acids with similar physico-chemical characteristics to in silico approved substitutions.

TABLE 10
Possible point mutations in CDR regions of 38F8
for critical and potentially critical resiudes

| | | | Family | | |
|---|---|---|---|---|---|
| | | | Variations | In silico | Other |
| | Position | Residue | (7D4 or | approved | suggested |
| Chain | (Kabat) | (38F8) | 7E10) | exchanges | exchanges |
| Heavy | 33 | V | | A | |
| | 34 | M | L | I | V |
| | 35 | N | — | S, T | Q |
| | 51 | I | — | A, L | V |
| | 52a | T | — | S | — |
| | 53 | R | G, D | K | — |
| | 97 | H | A | R, K | — |
| | 99 | G | — | A | V |
| | 100a | N | A | Q | — |
| Light | 28 | H | Y | — | — |
| | 29 | N | K | — | — |
| | 30 | N | — | Q, S, T | — |
| | 31 | A | N | V | |
| | 91 | G | — | A | — |
| | 92 | Y | W | F | — |
| | 93 | S | — | T | — |
| | 94 | G | A, S | — | — |
| | 95 | S | G, N | — | Q |
| | 95a | T | S, G | — | — |
| | 95b | Y | | F | |
| | 95c | N | S | T | Q |
| | 97 | V | A | — | I, L |

### Summary of Amino Acid Substitution Analysis by CDRs

CDR-H1: Amino acids M34 and N35 are in the close proximity of V33 (which is involved in the binding mode). Mutations M34L(J) and N35S(T) do not affect conformation and orientation of V33 suggesting that these mutations do not affect the paratope binding and antibody characteristics. Mutations to amino acids with similar physico-chemical properties are possible (N35Q, M34V). V33 contributes to the interaction with L-T4 via a hydrophobic side chain interaction. Substitution V33A should according to the comparison of hydrophobicity indexes and excluded volumes not affect this interaction.

CDR-H2: Amino acids 151, T52a, R53 are in the close proximity of critical Y50 and W52. From the same clone family, mutation R53G is possible due to the small size of G, mutation to an amino acid from a different class is critical. Mutation R53D, due to its charge nature, is not critical, and the orientation is conserved. Mutations I51A(L), R53K and T52aS do not affect conformation and orientation of critical Y50 and W52. Y50 is involved in H-bonding, therefore exchanges at this position could be critical. W52 is a part of a hydrophobic interaction, proper orientation of aromatic parts is also critical for the binding point.

CDR-H3: Amino acids H97, G99, N100a are in the close proximity of critical 198, Y100, I100b. Mutations H97A and N100aA lead to a re-orientation of the critical IGY(98-100) fragment. As A is found in positions 97 and 100a of 7E10 and 7D4 it is evident that even if the orientation is changed, an antibody having A97 and/or A100a is still having an excellent binding affinity. kₐ- and Elecsys assay performance. Mutations H97R(K), N100aQ have been found to keep the proper orientation of the IGY(98-100) fragment and may thus be preferred substitutions. G99 is involved in binding through the backbone, as this allows it to mutate in a small amino acid, mutation G99A(V).

CDR-L1: Amino acid N30 is in close proximity to the paratope amino acids H28, N29, A31, W32. Mutation of N30Q(S, T) does not affect orientation of the fragment HNNAW(28-32). N30Q(S, T) are in the binding fragment HNNAW(28-32) but are facing the opposite direction and do not affect bonds with L-T4. Mutation H28K is critical; it leads to the wrong orientation of N29 and breaks of an aromatic bond in the binding motive. Mutation A31 slightly shifts position of W32 (and affect pi stacking), therefore this position should preferably stay small and hydrophobic, e.g. V. Position W32 is critical. Family variations are listed in Table 10.

CDR-L2: CDR-L2 can have various point mutations. CDR-L2 loop isn't involved directly into binding therefore it is classified as a non-critical region.

CDR-L3: Orientations of Y95b, H96 are critical for the binding mode. Y95b is involved in the hydrophobic interactions therefore it can be mutated in F. Mutations Y92W(F) and S93T are not critical, since these amino acids are involved through the backbone interaction, however the overall orientation is shifted. Mutations G91A, G94A(S), S95G(N), T95aS, N95cS(T), V97A do not affect conformation and orientation of critical Y95b, H96. Further mutations in specified above positions into amino acids with similar physical chemical properties are possible.

### Computer Simulation Details:

### 1) Molecular Dynamics Simulations.

To obtain the structures of 7E10, 7D4 and 4H8 Fabs, the GROMACS simulation package was used. Antibodies and environmental water were modeled in a fully atomistic representation in a canonical (NVT) ensemble (box size: ˜7.0×7.0×7.0 nm3) with a time step of 2 fs using AMBER99SB-ILDN [Lindorff-Larsen et al., Proteins 78, 1950-58, 2010] force field parameters, and the tip3p model [D. J. Price, and C. L. Brooks III, J. Chem. Phys. 121, 10096, 2004] for water. The temperature was set at 300 K by the velocity-rescale thermostat. Each dynamic trajectory was 300 ns long to sample of loop conformation.

### 2) Structure Optimization Upon Amino Acid Substitutions.

Amino acid substitutions in the crystal structure of 38F8 are conducted using the SAMSON model [OneAngstrom, SAMSON, 2020, Available from: https://www.samson-connect.net/], a computer software platform. After a certain point mutation in a crystal structure, the energy of the newly obtained structure was minimized to equilibrate the local degrees of freedom. This was done using FIRE (Fast Inertial Relaxation Engine) optimizer for molecular structures [Bitzek et al., Physical Review Letters, 97, 170201, 2006].Physical Review Letters, 97, 170201, 2006].

1. A monoclonal antibody specifically binding to L-thyroxine (T4), wherein said monoclonal antibody comprises:
i) a heavy chain variable domain (VH) comprising (a) a CDR-H1 having the amino acid sequence of X1NVX2N (wherein X1 is S or R and X2 is M or L), (b) a CDR-H2 having the amino acid sequence of SEQ ID NO: 20, and (c) a CDR-H3 having the amino acid sequence of SEQ ID NO: 21; and
ii) a light chain variable domain (VL) comprising (d) a CDR-L1 having the amino acid sequence of SEQ ID NO: 22 and (e) a CDR-L3 having the amino acid sequence of SEQ ID NO: 23.

2. The monoclonal antibody of claim 1, wherein the light chain variable domain comprises a CDR-L2 having the amino acid sequence of SEQ ID NO: 24.

3. The monoclonal antibody of claim 1, wherein at a temperature of 37° C. the association rate constant (kₐ) of the monoclonal antibody with T4 is 1.9*10⁷ M⁻¹sec⁻¹ or higher.

4. The monoclonal antibody of claim 1, wherein the association rate constant (kₐ) for the binding to T4 corresponds to at least 10% of the association rate constant (kₐ) for the binding to T4 of the antibody comprising or consisting of a heavy chain of SEQ ID NO: 48 and a light chain of SEQ ID NO: 49, wherein the association rate constants are measured under the identical experimental conditions.

5. The monoclonal antibody of claim 1, wherein the monoclonal antibody discriminates T4 from 3-iodo-L-Thyrosine (L-T3), rThyroid hormone (rT3), 3,3′,5-tri-iodo-thyroacetic acid, 3,3′,5,5′-tetra-iodothyroacetic acid, 3,5-di-iodo-L-Thyrosine and/or 3-i-L-Thyrosine.

6. The monoclonal antibody of claim 1 wherein the monoclonal antibody when used in a competitive immunoassay for quantifying T4 shows a signal-to-noise ratio that is at least 29%.

7. A polynucleotide encoding
(i) the heavy chain or heavy chain variable domain of the monoclonal antibody according to claim 1, and/or
(ii) the light chain or light chain variable domain of the monoclonal antibody according to claim 1.

8. A vector comprising the polynucleotide according to claim 7.

9. A host cell comprising the polynucleotide according to claim 7.

10. A method of producing the monoclonal antibody according to claim 1, said method comprising culturing a host cell and isolating said antibody.

11. A composition comprising the antibody according to claim 1.

12. An in vitro immunoassay method for quantifying T4 in a sample, said method comprising using the antibody as defined in claim 1.

13. A kit comprising the antibody as defined in claim 1.

14. The monoclonal antibody of claim 6, wherein the monoclonal antibody when used in a competitive immunoassay for quantifying T4 shows a signal-to-noise ratio that is at least 65%.

15. The monoclonal antibody of claim 6, wherein at least 95% of the signal-to-noise ratio achieved with a Fab fragment having a heavy chain sequence of SEQ ID NO: 48 and a light chain sequence of SEQ ID NO: 49 in the otherwise identical immunoassay setup.

16. The method of claim 12, wherein the T4 is free T4.

17. A monoclonal antibody specifically binding to L-thyroxine (T4), wherein said monoclonal antibody comprises:
i) a heavy chain variable domain (VH) comprising (a) a CDR-H1 having the amino acid sequence of SEQ ID NO: 12, (b) a CDR-H2 having the amino acid sequence of SEQ ID NO: 7, and (c) a CDR-H3 having the amino acid sequence of SEQ ID NO: 14; and
ii) a light chain variable domain (VL) comprising (d) a CDR-L1 having the amino acid sequence of SEQ ID NO: 15, (e) a CDR-L2 having the amino acid sequence of SEQ ID NO: 11, and (f) a CDR-L3 having the amino acid sequence of SEQ ID NO: 18.

18. A monoclonal antibody specifically binding to L-thyroxine (T4), wherein said monoclonal antibody comprises:
i) a heavy chain variable domain (VH) comprising (a) a CDR-H1 having the amino acid sequence of SEQ ID NO: 25, (b) a CDR-H2 having the amino acid sequence of SEQ ID NO: 27, and (c) a CDR-H3 having the amino acid sequence of SEQ ID NO: 29; and
a light chain variable domain (VL) comprising (d) a CDR-L1 having the amino acid sequence of SEQ ID NO: 30, (e) a CDR-L2 having the amino acid sequence of SEQ ID NO: 33, and (f) a CDR-L3 having the amino acid sequence of SEQ ID NO: 31; or
ii) a heavy chain variable domain (VH) comprising (a) a CDR-H1 having the amino acid sequence of SEQ ID NO: 26, (b) a CDR-H2 having the amino acid sequence of SEQ ID NO: 28, and (c) a CDR-H3 having the amino acid sequence of SEQ ID NO: 29; and
a light chain variable domain (VL) comprising (d) a CDR-L1 having the amino acid sequence of SEQ ID NO: 30, (e) a CDR-L2 having the amino acid sequence of SEQ ID NO: 33, and (f) a CDR-L3 having the amino acid sequence of SEQ ID NO: 32.

19. (canceled)


SEQUENCE LISTING (61 sequences)

SEQ ID NO: 1 - protein, 12 aa, synthetic construct
  REGION 1..12: note = Amino acids (AS) 54 to 65 of the VH of 38F8 according to Kabatnumbering
SGNTYYATWAKG

SEQ ID NO: 2 - protein, 12 aa, synthetic construct
  REGION 1..12: note = AS 54 to 65 of the VH of 7D4 according to Kabat numbering
SGSTYYATWAKG

SEQ ID NO: 3 - protein, 12 aa, synthetic construct
  REGION 1..12: note = AS 54 to 65 of the VH of 7E10 according to Kabat numbering
SGSTYYATWAKG

SEQ ID NO: 4 - protein, 7 aa, synthetic construct
  REGION 1..7: note = AS 50 to 56 of the VL of 38F8 according to Kabat numbering
GASTLTS

SEQ ID NO: 5 - protein, 7 aa, synthetic construct
  REGION 1..7: note = AS 50 to 56 of the VL of 7E10 and 7D4 according to Kabatnumbering
GASTLAS

SEQ ID NO: 6 - protein, 5 aa, synthetic construct
  REGION 1..5: note = CDR-H1 sequence of 38F8 with variations in the paratope residues
  VARIANT 3: note = X is V or A
SNXMN

SEQ ID NO: 7 - protein, 17 aa, synthetic construct
  REGION 1..17: note = CDR-H2 sequence of 38F8
YIWTRSGNTYYASWAKG

SEQ ID NO: 8 - protein, 11 aa, synthetic construct
  REGION 1..11: note = CDR-H3 sequence of 38F8 with variations in the paratope residues
  VARIANT 5: note = X is G, A or V
GLHIXYNIFNF

SEQ ID NO: 9 - protein, 13 aa, synthetic construct
  REGION 1..13: note = CDR-L1 sequence of 38F8 with variations in the paratope residues
  VARIANT 7: note = X is H or Y
  VARIANT 8: note = X is N or K
QSSQSVXXNAWCS

SEQ ID NO: 10 - protein, 12 aa, synthetic construct
  REGION 1..12: note = CDR-L3 sequence of 38F8 with variations in the paratope residues
  VARIANT 3: note = X is G or A
  VARIANT 4: note = X is Y, W or F
  VARIANT 5: note = X is S or T
  VARIANT 9: note = X is Y or F
  VARIANT 10: note = X is N, S, T or Q
AGXXXGSTXXHV

SEQ ID NO: 11 - protein, 7 aa, synthetic construct
  REGION 1..7: note = CDR-L2 sequence of 38F8
GASTLTS

SEQ ID NO: 12 - protein, 5 aa, synthetic construct
  REGION 1..5: note = CDR-H1 sequence of 38F8
SNVMN

SEQ ID NO: 13 - protein, 11 aa, synthetic construct
  REGION 1..11: note = CDR-H3 sequence of 38F8 with variations in the paratope residues
  VARIANT 5: note = X is G or A
GLHIXYNIFNF

SEQ ID NO: 14 - protein, 11 aa, synthetic construct
  REGION 1..11: note = CDR-H3 sequence of 38F8
GLHIGYNIFNF

SEQ ID NO: 15 - protein, 13 aa, synthetic construct
  REGION 1..13: note = CDR-L1 sequence of 38F8
QSSQSVHNNAWCS

SEQ ID NO: 16 - protein, 12 aa, synthetic construct
  REGION 1..12: note = CDR-L3 sequence of 38F8 with variations in the paratope residues
  VARIANT 3: note = X is G or A
  VARIANT 4: note = X is Y, W or F
  VARIANT 5: note = X is S or T
  VARIANT 9: note = X is Y or F
  VARIANT 10: note = X is N, S or T
AGXXXGSTXXHV

SEQ ID NO: 17 - protein, 12 aa, synthetic construct
  REGION 1..12: note = CDR-L3 sequence of 38F8 with variations in the paratope residues
  VARIANT 4: note = X is Y or W
  VARIANT 10: note = X is N or S
AGGXSGSTYXHV

SEQ ID NO: 18 - protein, 12 aa, synthetic construct
  REGION 1..12: note = CDR-L3 sequence of 38F8
AGGYSGSTYNHV

SEQ ID NO: 19 - skipped

SEQ ID NO: 20 - protein, 17 aa, synthetic construct
  REGION 1..17: note = CDR-H2 consensus sequence of 38F8, 7E10 and 7D4
  VARIANT 5: note = X is R, G or D
  VARIANT 8: note = X is N or S
  VARIANT 13: note = X is S or T
YIWTXSGXTYYAXWAKG

SEQ ID NO: 21 - protein, 11 aa, synthetic construct
  REGION 1..11: note = CDR-H3 consensus sequence of 38F8, 7E10 and 7D4
  VARIANT 3: note = X is H or A
  VARIANT 7: note = X is N or A
GLXIGYXIFNF

SEQ ID NO: 22 - protein, 13 aa, synthetic construct
  REGION 1..13: note = CDR-L1 consensus sequence of 38F8, 7E10 and 7D4
  VARIANT 7: note = X is H or Y
  VARIANT 8: note = X is N or K
  VARIANT 10: note = X is A or N
  VARIANT 12: note = X is C or L
QSSQSVXXNXWXS

SEQ ID NO: 23 - protein, 12 aa, synthetic construct
  REGION 1..12: note = CDR-L3 consensus sequence of 38F8, 7E10 and 7D4
  VARIANT 4: note = X is Y or W
  VARIANT 6: note = X is G, A or S
  VARIANT 7: note = X is S, G or N
  VARIANT 8: note = X is T, G or S
  VARIANT 10: note = X is N or S
  VARIANT 12: note = X is V or A
AGGXSXXXYXHX

SEQ ID NO: 24 - protein, 7 aa, synthetic construct
  REGION 1..7: note = CDR-L2 consensus sequence of 38F8, 7E10 and 7D4
  VARIANT 6: note = X is T or A
GASTLXS

SEQ ID NO: 25 - protein, 5 aa, synthetic construct
  REGION 1..5: note = CDR-H1 of 7E10
RNVMN

SEQ ID NO: 26 - protein, 5 aa, synthetic construct
  REGION 1..5: note = CDR-H1 of 7D4
RNVLN

SEQ ID NO: 27 - protein, 17 aa, synthetic construct
  REGION 1..17: note = CDR-H2 of 7E10
YIWTDSGSTYYATWAKG

SEQ ID NO: 28 - protein, 17 aa, synthetic construct
  REGION 1..17: note = CDR-H2 of 7D4
YIWTGSGNTYYATWAKQ

SEQ ID NO: 29 - protein, 11 aa, synthetic construct
  REGION 1..11: note = CDR-H3 of 7D4 and 7E10
GLAIGYAIFNF

SEQ ID NO: 30 - protein, 13 aa, synthetic construct
  REGION 1..13: note = CDR-L1 of 7D4 and 7E10
QSSQSVYKNNWLS

SEQ ID NO: 31 - protein, 12 aa, synthetic construct
  REGION 1..12: note = CDR-L3 of 7E10
AGGWSSNSYNHA

SEQ ID NO: 32 - protein, 12 aa, synthetic construct
  REGION 1..12: note = CDR-L3 of 7D4
AGGWSAGGYSHA

SEQ ID NO: 33 - protein, 7 aa, synthetic construct
  REGION 1..7: note = CDR-L2 of 7D4 and 7E10
GASTLAS

SEQ ID NO: 34 - protein, 29 aa, synthetic construct
  REGION 1..29: note = FW-H1 of 38F8 according to Kabat
LSLEESGGRLVTPGTPLTLTCTVSGIDLS

SEQ ID NO: 35 - protein, 14 aa, synthetic construct
  REGION 1..14: note = FW-H2 of 38F8 according to Kabat
WVRQAPGKGLEWIG

SEQ ID NO: 36 - protein, 31 aa, synthetic construct
  REGION 1..31: note = FW-H3 of 38F8 according to Kabat
RFTISKTSSTTVDLKMTSLTTEDTATYFCAG

SEQ ID NO: 37 - protein, 11 aa, synthetic construct
  REGION 1..11: note = FW-H4 of 38F8 according to Kabat
WGQGTLVTVSS

SEQ ID NO: 38 - protein, 22 aa, synthetic construct
  REGION 1..22: note = FW-L1 of 38F8 according to Kabat
AVLTQTPSPVSAAVGGTVTINC

SEQ ID NO: 39 - protein, 15 aa, synthetic construct
  REGION 1..15: note = FW-L2 of 38F8 according to Kabat
WFQKKPGQPPKQLIY

SEQ ID NO: 40 - protein, 32 aa, synthetic construct
  REGION 1..32: note = FW-L3 of 38F8 according to Kabat
GVPSRFKGSGSGTQFTLTISDVQCDDAATYYC

SEQ ID NO: 41 - protein, 10 aa, synthetic construct
  REGION 1..10: note = FW-L4 of 38F8 according to Kabat
FGGGTEVVVK

SEQ ID NO: 42 - protein, 118 aa, synthetic construct
  REGION 1..118: note = VH domain of 38F8
LSLEESGGRLVTPGTPLTLTCTVSGIDLSSNVMNWVRQAPGKGLEWIGYIWTRSGNTYYASWAKGRFTISKTSSTTVDLKMTSLTTEDTATYFCAGGLHIGYNIFNFWGQGTLVTVSS

SEQ ID NO: 43 - protein, 118 aa, synthetic construct
  REGION 1..118: note = VH domain of 7E10
QSVEESGGRLVTPGTPLTLTCTVSGIDLSRNVMNWVRQAPGKGLEWIGYIWTDSGSTYYATWAKGRFTISKTSSTTVELKMTSPTTEDTATYFCAGGLAIGYAIFNFWGQGTLVTVSS

SEQ ID NO: 44 - protein, 118 aa, synthetic construct
  REGION 1..118: note = VH domain of 7D4
QSVEESGGRLVTPGTPLTLTCTVSGIDLSRNVLNWVRQAPGKGLEWIGYIWTGSGNTYYATWAKGRFTISKTSSTTVDLKMTSPTTEDTATYFCAGGLAIGYAIFNFWGQGTLVTVSS

SEQ ID NO: 45 - protein, 111 aa, synthetic construct
  REGION 1..111: note = VL domain of 38F8
AVLTQTPSPVSAAVGGTVTINCQSSQSVHNNAWCSWFQKKPGQPPKQLIYGASTLTSGVPSRFKGSGSGTQFTLTISDVQCDDAATYYCAGGYSGSTYNHVFGGGTEVVVK

SEQ ID NO: 46 - protein, 111 aa, synthetic construct
  REGION 1..111: note = VL domain of 7E10
AVLTQTPSPVSAAVGGTVTISCQSSQSVYKNNWLSWFQQKPGQPPKLLIYGASTLASGVPSRFEGSGSGTQFTLTISDVQCDDAATYYCAGGWSSNSYNHAFGGGTGVVVT

SEQ ID NO: 47 - protein, 112 aa, synthetic construct
  REGION 1..112: note = VL domain of 7D4
AAVLTQTPSPVSAAVGGTVTISCQSSQSVYKNNWLSWFQQKPGQPPKLLIYGASTLASGVPSRFEGSGSGTQFTLTISDVQCDDAATYYCAGGWSAGGYSHAFGGGTGVVVA

SEQ ID NO: 48 - protein, 219 aa, synthetic construct
  REGION 1..219: note = Heavy chain of Fab 38F8
LSLEESGGRLVTPGTPLTLTCTVSGIDLSSNVMNWVRQAPGKGLEWIGYIWTRSGNTYYASWAKGRFTISKTSSTTVDLKMTSLTTEDTATYFCAGGLHIGYNIFNFWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCS

SEQ ID NO: 49 - protein, 215 aa, synthetic construct
  REGION 1..215: note = Light chain of Fab 38F8
AVLTQTPSPVSAAVGGTVTINCQSSQSVHNNAWCSWFQKKPGQPPKQLIYGASTLTSGVPSRFKGSGSGTQFTLTISDVQCDDAATYYCAGGYSGSTYNHVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 50 - protein, 126 aa, synthetic construct
  REGION 1..126: note = VH domain of 18B3
QSVEESGGRLVTPGTPLTLTCTLSGFSLKGYALSWVRQAPGKGLEWIGLIGNTGMTYYATWATGRFTISKTSTTVDLKMTSPTTEDTATYFCARDWFRYDTFGGTTVIYYYGMDLWGPGTLVTVSS

SEQ ID NO: 51 - protein, 114 aa, synthetic construct
  REGION 1..114: note = VH domain of 4H8
QSVEESGGRLVTPGTPLTLTCTASGFSLSAYYMIWVRQAPGKGLEWIGYIGGGVSASYASWANGRFTISSTSTTVDLKIPSPTTEDTATYFCARGSWNSGIDLWGQGTLVTVSS

SEQ ID NO: 52 - protein, 122 aa, synthetic construct
  REGION 1..122: note = VH domain of 3B7
QSLEESGGDLVKPGASLTLTCKASGIDFSGSALCWVRQAPGKGPEWIVCIYVGSFQNTYYASWAKGRFTISKTSSTTVTLQMTSLTVADTATYFCASDASGISHYRYYFNLWGPGTLVTVSS

SEQ ID NO: 53 - protein, 115 aa, synthetic construct
  REGION 1..115: note = VL domain of 18B3
ALVMTQTPSPVSAAVGGTVTINCQASEEIGNNLAWFQQKPGQPPKLLIQRASTLASGVPSRFSGSGSGTDYSLTISGLQCDDAATYYCLGVLPYIGADGHAFGGGTEVVVKGDPV

SEQ ID NO: 54 - protein, 115 aa, synthetic construct
  REGION 1..115: note = VL domain of 4H8
AAVLTQTASPVSAAVGGTVTINCQSSQSVVNNNRLSWFQQKPGQPPKLLIYKASTLASGVPSRFKGSGSGTQFTLTISDVQCDDAATYYCLGGYISTSDNAFGGGTEVVVKGDPV

SEQ ID NO: 55 - protein, 116 aa, synthetic construct
  REGION 1..116: note = VL domain of 3B7
AIEMTQTPFSVSAAVGGTVTISCQASESVYAKLGWYQQKPGQPPKLLIYDASSLASGVPSRFKGSGSGTEYSLTISDLECDDAATYYCQSAYYTRGADTWGAFGGGTEVVVKGDPV

SEQ ID NO: 56 - protein, 5 aa, synthetic construct
  REGION 1..5: note = CDR-H1 of 4H8
AYYMI

SEQ ID NO: 57 - protein, 16 aa, synthetic construct
  REGION 1..16: note = CDR-H2 of 4H8
YIGGGVSASYASWANG

SEQ ID NO: 58 - protein, 9 aa, synthetic construct
  REGION 1..9: note = CDR-H3 of 4H8
GSWNSGIDL

SEQ ID NO: 59 - protein, 13 aa, synthetic construct
  REGION 1..13: note = CDR-L1 of 4H8
QSSQSVVNNNRLS

SEQ ID NO: 60 - protein, 7 aa, synthetic construct
  REGION 1..7: note = CDR-L2 of 4H8
KASTLAS

SEQ ID NO: 61 - protein, 11 aa, synthetic construct
  REGION 1..11: note = CDR-L3 of 4H8
LGGYISTSDNA